SEQUENCE LISTING (62 sequences)

SEQ ID NO: 1 - DNA, 6 bp, Artificial Sequence
  Description of Artificial Sequence Synthetic oligonucleotide
ggtacc

SEQ ID NO: 2 - DNA, 18 bp, Artificial Sequence
  Description of Artificial Sequence Synthetic oligonucleotide
ggatccactagtcaattg

SEQ ID NO: 3 - protein, 8 aa, Artificial Sequence
  Description of Artificial Sequence Synthetic peptide
SIINFEKL

SEQ ID NO: 4 - DNA, 6 bp, Artificial Sequence
  Description of Artificial Sequence Synthetic oligonucleotide
cggccg

SEQ ID NO: 5 - DNA, 639 bp, Artificial Sequence
  Description of Artificial Sequence Synthetic polynucleotide
ggtaccgggaagcagttggggttaactgattaacaaatgttagagaaaaattaattctccaagtgatattcttaaaataattcatgaatattttttcttatattagctaattaagaagataattaactgctaatccaatttttaacggaataaattagtgaaaatgaaggccgaattttccttgttctaaaaaggttgtattagcgtatcacgaggagggagtataagtgggattaaatagatttatgcgtgcgatgatggtagttttcattactgccaactgcattacgattaaccccgacataatatttgcagcgacagatagcgaagattccagtctaaacacagatgaatgggaagaagaaaaaacagaagagcagccaagcgaggtaaatacgggaccaagatacgaaactgcacgtgaagtaagttcacgtgatattgaggaactagaaaaatcgaataaagtgaaaaatacgaacaaagcagacctaatagcaatgttgaaagcaaaagcagagaaaggtggatccactagtcaattgggtgacggtagtattaaacttagcaaagtattacaattagaaagtattattaattttgaaaaattagctgatggttcagttaaataagcggccg

SEQ ID NO: 6 - DNA, 717 bp, Artificial Sequence
  Description of Artificial Sequence Synthetic polynucleotide
ggatcccaagaatatcagtgttatggaagtagtagcaatactcgcgttttgaatgaactaaattatgataacgcaggtacaaacttatacaatgaattagaaatgaattattacggtaaacaagaaaattggtattcgctaaagaagaatagtcgctcattaggcgagaacgatgatggtaataacgaagataatgagaaattacgaaaacctaaacataagaaacttaaacagccggcagatggaaatccagacccaaatgcaaatccaaatgttgatccaaatgcgaatccgaatgtaaatgctaacccgaacgctaatcctaacgcaaatcctaataaaaataatcaaggaaatggccaaggacataatatgccaaatgatcctaatcgtaatgtcgatgaaaatgctaacgctaattcggcagttaaaaacaataataacgaggaaccaagtgacaaacatattaaagaatatctaaacaaaattcaaaatagtttatcaacggaatggtcgccatgcagtgttacgtgtggcaatggcatacaagtgcgcattaaacctggttcagcgaataaaccgaaagacgaattagattatgcaaatgatattgagaaaaagatttgtaaaatggaaaaatgtagttcagtcttcaatgtagtgaatagctcaataggcttaattatggttcttagcttcctttttctaaacactagt

SEQ ID NO: 7 - protein, 235 aa, Artificial Sequence
  Description of Artificial Sequence Synthetic polypeptide
QEYQCYGSSSNTRVLNELNYDNAGTNLYNELEMNYYGKQENWYSLKKNSRSLGENDDGNNEDNEKLRKPKHKKLKQPADGNPDPNANPNVDPNANPNVNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVFNVVNSSIGLIMVLSFLFLN

SEQ ID NO: 8 - DNA, 684 bp, Artificial Sequence
  Description of Artificial Sequence Synthetic polynucleotide
ggatcccaagaatatcagtgttatggaagtagtagcaatactcgcgttttgaatgaactaaattatgataacgcaggtacaaacttatacaatgaattagaaatgaattattacggtaaacaagaaaattggtattcgctaaagaagaatagtcgctcattaggcgagaacgatgatggtaataacgaagataatgagaaattacgaaaacctaaacataagaaacttaaacagccggcagatggaaatccagacccaaatgcaaatccaaatgttgatccaaatgcgaatccgaatgtaaatgctaacccgaacgctaatcctaacgcaaatcctaataaaaataatcaaggaaatggccaaggacataatatgccaaatgatcctaatcgtaatgtcgatgaaaatgctaacgctaattcggcagttaaaaacaataataacgaggaaccaagtgacaaacatattaaagaatatctaaacaaaattcaaaatagtttatcaacggaatggtcgccatgcagtgttacgtgtggcaatggcatacaagtgcgcattaaacctggttcagcgaataaaccgaaagacgaattagattatgcaaatgatattgagaaaaagatttgtaaaatggaaaaatgtagttcagtcttcaatgtagtgaatagctcaataggcactagt

SEQ ID NO: 9 - protein, 224 aa, Artificial Sequence
  Description of Artificial Sequence Synthetic polypeptide
QEYQCYGSSSNTRVLNELNYDNAGTNLYNELEMNYYGKQENWYSLKKNSRSLGENDDGNNEDNEKLRKPKHKKLKQPADGNPDPNANPNVDPNANPNVNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVFNVVNSSIG

SEQ ID NO: 10 - DNA, 486 bp, Artificial Sequence
  Description of Artificial Sequence Synthetic polynucleotide
ggatccttccgaggtaataacggacataattcatcgtcttccttatataacgggagccaatttatagaacaacttaataacagttttacaagtgcatttttggagtcacagagtatgaataaaatcggtgatgatctagcagaaacaatctcaaacgaattagtcagtgttcttcaaaaaaactcaccaacatttcttgaatcgtccttcgacatcaaaagtgaagtaaagaaacatgcgaaaagtatgcttaaagagcttattaaagtgggcttgccatcgtttgaaaacctagtagcggagaatgtaaaacctcctaaggtcgatccggcgacctatggtatcatcgtgccagttttaacatctttgtttaacaaagtagaaactgctgtaggagctaaagtatcggatgaaatttggaactataattcgccggatgttagcgagtctgaagaatcgctaagtgatgatttcttcgacactagt

SEQ ID NO: 11 - protein, 158 aa, Artificial Sequence
  Description of Artificial Sequence Synthetic polypeptide
FRGNNGHNSSSSLYNGSQFIEQLNNSFTSAFLESQSMNKIGDDLAETISNELVSVLQKNSPTFLESSFDIKSEVKKHAKSMLKELIKVGLPSFENLVAENVKPPKVDPATYGIIVPVLTSLFNKVETAVGAKVSDEIWNYNSPDVSESEESLSDDFFD

SEQ ID NO: 12 - DNA, 1446 bp, Artificial Sequence
  Description of Artificial Sequence Synthetic polynucleotide
ggatccatgggtacaaacagtgaaaaagatgagataatcaaaagcaatttacgatctggttcgtctaacagtcgtaaccgtatcaatgaagaaaaacatgaaaagaaacacgtattatcgcataatagctatgagaaaaccaaaaacaatgagaataataaattttttgataaagacaaggagttaacaatgtccaatgtaaagaacgtatcccaaacgaatttcaaatcacttttacgtaacttaggtgtgtccgaaaatatcttcttaaaagagaacaaattgaataaagagggtaaactaattgaacacattattaatgatgatgacgacaaaaagaaatatatcaaaggccaagacgagaatcgtcaagaagatcttgaagaaaaggcggcagaacaacaaagtgatcttgaacaggaaagacttgctaaagagaaattgcaagaacaacagtctgatttagagcaagagcgtttagcgaaagaaaaattacaagaacgactagcaaaagaaaaactacaagagcaacaacgcgatttggaacaggaacgtttggcaaaagagaaacttcaagaacagcaacgcgatcttgaacaacgaaaagcagataccaagaagaatttagaacgcaagaaagaacacggggacgttcttgccgaagatttatatgggcgattagaaatcccagccatcgaattaccatctgaaaatgaacgaggctattatatcccacatcaatcaagccttcctcaggataacagaggtaatagcagagattctaaagaaatttcaattatagagaaaacgaatagagaaagtatcactacaaacgtagaaggacgccgtgatattcataaaggacatttggaagagaagaaagatgggtctatcaaaccggaacagaaggaagataaatccgctgacattcaaaatcacactcttgaaacagttaacattagcgacgtgaacgattttcaaatttctaaatatgaagatgaaattagcgctgaatatgatgattcgcttattgacgaagaagaagatgatgaagaccttgatgaatttaaaccgattgttcaatatgataattttcaagatgaagagaatattggaatctataaggaattagaagatttaatcgagaaaaatgaaaatttagatgatcttgacgaaggtattgaaaaatcctctgaagaactttccgaagagaaaattaagaaaggtaaaaagtacgagaaaactaaagacaacaatttcaaaccaaatgataaaagcctttatgacgagcatattaaaaagtataaaaacgataaacaagtcaataaagaaaaagagaagtttatcaaatctctatttcacatttttgacggtgacaatgaaatccttcaaattgtagatgaattgtccgaagatatcacaaagtattttatgaaattaactagt

SEQ ID NO: 13 - protein, 475 aa, Artificial Sequence
  Description of Artificial Sequence Synthetic polypeptide
NSEKDEIIKSNLRSGSSNSRNRINEEKHEKKHVLSHNSYEKTKNNENNKFFDKDKELTMSNVKNVSQTNFKSLLRNLGVSENIFLKENKLNKEGKLIEHIINDDDDKKKYIKGQDENRQEDLEEKAAEQQSDLEQERLAKEKLQEQQSDLEQERLAKEKLQERLAKEKLQEQQRDLEQERLAKEKLQEQQRDLEQRKADTKKNLERKKEHGDVLAEDLYGRLEIPAIELPSENERGYYIPHQSSLPQDNRGNSRDSKEISIIEKTNRESITTNVEGRRDIHKGHLEEKKDGSIKPEQKEDKSADIQNHTLETVNISDVNDFQISKYEDEISAEYDDSLIDEEEDDEDLDEFKPIVQYDNFQDEENIGIYKELEDLIEKNENLDDLDEGIEKSSEELSEEKIKKGKKYEKTKDNNFKPNDKSLYDEHIKKYKNDKQVNKEKEKFIKSLFHIFDGDNEILQIVDELSEDITKYFMKL

SEQ ID NO: 14 - DNA, 1620 bp, Artificial Sequence
  Description of Artificial Sequence Synthetic polynucleotide
ggatccaatggtagagatgtacagaacaatatcgtagatgagatcaaataccgcgaagaagtttgcaatgatgaagttgatctttacttgttaatggattgttcaggttcaattcgtcgtcataactgggtcaatcacgcggttcctttggctatgaaacttattcaacaactaaacctaaatgaatctgcgattcacttgtatgttaacatattctcgaacaatgcgaaagaaatcattcgtttacattcggatgcaagcaagaataaagaaaaagcgttgataatcatacgaagcttactaagcactaatcttccgtatggccgaacaaacttatctgatgcattacttcaggttagaaaacatttgaatgatcgcattaaccgtgaaaatgcaaatcagttggttgtgattctaactgatgggattcctgatagcattcaagatagtcttaaagaatcacgaaaactaaatgaccgtggtgtgaaaatcgcagtttttggaattggacaaggcatcaatgttgctttcaatcgattcttagtcgggtgtcatccatccgacggaaagtgcaatttgtatgctgattctgcgtgggagaatgtgaaaaacgttattggaccattcatgaaagccgtatgtgttgaagtagaaaagacagctagttgcggtgtgtgggacgaatggtcaccatgtagtgtgacatgtggcaaaggcacacgctctcgcaaacgtgaaatacttcacgaaggatgcaccagtgaattacaagaacaatgtgaagaagaacgttgtccgccaaaacgtgaaccactagatgtacctgatgaaccagaagatgaccaaccgcgtccgcgtggtgacaactttgctgttgagaaacctgaagagaatatcattgacaataacccacaagagccatccccaaacccagaggaaggtaaaggggaaaatccaaatggtttcgacttagatgaaaatccagaaaatccaccaaatccggatattccacaacaagaaccaaacattccagaagattctgaaaaagaagtacctagtgatgtaccaaagaatccggaggacgatagagaagaaaactttgatattcctaagaaaccggaaaacaaacacgataatcaaaacaatcttccaaacgacaaatcagatagatccattccttatagtcctttaccaccaaaagtacttgataatgaacgcaaacaatcggacccacaatctcaagacaacaatgggaatcgtcatgtgccaaatagcgaagatagagaaactagacctcatggtcgtaacaatgagaatcgatcatacaatcgcaaatacaatgatacgccaaaacatccagaaagagaagaacatgaaaaaccggataacaataagaaaaagggaggtagtgacaacaagtataagattgcaggtggcattgcaggcggattagcattacttgcttgcgcaggcttagcctacaaattcgtagtcccgggtgcagctacgccttatgccggagagccagctccgtttgatgaaacattaggagaagaagataaggatttagatgagcctgagcaattcagattacctgaagaaaatgaatggaatcaattg

SEQ ID NO: 15 - protein, 536 aa, Artificial Sequence
  Description of Artificial Sequence Synthetic polypeptide
NGRDVQNNIVDEIKYREEVCNDEVDLYLLMDCSGSIRRHNWVNHAVPLAMKLIQQLNLNESAIHLYVNIFSNNAKEIIRLHSDASKNKEKALIIIRSLLSTNLPYGRTNLSDALLQVRKHLNDRINRENANQLVVILTDGIPDSIQDSLKESRKLNDRGVKIAVFGIGQGINVAFNRFLVGCHPSDGKCNLYADSAWENVKNVIGPFMKAVCVEVEKTASCGVWDEWSPCSVTCGKGTRSRKREILHEGCTSELQEQCEEERCPPKREPLDVPDEPEDDQPRPRGDNFAVEKPEENIIDNNPQEPSPNPEEGKGENPNGFDLDENPENPPNPDIPQQEPNIPEDSEKEVPSDVPKNPEDDREENFDIPKKPENKHDNQNNLPNDKSDRSIPYSPLPPKVLDNERKQSDPQSQDNNGNRHVPNSEDRETRPHGRNNENRSYNRKYNDTPKHPEREEHEKPDNNKKKGGSDNKYKIAGGIAGGLALLACAGLAYKFVVPGAATPYAGEPAPFDETLGEEDKDLDEPEQFRLPEENEWN

SEQ ID NO: 16 - DNA, 1434 bp, Artificial Sequence
  Description of Artificial Sequence Synthetic polynucleotide
ggatccaatggtagagatgtacagaacaatatcgtagatgagatcaaataccgcgaagaagtttgcaatgatgaagttgatctttacttgttaatggattgttcaggttcaattcgtcgtcataactgggtcaatcacgcggttcctttggctatgaaacttattcaacaactaaacctaaatgaatctgcgattcacttgtatgttaacatattctcgaacaatgcgaaagaaatcattcgtttacattcggatgcaagcaagaataaagaaaaagcgttgataatcatacgaagcttactaagcactaatcttccgtatggccgaacaaacttatctgatgcattacttcaggttagaaaacatttgaatgatcgcattaaccgtgaaaatgcaaatcagttggttgtgattctaactgatgggattcctgatagcattcaagatagtcttaaagaatcacgaaaactaaatgaccgtggtgtgaaaatcgcagtttttggaattggacaaggcatcaatgttgctttcaatcgattcttagtcgggtgtcatccatccgacggaaagtgcaatttgtatgctgattctgcgtgggagaatgtgaaaaacgttattggaccattcatgaaagccgtatgtgttgaagtagaaaagacagctagttgcggtgtgtgggacgaatggtcaccatgtagtgtgacatgtggcaaaggcacacgctctcgcaaacgtgaaatacttcacgaaggatgcaccagtgaattacaagaacaatgtgaagaagaacgttgtccgccaaaacgtgaaccactagatgtacctgatgaaccagaagatgaccaaccgcgtccgcgtggtgacaactttgctgttgagaaacctgaagagaatatcattgacaataacccacaagagccatccccaaacccagaggaaggtaaaggggaaaatccaaatggtttcgacttagatgaaaatccagaaaatccaccaaatccggatattccacaacaagaaccaaacattccagaagattctgaaaaagaagtacctagtgatgtaccaaagaatccggaggacgatagagaagaaaactttgatattcctaagaaaccggaaaacaaacacgataatcaaaacaatcttccaaacgacaaatcagatagatccattccttatagtcctttaccaccaaaagtacttgataatgaacgcaaacaatcggacccacaatctcaagacaacaatgggaatcgtcatgtgccaaatagcgaagatagagaaactagacctcatggtcgtaacaatgagaatcgatcatacaatcgcaaatacaatgatacgccaaaacatccagaaagagaagaacatgaaaaaccggataacaataagaaaaagggaggtagtgacaacaagtataagattcaattg

SEQ ID NO: 17 - protein, 474 aa, Artificial Sequence
  Description of Artificial Sequence Synthetic polypeptide
NGRDVQNNIVDEIKYREEVCNDEVDLYLLMDCSGSIRRHNWVNHAVPLAMKLIQQLNLNESAIHLYVNIFSNNAKEIIRLHSDASKNKEKALIIIRSLLSTNLPYGRTNLSDALLQVRKHLNDRINRENANQLVVILTDGIPDSIQDSLKESRKLNDRGVKIAVFGIGQGINVAFNRFLVGCHPSDGKCNLYADSAWENVKNVIGPFMKAVCVEVEKTASCGVWDEWSPCSVTCGKGTRSRKREILHEGCTSELQEQCEEERCPPKREPLDVPDEPEDDQPRPRGDNFAVEKPEENIIDNNPQEPSPNPEEGKGENPNGFDLDENPENPPNPDIPQQEPNIPEDSEKEVPSDVPKNPEDDREENFDIPKKPENKHDNQNNLPNDKSDRSIPYSPLPPKVLDNERKQSDPQSQDNNGNRHVPNSEDRETRPHGRNNENRSYNRKYNDTPKHPEREEHEKPDNNKKKGGSDNKYKI

SEQ ID NO: 18 - protein, 182 aa, Plasmodium falciparum
MNALRRLPVICSFLVFLVFSNVLCFRGNNGHNSSSSLYNGSQFIEQLNNSFTSAFLESQSMNKIGDDLAETISNELVSVLQKNSPTFLESSFDIKSEVKKHAKSMLKELIKVGLPSFENLVAENVKPPKVDPATYGIIVPVLTSLFNKVETAVGAKVSDEIWNYNSPDVSESEESLSDDFFD

SEQ ID NO: 19 - protein, 158 aa, Artificial Sequence; identical to SEQ ID NO: 11
  Description of Artificial Sequence Synthetic polypeptide

SEQ ID NO: 20 - protein, 397 aa, Plasmodium falciparum
MMRKLAILSVSSFLFVEALFQEYQCYGSSSNTRVLNELNYDNAGTNLYNELEMNYYGKQENWYSLKKNSRSLGENDDGNNEDNEKLRKPKHKKLKQPADGNPDPNANPNVDPNANPNVDPNANPNVDPNANPNANPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVFNVVNSSIGLIMVLSFLFLN

SEQ ID NO: 21 - protein, 235 aa, Artificial Sequence; identical to SEQ ID NO: 7
  Description of Artificial Sequence Synthetic polypeptide

SEQ ID NO: 22 - protein, 1909 aa, Plasmodium falciparum
MKHILYISFYFILVNLLIFHINGKIIKNSEKDEIIKSNLRSGSSNSRNRINEEKHEKKHVLSHNSYEKTKNNENNKFFDKDKELTMSNVKNVSQTNFKSLLRNLGVSENIFLKENKLNKEGKLIEHIINDDDDKKKYIKGQDENRQEDLEEKAAKETLQGQQSDLEQERLAKEKLQEQQSDSEQERLAKEKLQEQQSDLEQERLAKEKLQEQQSDLEQERLAKEKLQEQQSDLEQERRAKEKLQEQQSDLEQERRAKEKLQEQQSDLEQERRAKEKLQEQQSDLEQERLAKEKLQEQQSDLEQERRAKEKLQEQQSDLEQERLAKEKLQEQQSDLEQERLAKEKLQEQQSDLEQERLAKEKLQGQQSDLEQERLAKEKLQEQQSDLEQDRLAKEKLQEQQSDLEQERLAKEKLQEQQSDLEQERRAKEKLQEQQSDLEQERLAKEKLQEQQSDLEQERRAKEKLQEQQSDLEQERRAKEKLQEQQSDLEQERLAKEKLQEQQSDLEQERLAKEKLQEQQSDSEQERLAKEKLQEQQSDLEQERLAKEKLQEQQSDLEQERLAKEKLQEQQSDLEQERLAKEKLQEQQSDLEQERLAKEKLQGQQSDLEQERLAKEKLQGQQSDLEQERLAKEKLQEQQSDLEQERLAKEKLQEQQSDLERTKASKETLQEQQSDLEQERLAKEKLQEQQSDLEQERRAKEKLQEQQSDLEQERRAKEKLQEQQSDLEQERRAKEKLQEQQSDLEQERRAKEKLQEQQSDLEQDRLAKEKLQEQQSDLEQERRAKEKLQEQQSDLEQDRLAKEKLQEQQSDLEQERRAKEKLQEQQSDLEQERLAKEKLQEQQSDLEQERRAKEKLQEQQSDLEQDRLAKEKLQEQQSDLEQERRAKEKLQEQQSDLEQERRAKEKLQEQQSDLEQERLAKEKLQEQQRDLEQERRAKEKLQEQQSDLEQERRAKEKLQEQQSDLEQERLAKEKLQEQQSDLEQERLAKEKLQEQQSDLEQERLAKEKLQGQQSDLEQERLAKEKLQGQQSDLEQERLAKEKLQEQQSDLEQERLAKEKLQEQQSDLEQERLAKEKLQGQQSDLEQERLAKEKLQGQQSDLEQERLAKEKLQGQQSDLEQERLAKEKLQGQQSDLEQERLAKEKLQEQQSDLEQERLAKEKLQEQQSDLEQERRAKEKLQEQQSDLERTKASKETLQEQQSDLEQERLAKEKLQEQQSDLEQERRAKEKLQEQQSDLEQERLAKEKLQEQQSDLEQERRAKEKLQEQQSDLEQERRAKEKLQEQQSDLEQERRAKEKLQEQQSDLEQERLAKEKLQEQQSDLEQERLAKEKLQEQQSDLEQERRAKEKLQEQQSDLEQERLAKEKLQEQQSDLEQERRAKEKLQEQQSDLEQDRLAKEKLQEQQRDLEQERRAKEKLQEQQSDLEQERRAKEKLQEQQSDLEQERRAKEKLQEQQSDLEQERRAKEKLQEQQSDLEQERLAKEKLQEQQRDLEQERRAKEKLQEQQSDLEQERRAKEKLQEQQSDLEQERLANEKLQEQQRDLEQERRAKEKLQEQQSDLEQERRAKEKLQEQQSDLEQERRAKEKLQEQQSDLEQERLAKEKLQEQQRDLEQERLAKEKLQEQQRDLEQRKADTKKNLERKKEHGDVLAEDLYGRLEIPAIELPSENERGYYIPHQSSLPQDNRGNSRDSKEISIIEKTNRESITTNVEGRRDIHKGHLEEKKDGSIKPEQKEDKSADIQNHTLETVNISDVNDFQISKYEDEISAEYDDSLIDEEEDDEDLDEFKPIVQYDNFQDEENIGIYKELEDLIEKNENLDDLDEGIEKSSEELSEEKIKKGKKYEKTKDNNFKPNDKSLYDEHIKKYKNDKQVNKEKEKFIKSLFHIFDGDNEILQIVDELSEDITKYFMKL

SEQ ID NO: 23 - protein, 475 aa, Artificial Sequence; identical to SEQ ID NO: 13
  Description of Artificial Sequence Synthetic polypeptide

SEQ ID NO: 24 - protein, 559 aa, Plasmodium falciparum
MNHLGNVKYLVIVFLIFFDLFLVNGRDVQNNIVDEIKYREEVCNDEVDLYLLMDCSGSIRRHNWVNHAVPLAMKLIQQLNLNESAIHLYVNIFSNNAKEIIRLHSDASKNKEKALIIIRSLLSTNLPYGRTNLSDALLQVRKHLNDRINRENANQLVVILTDGIPDSIQDSLKESRKLNDRGVKIAVFGIGQGINVAFNRFLVGCHPSDGKCNLYADSAWENVKNVIGPFMKAVCVEVEKTASCGVWDEWSPCSVTCGKGTRSRKREILHEGCTSELQEQCEEERCPPKREPLDVPDEPEDDQPRPRGDNFAVEKPEENIIDNNPQEPSPNPEEGKGENPNGFDLDENPENPPNPDIPQQEPNIPEDSEKEVPSDVPKNPEDDREENFDIPKKPENKHDNQNNLPNDKSDRSIPYSPLPPKVLDNERKQSDPQSQDNNGNRHVPNSEDRETRPHGRNNENRSYNRKYNDTPKHPEREEHEKPDNNKKKGGSDNKYKIAGGIAGGLALLACAGLAYKFVVPGAATPYAGEPAPFDETLGEEDKDLDEPEQFRLPEENEWN

SEQ ID NO: 25 - protein, 536 aa, Artificial Sequence; identical to SEQ ID NO: 15
  Description of Artificial Sequence Synthetic polypeptide

SEQ ID NO: 26 - protein, 237 aa, Listeria monocytogenes
MNAQAEEFKKYLETNGIKPKQFHKKELIFNQWDPQEYCIFLYDGITKLTSISENGTIMNLQYYKGAFVIMSGFIDTETSVGYYNLEVISEQATAYVIKINELKELLSKNLTHFFYVFQTLQKQVSYSLAKFNDFSINGKLGSICGQLLILTYVYGKETPDGIKITLDNLTMQELGYSSGIAHSSAVSRIISKLKQEKVIVYKNSCFYVQNLDYLKRYAPKLDEWFYLACPATWGKLN

SEQ ID NO: 27 - protein, 237 aa, Listeria monocytogenes
MNAQAEEFKKYLETNGIKPKQFHKKELIFNQWDPQEYCIFLYDGITKLTSISENGTIMNLQYYKGAFVIMSGFIDTETSVGYYNLEVISEQATAYVIKINELKELLSKNLTHFFYVFQTLQKQVSYSLAKFNDFSINGKLGSICGQLLILTYVYSKETPDGIKITLDNLTMQELGYSSGIAHSSAVSRIISKLKQEKVIVYKNSCFYVQNLDYLKRYAPKLDEWFYLACPATWGKLN

SEQ ID NO: 28 - protein, 5 aa, Listeria monocytogenes
TEAKD

SEQ ID NO: 29 - protein, 5 aa, Lactococcus lactis
VYADT

SEQ ID NO: 30 - protein, 5 aa, Bacillus anthracis
IQAEV

SEQ ID NO: 31 - protein, 5 aa, Listeria monocytogenes
ASAST

SEQ ID NO: 32 - protein, 5 aa, Listeria monocytogenes
VGAFG

SEQ ID NO: 33 - protein, 5 aa, Bacillus anthracis
AFAED

SEQ ID NO: 34 - protein, 5 aa, Staphylococcus  aureus
VQAAE

SEQ ID NO: 35 - protein, 5 aa, Listeria monocytogenes
DKALT

SEQ ID NO: 36 - protein, 5 aa, Bacillus subtillis
VGAFG

SEQ ID NO: 37 - protein, 100 aa, Artificial Sequence
  Description of Artificial Sequence Synthetic polypeptide
VGLNRFMRAMMVVFITANCITINPDIIFAATDSEDSSLNTDEWEEEKTEEQPSEVNTGPRYETAREVSSRDIEELEKSNKVKNTNKADLIAMLKAKAEKG

SEQ ID NO: 38 - protein, 100 aa, Artificial Sequence
  Description of Artificial Sequence Synthetic polypeptide
MGLNRFMRAMMVVFITANCITINPDIIFAATDSEDSSLNTDEWEEEKTEEQPSEVNTGPRYETAREVSSRDIEELEKSNKVKNTNKADLIAMLKAKAEKG

SEQ ID NO: 39 - protein, 102 aa, Artificial Sequence
  Description of Artificial Sequence Synthetic polypeptide
VGLNRFMRAMMVVFITANCITINPDIIFAATDSEDSSLNTDEWEEEKTEEQPSEVNTGPRYETAREVSSRDIEELEKSNKVKNTNKADLIAMLKAKAEKGGS

SEQ ID NO: 40 - protein, 102 aa, Artificial Sequence
  Description of Artificial Sequence Synthetic polypeptide
MGLNRFMRAMMVVFITANCITINPDIIFAATDSEDSSLNTDEWEEEKTEEQPSEVNTGPRYETAREVSSRDIEELEKSNKVKNTNKADLIAMLKAKAEKGGS

SEQ ID NO: 41 - protein, 29 aa, Listeria monocytogenes
MGLNRFMRAMMVVFITANCITINPDIIFA

SEQ ID NO: 42 - protein, 18 aa, Artificial Sequence
  Description of Artificial Sequence Synthetic peptide
ATDSEDSSLNTDEWEEEK

SEQ ID NO: 43 - protein, 10 aa, Artificial Sequence
  Description of Artificial Sequence Synthetic peptide
ARNDCQEGHI

SEQ ID NO: 44 - RNA, 30 nt, Artificial Sequence
  Description of Artificial Sequence Synthetic oligonucleotide
gcacguaaugauugucaagaaggucauauu

SEQ ID NO: 45 - protein, 10 aa, Artificial Sequence
  Description of Artificial Sequence Synthetic peptide
LKMFPSTWYV

SEQ ID NO: 46 - RNA, 30 nt, Artificial Sequence
  Description of Artificial Sequence Synthetic oligonucleotide
uuaaaaauguuuccaaguacaugguauguu

SEQ ID NO: 47 - protein, 8 aa, Artificial Sequence
  Description of Artificial Sequence Synthetic peptide
KYGVSQDI

SEQ ID NO: 48 - protein, 9 aa, Artificial Sequence
  Description of Artificial Sequence Synthetic peptide
GYKDGNEYI

SEQ ID NO: 49 - protein, 12 aa, Artificial Sequence
  Description of Artificial Sequence Synthetic peptide
NEKYAQAYPNVS

SEQ ID NO: 50 - protein, 9 aa, Artificial Sequence
  Description of Artificial Sequence Synthetic peptide
YYIPHQSSL

SEQ ID NO: 51 - protein, 9 aa, Artificial Sequence
  Description of Artificial Sequence Synthetic peptide
NYDNAGTNL

SEQ ID NO: 52 - protein, 9 aa, Artificial Sequence
  Description of Artificial Sequence Synthetic peptide
SYIPSAEKI

SEQ ID NO: 53 - protein, 9 aa, Artificial Sequence
  Description of Artificial Sequence Synthetic peptide
RAHYNIVTF

SEQ ID NO: 54 - protein, 15 aa, Artificial Sequence
  Description of Artificial Sequence Synthetic peptide
VAENVKPPKVDPATY

SEQ ID NO: 55 - protein, 15 aa, Artificial Sequence
  Description of Artificial Sequence Synthetic peptide
VKPPKVDPATYGIIV

SEQ ID NO: 56 - protein, 15 aa, Artificial Sequence
  Description of Artificial Sequence Synthetic peptide
VSDEIWNYNSPDVSE

SEQ ID NO: 57 - protein, 15 aa, Artificial Sequence
  Description of Artificial Sequence Synthetic peptide
IWNYNSPDVSESEES

SEQ ID NO: 58 - protein, 17 aa, Artificial Sequence
  Description of Artificial Sequence Synthetic peptide
ERLAKEKLQEQQRDLEQ

SEQ ID NO: 59 - protein, 17 aa, Artificial Sequence
  Description of Artificial Sequence Synthetic peptide
EQQSDLEQERLAKEKLQ

SEQ ID NO: 60 - protein, 8 aa, Artificial Sequence
  Description of Artificial Sequence Synthetic peptide
DPNANPNV

SEQ ID NO: 61 - protein, 4 aa, Artificial Sequence
  Description of Artificial Sequence Synthetic peptide
NANP

SEQ ID NO: 62 - protein, 478 aa, Artificial Sequence
  Description of Artificial Sequence Synthetic polypeptide
MGTNSEKDEIIKSNLRSGSSNSRNRINEEKHEKKHVLSHNSYEKTKNNENNKFFDKDKELTMSNVKNVSQTNFKSLLRNLGVSENIFLKENKLNKEGKLIEHIINDDDDKKKYIKGQDENRQEDLEEKAAEQQSDLEQERLAKEKLQEQQSDLEQERLAKEKLQERLAKEKLQEQQRDLEQERLAKEKLQEQQRDLEQRKADTKKNLERKKEHGDVLAEDLYGRLEIPAIELPSENERGYYIPHQSSLPQDNRGNSRDSKEISIIEKTNRESITTNVEGRRDIHKGHLEEKKDGSIKPEQKEDKSADIQNHTLETVNISDVNDFQISKYEDEISAEYDDSLIDEEEDDEDLDEFKPIVQYDNFQDEENIGIYKELEDLIEKNENLDDLDEGIEKSSEELSEEKIKKGKKYEKTKDNNFKPNDKSLYDEHIKKYKNDKQVNKEKEKFIKSLFHIFDGDNEILQIVDELSEDITKYFMKL

(19) United States
(12) Patent Application Publication
(10) Pub. No.: US 2013/0323275 A1
(43) Pub. Date: Dec. 5, 2013

(54) METHODS AND COMPOSITIONS FOR INDUCING A T-CELL RESPONSE TO PLASMODIUM SPECIES

(71) Applicants: Peter M. Lauer, Albany, CA (US); Dirk G. Brockstedt, Richmond, CA (US); Thomas W. Dubensky, Piedmont, CA (US)

(72) Inventors: Peter M. Lauer, Albany, CA (US); Dirk G. Brockstedt, Richmond, CA (US); Thomas W. Dubensky, Piedmont, CA (US)

(73) Assignee: ADURO BIOTECH, Berkeley, CA (US)

(21) Appl. No.: 13/878,494

(22) PCT Filed: Oct. 10, 2011
(86) PCT No.: PCT/US11/55568

§ 371 Date: May 23, 2013

Related U.S. Application Data
(60) Provisional application No. 61/391,650, filed on Oct. 10, 2010.

(51) Int. Cl. A61K 39/002; C12N 15/74

(52) U.S. Cl. CPC A61K 39/002; C12N 15/74
USPC 424/191.1; 435/252.3

(57) ABSTRACT

The present invention relates to methods of inducing a T-cell response against a Plasmodium species antigen in a subject. These method comprise administering to a subject a composition comprising a bacterium which expresses one or more immunogenic polypeptides, the amino acid sequence of which comprise one or more amino acid sequences derived from wild-type Plasmodium LSA1, Ce1TOS, CSP, and/or TRAP sequences, wherein said amino acid sequences are derived by (i) codon optimization of the wild-type sequence for expression in said bacterium, (ii) deletion of at least one hydrophobic region present in the wild-type sequence, and/or (iii) in the case of LSA1 and CSP, minimization of repeat units present in the wild-type sequence.

### BACKGROUND OF THE INVENTION

The present invention claims priority from U.S. Provisional Patent Application No. 61/391,650, filed Oct. 10, 2010, which is hereby incorporated in its entirety, including all tables, figures and claims.

The following discussion of the background of the invention is merely provided to aid the reader in understanding the invention and is not admitted to describe or constitute prior art to the present invention.

Malaria is a major infectious disease, affecting 500 million people and causing 2.7 million deaths each year. The severity of malaria is, in part, due to the failure of the host immune system to effectively clear an infection and generate protective immunity. Dendritic cells (DCs) present components of pathogens to circulating T cells, thereby initiating a highly specific immune response to clear an infection. It has been reported, however, that DCs are modified by malaria parasites, resulting in inefficient priming of the adaptive immune system. See, e.g., Millington et al., PLoS Pathog. 3(10): e143. doi:10.1371/journal.ppat.0030143. As a result, T-cell function and migration are suppressed, with deleterious effects on both cell-mediated and humoral responses to Plasmodium infection.

There remains a need in the art for compositions and methods for stimulating an effective immune response to Plasmodium species.

### BRIEF SUMMARY OF THE INVENTION

The present invention provides compositions and methods for delivery of one or more Plasmodium antigens using a bacterium recombinantly encoding and expressing such antigens.

In a first aspect of the invention, the invention relates to methods of inducing a T-cell response against a Plasmodium species antigen in a subject. These method comprise administering to a subject a composition comprising a bacterium which expresses one or more immunogenic polypeptides, the amino acid sequence of which comprise one or more amino acid sequences derived from wild-type Plasmodium LSA1 (liver-stage antigen 1), Ce1TOS, CSP (circumsporozoite protein), and/or TRAP (Thrombospondin-related adhesive protein, which is also known as sporozoite surface protein 2 or SSP2) sequences, wherein said amino acid sequences are derived by (i) codon optimization of the wild-type sequence for expression in said bacterium, (ii) deletion of at least one hydrophobic region present in the wild-type sequence, and/or (iii) in the case of LSA1 and CSP, minimization of repeat units present in the wild-type sequence.

As described herein, such methods can stimulate an antigen-specific T cell (CD4+ and/or CD8+) response in said subject to the recombinantly expressed immunogenic Plasmodium polypeptides. Preferably, when delivered to the subject, the compositions of the present invention induce an increase in the serum concentration of one or more, and preferably each of, proteins selected from the group consisting of IL-12p70, IFN-γ, IL-6, TNF α, and MCP-1 at 24 hours following said delivery; and induce a CD4+ and/or CD8+ antigen-specific T cell response against one or more of said immunogenic Plasmodium antigen polypeptide(s) expressed by the bacterium.

In a related aspect of the invention, the invention relates to compositions useful for inducing a T-cell response a Plasmodium species in a subject. Such compositions comprise a bacterium which comprises a nucleic acid molecule, the sequence of which encodes one or more immunogenic polypeptides, the amino acid sequence of which comprise one or more amino acid sequences derived from wild-type Plasmodium LSA1, Ce1TOS, CSP, and/or TRAP sequences, wherein said amino acid sequences are derived by (i) codon optimization of the wild-type sequence for expression in said bacterium, (ii) deletion of at least one hydrophobic region present in the wild-type sequence, and/or (iii) in the case of LSA1 and CSP, minimization of repeat units present in the wild-type sequence.

In another related aspect, the invention relates to a isolated nucleic acid molecule, the sequence of which encodes one or more immunogenic polypeptides, the amino acid sequence of which comprise one or more amino acid sequences derived from wild-type Plasmodium LSA1, Ce1TOS, CSP, and/or TRAP sequences, wherein said amino acid sequences are derived by (i) codon optimization of the wild-type sequence for expression in said bacterium, (ii) deletion of at least one hydrophobic region present in the wild-type sequence, and/or (iii) in the case of LSA1 and CSP, minimization of repeat units present in the wild-type sequence.

Methods for deriving appropriate immunogenic polypeptide sequences are described in detail hereinafter, and exemplary immunogenic polypeptide sequences derived from Plasmodium falciparum LSA1, Ce1TOS, CSP, and TRAP are provided. Selection methods can comprise the selection of LSA1, Ce1TOS, CSP, and/or TRAP amino acid sequences having no region of hydrophobicity that exceeds 50% of the peak hydrophobicity of Listeria ActA-N100 and which are predicted to encode one or more MHC class I epitopes. The ability of such polypeptides to generate a CD4+ and/or CD8+ T cell response may be confirmed by a variety of methods described in detail herein and that are well known in the art.

In certain embodiments, the immunogenic polypeptide(s) comprise one or more amino acid sequences selected from the group consisting of SEQ ID NOS: 7, 9, 11, 13, 15, and 17; or modifications or fragments thereof sharing at least 90% identity with at least 30 amino acids from these sequences. In various embodiments, the nucleic acid encoding such immunogenic polypeptide(s) comprise one or more nucleic acid sequences selected from the group consisting of SEQ ID NOS: 6, 8, 10, 12, 14, and 16; or modifications or fragments thereof sharing at least 90% identity with at least 90 residues from these sequences.

Numerous Plasmodium species may serve as the source materials for the antigen polypeptide(s), and the corresponding amino acids, of the present invention. Five species of the plasmodium parasite can infect humans: the most serious forms of the disease are caused by Plasmodium falciparum, and is thus preferred. However, Plasmodium vivax, Plasmodium ovale and Plasmodium malariae cause disease in humans, albeit a disease that is not generally fatal. A fifth species, Plasmodium knowlesi, is a zoonosis that causes malaria in macaques but can also infect humans.

A number of bacterial species have been developed for use as vaccines and can be used as a vaccine platform in present invention, including, but not limited to, Shigella flexneri, Escherichia coli, Listeria monocytogenes, Yersinia enterocolitica, Salmonella typhimurium, Salmonella typhi or mycobacterium species. This list is not meant to be limiting. The present invention contemplates the use of attenuated, commensal, and/or killed but metabolically active bacterial strains as vaccine platforms. In preferred embodiments the bacterium is Listeria monocytogenes comprising a nucleic acid sequence encoding for expression by the bacterium one or more immunogenic Plasmodium-derived antigen polypeptides of the invention. This nucleic acid is most preferably integrated into the genome of the bacterium. Attenuated and killed but metabolically active forms of Listeria monocytogenes are particularly preferred, and Listeria monocytogenes harboring an attenuating mutation in actA and/or inlB is described hereinafter in preferred embodiments.

The vaccine compositions described herein can be administered to a host, either alone or in combination with a pharmaceutically acceptable excipient, in an amount sufficient to induce an appropriate immune response to prevent or treat a Plasmodium infection. Preferred conditions selected to induce a T cell response in a subject comprise administering the vaccine platform intravenously to a subject; however, administration may be oral, intravenous, subcutaneous, dermal, intradermal, intramuscular, mucosal, parenteral, intraorgan, intralesional, intranasal, inhalation, intraocular, intravascular, intranodal, by scarification, rectal, intraperitoneal, or any one or combination of a variety of well-known routes of administration.

In certain preferred embodiments, after the subject has been administered an effective dose of a vaccine containing the immunogenic polypeptides to prime the immune response, a second vaccine is administered. This is referred to in the art as a “prime-boost” regimen. In such a regimen, the compositions and methods of the present invention may be used as the “prime” delivery, as the “boost” delivery, or as both a “prime” and a “boost.” Examples of such regimens are described hereinafter.

A preferred Listeria monocytogenes for use in the present invention comprises a mutation in the prfA gene which locks the expressed prfA transcription factor into a constitutively active state. For example, a PrfA* mutant (G155S) has been shown to enhance functional cellular immunity following a prime-boost intravenous or intramuscular immunization regimen.

In certain embodiments, the immunogenic polypeptide(s) of the present invention are expressed as one or more fusion proteins comprising an in frame secretory signal sequence, thereby resulting in their secretion as soluble polypeptide(s) by the bacterium. Numerous exemplary signal sequences are known in the art for use in bacterial expression systems. In the case where the bacterium is Listeria monocytogenes, it is preferred that the secretory signal sequence is a Listeria monocytogenes signal sequence, most preferably the ActA signal sequence. Additional ActA or other linker amino acids may also be expressed fused to the immunogenic polypeptide(s). In preferred embodiments, one or more immunogenic polypeptide(s) are expressed as fusion protein(s) comprising an in frame ActA-N100 sequence (e.g., selected from the group consisting of SEQ ID NO: 37, 38 and 39) or an amino acid sequence having at least 90% sequence identity to said ActA-N100 sequence.

In preferred embodiments, the vaccine composition comprises a Listeria monocytogenes expressing a fusion protein comprising:
(a) an ActA-N100 sequence selected from the group consisting of SEQ ID NO: 37, 38 and 39, or an amino acid sequence having at least 90% sequence identity to such a ActA-N100 sequence; and
(b) an amino acid sequence selected from the group consisting of SEQ ID NOS: 7, 9, 11, 13, 15, and 17, or a modification or fragment thereof sharing at least 90% identity with at least 30 amino acids from one of these sequences,
wherein the fusion protein is expressed from a nucleic acid sequence operably linked to a Listeria monocytogenes ActA promoter.

As noted above, in certain embodiments the nucleic acid sequences encoding the antigenic polypeptide(s) are codon optimized for expression by the bacterium (e.g., Listeria monocytogenes). As described hereinafter, different organisms often display “codon bias”; that is, the degree to which a given codon encoding a particular amino acid appears in the genetic code varies significantly between organisms. In general, the more rare codons that a gene contains, the less likely it is that the heterologous protein will be expressed at a reasonable level within that specific host system. These levels become even lower if the rare codons appear in clusters or in the N-terminal portion of the protein. Replacing rare codons with others that more closely reflect the host system's codon bias without modifying the amino acid sequence can increase the levels of functional protein expression. Methods for codon optimization are described hereinafter.

It is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE FIGURES

FIG. 1. Schematic diagram of LSA1 fusion proteins secreted from Listeria vaccine strains. The synthetic LSA1 coding sequence was fused in-frame at the amino terminus with the ActAN100 coding sequence and at the carboxy terminus with the SL8 tag. Minimized repeat sequences are noted as well as the H-2K^{d} T cell epitope used in immunogenicity studies. A Kyte-Doolittle plot is shown with the full length construct.

FIG. 2. Schematic diagram of Ce1TOS fusion proteins secreted from Listeria vaccine strains. The synthetic Ce1TOS coding sequence was fused in-frame at the amino terminus with the ActAN100 coding sequence and at the carboxy terminus with the SL8 tag. A Kyte-Doolittle plot is shown with the full length construct.

FIG. 3. Schematic diagram of CSP fusion proteins secreted from Listeria vaccine strains. The synthetic CSP coding sequence was fused in-frame at the amino terminus with the ActAN100 coding sequence and at the carboxy terminus with the SL8 tag. Minimized repeat sequences are noted. The H-2K^{d} T cell epitope used in immunogenicity studies and T* epitope region from human immunology studies are shown. A Kyte-Doolittle plot is shown with the full length construct.

FIG. 4. Schematic diagram of TRAP fusion proteins secreted from Listeria vaccine strains. The synthetic TRAP coding sequence was fused in-frame at the amino terminus with the ActAN100 coding sequence and at the carboxy terminus with the SL8 tag. A Kyte-Doolittle plot is shown with the full length construct.

FIG. 5. B3Z T cell hybridoma activation profiles of LSA1, Ce1TOS, and CSP constructs from infected mouse dendritic cells. The constructs shown in FIGS. 1-3 were tested for SIINFEKL presentation and beta-galactosidase activation, a measure of in vitro T cell activation. Top panel: vaccine candidates in the live attenuated Listeria strain background. Bottom panel: vaccine candidates in the KBMA Listeria strain background. The most effective activators were the full length LSA1 construct, the full length Ce1TOS construct, and the CSP construct that included aa1-224 (FIGS. 1-3).

FIG. 6. B3Z T cell hybridoma activation profiles of TRAP constructs and bivalent vaccine strains from infected mouse dendritic cells. Top panel: TRAP constructs (FIG. 4) were tested for SIINFEKL presentation and beta-galactosidase activation. The most effective activator was TRAP(24-497). Bottom panel: bi-valent vaccine constructs were confirmed for B3Z activation.

FIG. 7. Expression and secretion of encoded malaria antigens (CSP, LSA1, and Ce1TOS) in DC2.4 cells infected with candidate Lm vaccine strains. Left panel: Full-length antigens and high-expressing and low-expressing controls; Right panel: Antigen sub-fragments with deleted hydrophobic regions. Gel symbols: (*), malaria antigens; (>), high antigen expressing control; (>>), low-expressing antigen control. Strains bolded in red text (BH2202, BH2200, and BH2210) are high-expressing malaria antigens.

FIG. 8. Expression and secretion of encoded malaria antigens (TRAP and bivalent candidates) in DC2.4 cells infected with Lm vaccine strains. Left panel: Expression of various TRAP vaccine constructs; Right panel: Expression from candidate bivalent strains expressing single antigens from distinct loci (tRNA^{Arg} or comK as noted in table at bottom right).

FIG. 9. Expression and secretion of candidate bivalent and trivalent vaccine candidates in DC2.4 cells. Expression from bivalent strains expressing two antigens (Ag2-CSP or CSP-Ag2) as fusion proteins (lanes 3 and 4), or trivalent strains encoding a combination of Ag2-CSP or CSP-Ag2 fusion proteins at one genomic locus together with expression of LSA-1 from a distinct locus (lanes 5 and 6).

FIG. 10. Primary surrogate immunogenicity of vaccine strain candidates in C57BL/6 mice. Female C57BL/6 mice were vaccinated IV with 5×10⁶ cfu of the respective vaccine strain. OVA-specific CD8+ T cell immunity was determined by intracellular cytokine staining (ICS) or ELISPOT on day 7, the peak of the primary response. (A) Top: Vaccine strains for LSA1, Ce1TOS, and CSP; Bottom: splenic SL8 immunogenicity for each strain measured by ICS, unstimulated (left) and stimulated (right); (B) Top: Vaccine strains for TRAP, using Ce1TOS as a positive control; Bottom: splenic SL8 immunogenicity for each strain, unstimulated (left) and stimulated (right) as measured by ELISPOT.

FIG. 11. Primary CSP- or LSA-1-specific T cell responses were determined in spleen and liver by ICS at the peak of the primary response. Top panel: CS-specific CD8+ T cell responses in spleen and liver; Bottom panel: LSA-1-specific CD8+ T cell responses in spleen and liver.

FIG. 12. CSP- or LSA-1-specific T cell responses were determined in spleen and liver by ICS at the peak of the primary and secondary response. Hepatic T cell responses were determined in the presence or absence of P815 cells as antigen presenting cells. Top panel: CS-specific CD8+ T cell responses in spleen and liver; Bottom panel: LSA-1-specific CD8+ T cell responses in spleen and liver.

FIG. 13. Ce1TOS specific T cell response following one or two vaccinations in C57BL/6 mice. Ce1TOS-specific T cell responses were determined in spleen and liver by ICS at the peak of the primary and secondary response. Hepatic T cell responses were determined in the presence or absence of EL-4 cells as antigen presenting cells. Left panel: CD4+ T cell responses in the spleen; Right panel: CD4+ T cell responses in the liver.

FIG. 14. Immunogenicity of Lm-Pf Ag monovalent and bivalent vaccine strains. Balb/c mice were vaccinated once IV with 2×10⁶ cfu of the monovalent Lm vaccine strains encoding either the CS protein (BH2224) or LSA-1 (BH2214) or the bivalent vaccine strain encoding both, CSP and LSA-1 (BH2370). (A) CD8+ T cell response specific to CS; (B) CD8+ T cell response specific to LSA-1.

FIG. 15. Immunogenicity of Lm-Pf Ag monovalent and trivalent vaccine strains. Top panel: Balb/c mice were vaccinated once IV with 2×10⁶ cfu of the monovalent Lm vaccine strains encoding either the CS protein (BH2224) or LSA-1 (BH2214) or the trivalent vaccine strain encoding CSP, LSA-1, and Ce1TOS (BH2448). Bottom panel: C57BL/6 mice were vaccinated once IV with 2×10⁶ cfu of the monovalent Lm vaccine strains encoding Ce1TOS (BH2216) or the trivalent vaccine strain encoding CSP, LSA-1, and Ce1TOS (BH2448).

### DETAILED DESCRIPTION OF THE INVENTION

The present invention relates to compositions and methods for delivery of prophylaxis or immunotherapy using a bacterium encoding and expressing one or more T-cell antigens derived from a Plasmodium species which causes human or animal disease.

It is to be understood that the invention is not limited in its application to the details of construction and to the arrangements of the components set forth in the following description or illustrated in the drawings. The invention is capable of embodiments in addition to those described and of being practiced and carried out in various ways. Also, it is to be understood that the phraseology and terminology employed herein, as well as the abstract, are for the purpose of description and should not be regarded as limiting.

As such, those skilled in the art will appreciate that the conception upon which this disclosure is based may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

1. Definitions

Abbreviations used to indicate a mutation in a gene, or a mutation in a bacterium comprising the gene, are as follows. By way of example, the abbreviation “L. monocytogenes ΔactA” means that part, or all, of the actA gene was deleted. The delta symbol (Δ) means deletion. An abbreviation including a superscripted minus sign (Listeria ActA⁻) means that the actA gene was mutated, e.g., by way of a deletion, point mutation, or frameshift mutation, but not limited to these types of mutations.

“Administration” as it applies to a human, mammal, mammalian subject, animal, veterinary subject, placebo subject, research subject, experimental subject, cell, tissue, organ, or biological fluid, refers without limitation to contact of an exogenous ligand, reagent, placebo, small molecule, pharmaceutical agent, therapeutic agent, diagnostic agent, or composition to the subject, cell, tissue, organ, or biological fluid, and the like. “Administration” can refer, e.g., to therapeutic, pharmacokinetic, diagnostic, research, placebo, and experimental methods. Treatment of a cell encompasses contact of a reagent to the cell, as well as contact of a reagent to a fluid, where the fluid is in contact with the cell. “Administration” also encompasses in vitro and ex vivo treatments, e.g., of a cell, by a reagent, diagnostic, binding composition, or by another cell.

An “agonist,” as it relates to a ligand and receptor, comprises a molecule, combination of molecules, a complex, or a combination of reagents, that stimulates the receptor. For example, an agonist of granulocyte-macrophage colony stimulating factor (GM-CSF) can encompass GM-CSF, a mutein or derivative of GM-CSF, a peptide mimetic of GM-CSF, a small molecule that mimics the biological function of GM-CSF, or an antibody that stimulates GM-CSF receptor.

An “antagonist,” as it relates to a ligand and receptor, comprises a molecule, combination of molecules, or a complex, that inhibits, counteracts, downregulates, and/or desensitizes the receptor. “Antagonist” encompasses any reagent that inhibits a constitutive activity of the receptor. A constitutive activity is one that is manifest in the absence of a ligand/receptor interaction. “Antagonist” also encompasses any reagent that inhibits or prevents a stimulated (or regulated) activity of a receptor. By way of example, an antagonist of GM-CSF receptor includes, without implying any limitation, an antibody that binds to the ligand (GM-CSF) and prevents it from binding to the receptor, or an antibody that binds to the receptor and prevents the ligand from binding to the receptor, or where the antibody locks the receptor in an inactive conformation.

As used herein, an “analog” or “derivative” with reference to a peptide, polypeptide or protein refers to another peptide, polypeptide or protein that possesses a similar or identical function as the original peptide, polypeptide or protein, but does not necessarily comprise a similar or identical amino acid sequence or structure of the original peptide, polypeptide or protein. An analog preferably satisfies at least one of the following: (a) a proteinaceous agent having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the original amino acid sequence (b) a proteinaceous agent encoded by a nucleotide sequence that hybridizes under stringent conditions to a nucleotide sequence encoding the original amino acid sequence; and (c) a proteinaceous agent encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the nucleotide sequence encoding the original amino acid sequence.

“Antigen presenting cells” (APCs) are cells of the immune system used for presenting antigen to T cells. APCs include dendritic cells, monocytes, macrophages, marginal zone Kupffer cells, microglia, Langerhans cells, T cells, and B cells. Dendritic cells occur in at least two lineages. The first lineage encompasses pre-DC1, myeloid DC1, and mature DC1. The second lineage encompasses CD34⁺CD45RA⁻ early progenitor multipotent cells, CD34⁺CD45RA⁺ cells, CD34⁺CD45RA⁺CD4⁺ IL-3Rα⁺ pro-DC2 cells, CD4⁺CD11⁻ plasmacytoid pre-DC2 cells, lymphoid human DC2 plasmacytoid-derived DC2s, and mature DC2s.

“Attenuation” and “attenuated” encompasses a bacterium, virus, parasite, infectious organism, prion, tumor cell, gene in the infectious organism, and the like, that is modified to reduce toxicity to a host. The host can be a human or animal host, or an organ, tissue, or cell. The bacterium, to give a non-limiting example, can be attenuated to reduce binding to a host cell, to reduce spread from one host cell to another host cell, to reduce extracellular growth, or to reduce intracellular growth in a host cell. Attenuation can be assessed by measuring, e.g., an indicum or indicia of toxicity, the LD₅₀, the rate of clearance from an organ, or the competitive index (see, e.g., Auerbuch, et al. (2001) Infect. Immunity 69:5953-5957). Generally, an attenuation results an increase in the LD₅₀ and/or an increase in the rate of clearance by at least 25%; more generally by at least 50%; most generally by at least 100% (2-fold); normally by at least 5-fold; more normally by at least 10-fold; most normally by at least 50-fold; often by at least 100-fold; more often by at least 500-fold; and most often by at least 1000-fold; usually by at least 5000-fold; more usually by at least 10,000-fold; and most usually by at least 50,000-fold; and most often by at least 100,000-fold.

“Attenuated gene” encompasses a gene that mediates toxicity, pathology, or virulence, to a host, growth within the host, or survival within the host, where the gene is mutated in a way that mitigates, reduces, or eliminates the toxicity, pathology, or virulence. The reduction or elimination can be assessed by comparing the virulence or toxicity mediated by the mutated gene with that mediated by the non-mutated (or parent) gene. “Mutated gene” encompasses deletions, point mutations, and frameshift mutations in regulatory regions of the gene, coding regions of the gene, non-coding regions of the gene, or any combination thereof.

“Conservatively modified variants” applies to both amino acid and nucleic acid sequences. With respect to particular nucleic acid sequences, a conservatively modified variant refers to nucleic acids encoding identical amino acid sequences, or amino acid sequences that have one or more conservative substitutions. An example of a conservative substitution is the exchange of an amino acid in one of the following groups for another amino acid of the same group (U.S. Pat. No. 5,767,063 issued to Lee, et al.; Kyte and Doolittle (1982) J. Mol. Biol. 157:105-132).
(1) Hydrophobic: Norleucine, Ile, Val, Leu, Phe, Cys, Met;
(2) Neutral hydrophilic: Cys, Ser, Thr;
(3) Acidic: Asp, Glu;
(4) Basic: Asn, Gln, His, Lys, Arg;
(5) Residues that influence chain orientation: Gly, Pro;
(6) Aromatic: Trp, Tyr, Phe; and
(7) Small amino acids: Gly, Ala, Ser.

“Effective amount” encompasses, without limitation, an amount that can ameliorate, reverse, mitigate, prevent, or diagnose a symptom or sign of a medical condition or disorder. Unless dictated otherwise, explicitly or by context, an “effective amount” is not limited to a minimal amount sufficient to ameliorate a condition.

An “extracellular fluid” encompasses, e.g., serum, plasma, blood, interstitial fluid, cerebrospinal fluid, secreted fluids, lymph, bile, sweat, fecal matter, and urine. An “extracelluar fluid” can comprise a colloid or a suspension, e.g., whole blood or coagulated blood.

The term “fragments” in the context of polypeptides include a peptide or polypeptide comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, at least 150 contiguous amino acid residues, at least 175 contiguous amino acid residues, at least 200 contiguous amino acid residues, or at least 250 contiguous amino acid residues of the amino acid sequence of a larger polypeptide.

“Gene” refers to a nucleic acid sequence encoding an oligopeptide or polypeptide. The oligopeptide or polypeptide can be biologically active, antigenically active, biologically inactive, or antigenically inactive, and the like. The term gene encompasses, e.g., the sum of the open reading frames (ORFs) encoding a specific oligopeptide or polypeptide; the sum of the ORFs plus the nucleic acids encoding introns; the sum of the ORFs and the operably linked promoter(s); the sum of the ORFS and the operably linked promoter(s) and any introns; the sum of the ORFS and the operably linked promoter(s), intron(s), and promoter(s), and other regulatory elements, such as enhancer(s). In certain embodiments, “gene” encompasses any sequences required in cis for regulating expression of the gene. The term gene can also refer to a nucleic acid that encodes a peptide encompassing an antigen or an antigenically active fragment of a peptide, oligopeptide, polypeptide, or protein. The term gene does not necessarily imply that the encoded peptide or protein has any biological activity, or even that the peptide or protein is antigenically active. A nucleic acid sequence encoding a non-expressable sequence is generally considered a pseudogene. The term gene also encompasses nucleic acid sequences encoding a ribonucleic acid such as rRNA, tRNA, or a ribozyme.

“Growth” of a bacterium such as Listeria encompasses, without limitation, functions of bacterial physiology and genes relating to colonization, replication, increase in protein content, and/or increase in lipid content. Unless specified otherwise explicitly or by context, growth of a Listeria encompasses growth of the bacterium outside a host cell, and also growth inside a host cell. Growth related genes include, without implying any limitation, those that mediate energy production (e.g., glycolysis, Krebs cycle, cytochromes), anabolism and/or catabolism of amino acids, sugars, lipids, minerals, purines, and pyrimidines, nutrient transport, transcription, translation, and/or replication. In some embodiments, “growth” of a Listeria bacterium refers to intracellular growth of the Listeria bacterium, that is, growth inside a host cell such as a mammalian cell. While intracellular growth of a Listeria bacterium can be measured by light microscopy or colony forming unit (CFU) assays, growth is not to be limited by any technique of measurement. Biochemical parameters such as the quantity of a listerial antigen, listerial nucleic acid sequence, or lipid specific to the Listeria bacterium, can be used to assess growth. In some embodiments, a gene that mediates growth is one that specifically mediates intracellular growth. In some embodiments, a gene that specifically mediates intracellular growth encompasses, but is not limited to, a gene where inactivation of the gene reduces the rate of intracellular growth but does not detectably, substantially, or appreciably, reduce the rate of extracellular growth (e.g., growth in broth), or a gene where inactivation of the gene reduces the rate of intracellular growth to a greater extent than it reduces the rate of extracellular growth. To provide a non-limiting example, in some embodiments, a gene where inactivation reduces the rate of intracellular growth to a greater extent than extracellular growth encompasses the situation where inactivation reduces intracellular growth to less than 50% the normal or maximal value, but reduces extracellular growth to only 1-5%, 5-10%, or 10-15% the maximal value. The invention, in certain aspects, encompasses a Listeria attenuated in intracellular growth but not attenuated in extracellular growth, a Listeria not attenuated in intracellular growth and not attenuated in extracellular growth, as well as a Listeria not attenuated in intracellular growth but attenuated in extracellular growth.

A “hydropathy analysis” refers to the analysis of a polypeptide sequence by the method of Kyte and Doolittle: “A Simple Method for Displaying the Hydropathic Character of a Protein”. J. Mol. Biol. 157(1982)105-132. In this method, each amino acid is given a hydrophobicity score between 4.6 and −4.6. A score of 4.6 is the most hydrophobic and a score of −4.6 is the most hydrophilic. Then a window size is set. A window size is the number of amino acids whose hydrophobicity scores will be averaged and assigned to the first amino acid in the window. The calculation starts with the first window of amino acids and calculates the average of all the hydrophobicity scores in that window. Then the window moves down one amino acid and calculates the average of all the hydrophobicity scores in the second window. This pattern continues to the end of the protein, computing the average score for each window and assigning it to the first amino acid in the window. The averages are then plotted on a graph. The y axis represents the hydrophobicity scores and the x axis represents the window number. The following hydrophobicity scores are used for the 20 common amino acids.

| | |
|---|---|
| Arg: | −4.5 |
| Thr: | −0.7 |
| Asp: | −3.5 |
| Met: | 1.9 |
| His: | −3.2 |
| Leu: | 3.8 |
| Trp: | −0.9 |
| Ser: | −0.8 |
| Asn: | −3.5 |
| Ala: | 1.8 |
| Glu: | −3.5 |
| Phe: | 2.8 |
| Tyr: | −1.3 |
| Ile: | 4.5 |
| Lys: | −3.9 |
| Gly: | −0.4 |
| Gln: | −3.5 |
| Cys: | 2.5 |
| Pro: | −1.6 |
| Val: | 4.2 |

A composition that is “labeled” is detectable, either directly or indirectly, by spectroscopic, photochemical, biochemical, immunochemical, isotopic, or chemical methods. For example, useful labels include ³²P, ³³P, ³⁵S, ¹⁴C, ³H, ¹²⁵I, stable isotopes, epitope tags, fluorescent dyes, electron-dense reagents, substrates, or enzymes, e.g., as used in enzyme-linked immunoassays, or fluorettes (see, e.g., Rozinov and Nolan (1998) Chem. Biol. 5:713-728).

“Ligand” refers to a small molecule, peptide, polypeptide, or membrane associated or membrane-bound molecule, that is an agonist or antagonist of a receptor. “Ligand” also encompasses a binding agent that is not an agonist or antagonist, and has no agonist or antagonist properties. By convention, where a ligand is membrane-bound on a first cell, the receptor usually occurs on a second cell. The second cell may have the same identity (the same name), or it may have a different identity (a different name), as the first cell. A ligand or receptor may be entirely intracellular, that is, it may reside in the cytosol, nucleus, or in some other intracellular compartment. The ligand or receptor may change its location, e.g., from an intracellular compartment to the outer face of the plasma membrane. The complex of a ligand and receptor is termed a “ligand receptor complex.” Where a ligand and receptor are involved in a signaling pathway, the ligand occurs at an upstream position and the receptor occurs at a downstream position of the signaling pathway.

“Nucleic acid” refers to deoxyribonucleotides or ribonucleotides and polymers thereof in either single stranded, double-stranded form, or multi-stranded form. Non-limiting examples of a nucleic acid are a, e.g., cDNA, mRNA, oligonucleotide, and polynucleotide. A particular nucleic acid sequence can also implicitly encompasses “allelic variants” and “splice variants.”

“Operably linked” in the context of a promoter and a nucleic acid encoding a mRNA means that the promoter can be used to initiate transcription of that nucleic acid.

The terms “percent sequence identity” and “% sequence identity” refer to the percentage of sequence similarity found by a comparison or alignment of two or more amino acid or nucleic acid sequences. Percent identity can be determined by a direct comparison of the sequence information between two molecules by aligning the sequences, counting the exact number of matches between the two aligned sequences, dividing by the length of the shorter sequence, and multiplying the result by 100. An algorithm for calculating percent identity is the Smith-Waterman homology search algorithm (see, e.g., Kann and Goldstein (2002) Proteins 48:367-376; Arslan, et al. (2001) Bioinformatics 17:327-337).

By “purified” and “isolated” is meant, when referring to a polypeptide, that the polypeptide is present in the substantial absence of the other biological macromolecules with which it is associated in nature. The term “purified” as used herein means that an identified polypeptide often accounts for at least 50%, more often accounts for at least 60%, typically accounts for at least 70%, more typically accounts for at least 75%, most typically accounts for at least 80%, usually accounts for at least 85%, more usually accounts for at least 90%, most usually accounts for at least 95%, and conventionally accounts for at least 98% by weight, or greater, of the polypeptides present. The weights of water, buffers, salts, detergents, reductants, protease inhibitors, stabilizers (including an added protein such as albumin), and excipients, and molecules having a molecular weight of less than 1000, are generally not used in the determination of polypeptide purity. See, e.g., discussion of purity in U.S. Pat. No. 6,090,611 issued to Covacci, et al.

“Peptide” refers to a short sequence of amino acids, where the amino acids are connected to each other by peptide bonds. A peptide may occur free or bound to another moiety, such as a macromolecule, lipid, oligo- or polysaccharide, and/or a polypeptide. Where a peptide is incorporated into a polypeptide chain, the term “peptide” may still be used to refer specifically to the short sequence of amino acids. A “peptide” may be connected to another moiety by way of a peptide bond or some other type of linkage. A peptide is at least two amino acids in length and generally less than about 25 amino acids in length, where the maximal length is a function of custom or context. The terms “peptide” and “oligopeptide” may be used interchangeably.

“Protein” generally refers to the sequence of amino acids comprising a polypeptide chain. Protein may also refer to a three dimensional structure of the polypeptide. “Denatured protein” refers to a partially denatured polypeptide, having some residual three dimensional structure or, alternatively, to an essentially random three dimensional structure, i.e., totally denatured. The invention encompasses reagents of, and methods using, polypeptide variants, e.g., involving glycosylation, phosphorylation, sulfation, disulfide bond formation, deamidation, isomerization, cleavage points in signal or leader sequence processing, covalent and non-covalently bound cofactors, oxidized variants, and the like. The formation of disulfide linked proteins is described (see, e.g., Woycechowsky and Raines (2000) Curr. Opin. Chem. Biol. 4:533-539; Creighton, et al. (1995) Trends Biotechnol. 13:18-23).

“Recombinant” when used with reference, e.g., to a nucleic acid, cell, animal, virus, plasmid, vector, or the like, indicates modification by the introduction of an exogenous, non-native nucleic acid, alteration of a native nucleic acid, or by derivation in whole or in part from a recombinant nucleic acid, cell, virus, plasmid, or vector. Recombinant protein refers to a protein derived, e.g., from a recombinant nucleic acid, virus, plasmid, vector, or the like. “Recombinant bacterium” encompasses a bacterium where the genome is engineered by recombinant methods, e.g., by way of a mutation, deletion, insertion, and/or a rearrangement. “Recombinant bacterium” also encompasses a bacterium modified to include a recombinant extra-genomic nucleic acid, e.g., a plasmid or a second chromosome, or a bacterium where an existing extra-genomic nucleic acid is altered.

“Sample” refers to a sample from a human, animal, placebo, or research sample, e.g., a cell, tissue, organ, fluid, gas, aerosol, slurry, colloid, or coagulated material. The “sample” may be tested in vivo, e.g., without removal from the human or animal, or it may be tested in vitro. The sample may be tested after processing, e.g., by histological methods. “Sample” also refers, e.g., to a cell comprising a fluid or tissue sample or a cell separated from a fluid or tissue sample. “Sample” may also refer to a cell, tissue, organ, or fluid that is freshly taken from a human or animal, or to a cell, tissue, organ, or fluid that is processed or stored.

A “selectable marker” encompasses a nucleic acid that allows one to select for or against a cell that contains the selectable marker. Examples of selectable markers include, without limitation, e.g.: (1) A nucleic acid encoding a product providing resistance to an otherwise toxic compound (e.g., an antibiotic), or encoding susceptibility to an otherwise harmless compound (e.g., sucrose); (2) A nucleic acid encoding a product that is otherwise lacking in the recipient cell (e.g., tRNA genes, auxotrophic markers); (3) A nucleic acid encoding a product that suppresses an activity of a gene product; (4) A nucleic acid that encodes a product that can be readily identified (e.g., phenotypic markers such as beta-galactosidase, green fluorescent protein (GFP), cell surface proteins, an epitope tag, a FLAG tag); (5) A nucleic acid that can be identified by hybridization techniques, for example, PCR or molecular beacons.

“Specifically” or “selectively” binds, when referring to a ligand/receptor, nucleic acid/complementary nucleic acid, antibody/antigen, or other binding pair (e.g., a cytokine to a cytokine receptor) indicates a binding reaction which is determinative of the presence of the protein in a heterogeneous population of proteins and other biologics. Thus, under designated conditions, a specified ligand binds to a particular receptor and does not bind in a significant amount to other proteins present in the sample. Specific binding can also mean, e.g., that the binding compound, nucleic acid ligand, antibody, or binding composition derived from the antigen-binding site of an antibody, of the contemplated method binds to its target with an affinity that is often at least 25% greater, more often at least 50% greater, most often at least 100% (2-fold) greater, normally at least ten times greater, more normally at least 20-times greater, and most normally at least 100-times greater than the affinity with any other binding compound.

In a typical embodiment an antibody will have an affinity that is greater than about 10⁹ liters/mol, as determined, e.g., by Scatchard analysis (Munsen, et al. (1980) Analyt. Biochem. 107:220-239). It is recognized by the skilled artisan that some binding compounds can specifically bind to more than one target, e.g., an antibody specifically binds to its antigen, to lectins by way of the antibody's oligosaccharide, and/or to an Fc receptor by way of the antibody's Fc region.

“Spread” of a bacterium encompasses “cell to cell spread,” that is, transmission of the bacterium from a first host cell to a second host cell, as mediated, for example, by a vesicle. Functions relating to spread include, but are not limited to, e.g., formation of an actin tail, formation of a pseudopod-like extension, and formation of a double-membraned vacuole.

The term “subject” as used herein refers to a human or non-human organism. Thus, the methods and compositions described herein are applicable to both human and veterinary disease. In certain embodiments, subjects are “patients,” i.e., living humans that are receiving medical care for a disease or condition. This includes persons with no defined illness who are being investigated for signs of pathology. Preferred are subjects who have an existing plasmodium infection.

The “target site” of a recombinase is the nucleic acid sequence or region that is recognized, bound, and/or acted upon by the recombinase (see, e.g., U.S. Pat. No. 6,379,943 issued to Graham, et al.; Smith and Thorpe (2002) Mol. Microbiol. 44:299-307; Groth and Calos (2004) J. Mol. Biol. 335:667-678; Nunes-Duby, et al. (1998) Nucleic Acids Res. 26:391-406).

“Therapeutically effective amount” is defined as an amount of a reagent or pharmaceutical composition that is sufficient to show a patient benefit, i.e., to cause a decrease, prevention, or amelioration of the symptoms of the condition being treated. When the agent or pharmaceutical composition comprises a diagnostic agent, a “diagnostically effective amount” is defined as an amount that is sufficient to produce a signal, image, or other diagnostic parameter. Effective amounts of the pharmaceutical formulation will vary according to factors such as the degree of susceptibility of the individual, the age, gender, and weight of the individual, and idiosyncratic responses of the individual (see, e.g., U.S. Pat. No. 5,888,530 issued to Netti, et al.).

“Treatment” or “treating” (with respect to a condition or a disease) is an approach for obtaining beneficial or desired results including and preferably clinical results. For purposes of this invention, beneficial or desired results with respect to a disease include, but are not limited to, one or more of the following: improving a condition associated with a disease, curing a disease, lessening severity of a disease, delaying progression of a disease, alleviating one or more symptoms associated with a disease, increasing the quality of life of one suffering from a disease, and/or prolonging survival Likewise, for purposes of this invention, beneficial or desired results with respect to a condition include, but are not limited to, one or more of the following: improving a condition, curing a condition, lessening severity of a condition, delaying progression of a condition, alleviating one or more symptoms associated with a condition, increasing the quality of life of one suffering from a condition, and/or prolonging survival.

“Vaccine” encompasses preventative vaccines. Vaccine also encompasses therapeutic vaccines, e.g., a vaccine administered to a mammal that comprises a condition or disorder associated with the antigen or epitope provided by the vaccine.

2. Plasmodium Antigens

While the following examples address the use of Plasmodium falciparum antigen sequences, this is exemplary in nature only, and other Plasmodium species may find use in the methods and compositions described herein.

As used herein, the term “wild-type Plasmodium antigen” refers to a polypeptide encoding an amino acid sequence which comprises a sequence obtainable from a natural, as opposed to a recombinant, source. The following sequences serve to distinguish between exemplary wild-type sequences, and derived sequences finding use in the present invention, examples of which are described herein:

Wild type P. falciparum CelTOS sequence (182 aa):
>qi|124805898|ref|XP_001350569.1| CelTOS, putative [Plasmodium falciparum 3D7]
(SEQ ID NO: 18)
MNALRRLPVICSFLVFLVFSNVLCFRGNNGHNSSSSLYNGSQFIEQLNNSFTSAFLESQS
MNKIGDDLAETISNELVSVLQKNSPTFLESSFDIKSEVKKHAKSMLKELIKVGLPSFENL
VAENVKPPKVDPATYGIIVPVLISLFNKVETAVGAKVSDEIWNYNSPDVSESEESLSDDF
FD
Derivative codon optimized for Lm expression (aa 25-182 of WT sequence):
(SEQ ID NO: 19)
FRGNNGHNSSSSLYNGSQFIEQLNNSFTSAFLESQSMNKIGDDLAETISNELVSVLQKNS
PTFLESSFDIKSEVKKHAKSMLKELIKVGLPSFENLVAENVKPPKVDPATYGIIVPVLTS
LFNKVETAVGAKVSDEIWNYNSPDVSESEESLSDDFFD
Celtos sequence for vaccine strains (1-158 of synthetic sequence):
(SEQ ID NO: 11)
FRGNNGHNSSSSLYNGSQFIEQLNNSFTSAFLESQSMNKIGDDLAETISNELVSVLQKNS
PTFLESSFDIKSEVKKHAKSMLKELIKVGLPSFENLVAENVKPPKVDPATYGIIVPVLTS
LFNKVETAVGAKVSDEIWNYNSPDVSESEESLSDDFFD
Wild type P. falciparum CSP sequence (397 aa):
>gi|124504759|ref|XP_001351122.1| circumsporozoite (CS) protein
[Plasmodium falciparum 3D7]
(SEQ ID NO: 20)
MMRKLAILSVSSFLFVEALFQEYQCYGSSSNTRVLNELNYDNAGINLYNELEMNYYGKQE
NWYSLKKNSRSLGENDDGNNEDNEKLRKPKHKKLKQPADGNPDPNANPNVDPNANPNVDP
NANPNVDPNANPNANPNANPNANPNANPNANPNANPNANPNANPNANPNANPNANPNANP
NANPNANPNANPNANPNVDPNANPNANPNANPNANPNANPNANPNANPNANPNANPNANP
NANPNANPNANPNANPNANPNANPNANPNANPNKNNQGNGQGHNMPNDPNRNVDENANAN
SAVKNNNNEEPSDKHIKEYLNKIQNSLSTEWSPCSVTCGNGIQVRIKPGSANKPKDELDY
ANDIEKKICKMEKCSSVFNVVNSSIGLIMVLSFLFLN
Derivative codon optimized for Lm expression (aa 21-140,
minimized repeat sequence, 273-397 of WT sequence) (235 aa total):
(SEQ ID NO: 21)
QEYQCYGSSSNTRVLNELNYDNAGTNLYNELEMNYYGKQENWYSLKKNSRSLGENDDGNN
EDNEKLRKPKHKKLKQPADGNPDPNANPNVDPNANPNVNANPNANPNANPNKNNQGNGQG
HNMPNDPNRNVDENANANSAVKNNNNEEPSDKHIKEYLNKIQNSLSTEWSPCSVICGNGI
QVRIKPGSANKPKDELDYANDIEKKICKMEKCSSVFNVVNSSIGLIMVLSFLFLN
CSP sequence for Lm vaccine strains (1-224 of synthetic sequence):
(SEQ ID NO: 9)
QEYQCYGSSSNTRVLNELNYDNAGTNLYNELEMNYYGKQENWYSLKKNSRSLGENDDGNN
EDNEKLRKPKHKKLKQPADGNPDPNANPNVDPNANPNVNANPNANPNANPNKNNQGNGQG
HNMPNDPNRNVDENANANSAVKNNNNEEPSDKHIKEYLNKIQNSLSTEWSPCSVICGNGI
QVRIKPGSANKPKDELDYANDIEKKICKMEKCSSVFNVVNSSIG
Wild type P. falciparum LSA1 sequence (1909 aa):
>gi|9916|emb|CAA39663.1| liver stage antigen [Plasmodium falciparum]
(SEQ ID NO: 22)
NKHILYISFYFILVNLLIFHINGKIIKNSEKDEIIKSNLRSGSSNSRNRINEEKHEKKHVSLHNSYEKTK
NNENNKFFDKDKELTMSNVKNVSQTNFKSLLRNLGVSENIFLKENKLNKEGKLIEHIINDDDDKKKYIKG
QDENRQEDLEEKAAKETLQGQQSDLEQERLAKEKLQEQQSDSEQERLAKEKLQEQQSDLEQERLAKEKLQ
EQQSDLEQERLAKEKLQEQQSDLEQERRAKEKLQEQQSDLEQERRAKEKLQEQQSDLEQERRAKEKLQEQ
QSDLEQERLAKEKLQEQQSDLEQERRAKEKLQEQQSDLEQERLAKEKLQEQQSDLEQERLAKEKLQEQQS
DLEQERLAKEKLQGQQSDLEQERLAKEKLQEQQSDLEQDRLAKEKLQEQQSDLEQERLAKEKLQEQQSDL
EQERRAKEKLQEQQSDLEQERLAKEKLQEQQSDLEQERRAKEKLQEQQSDLEQERRAKEKLQEQQSDLEQ
ERLAKEKLQEQQSDLEQERLAKEKLQEQQSDSEQERLAKEKLQEQQSDLEQERLAKEKLQEQQSDLEQER
LAKEKLQEQQSDLEQERLAKEKLQEQQSDLEQERLAKEKLQGQQSDLEQERLAKEKLQGQQSDLEQERLA
KEKLQEQQSDLEQERLAKEKLQEQQSDLERTAKSKETLQEQQSDLEQERLAKEKLQEQQSDLEQERRAKE
KLQEQQSDLEQERRAKEKLQEQQSDLEQERRAKEKLQEQQSDLEQERRAKEKLQEQQSDLEQDRLAKEKL
QEQQSDLEQERRAKEKLQEQQSDLEQDRLAKEKLQEQQSDLEQERRAKEKLQEQQSDLEQERLAKEKLQE
QQSDLEQERRAKEKLQEQQSDLEQDRLAKEKLQEQQSDLEQERRAKEKLQEQQSDLEQERRAKEKLQEQQ
SLERQERLAKEKLQEQQRDLEQERRAKEKLQEQQSDLEQERRAKEKLQEQQSDLEQERLAKEKLQEQQSD
LEQERLAKEKLQEQQSDLEQERLAKEKLQGQQSDLEQERLAKEKLQGQQSDLEQERLAKEKLQEQQSDLE
QERLAKEKLQEQQSDLEQERLAKEKLQGQQSDLEQERLAKEKLQGQQSDLEQERLAKEKLQGQQSDLEQE
RLAKEKLQGQQSDLEQERLAKEKLQEQQSDLEQERLAKEKLQEQQSDLEQERRAKEKLQEQQSDLERTKA
SKETLQEQQSDLEQERLAKEKLQEQQSDLEQERRAKEKLQEQQSDLEQERLAKEKLQEQQSDLEQERRAK
EKLQEQQSDLEQERRAKEKLQEQQSDLEQERRAKEKLQEQQSDLEQERLAKEKLQEQQSDLEQERLAKEK
LQEQQSDLEQERRAKEKLQEQQSDLEQERLAKEKLQEQQSDLEQERRAKEKLQEQQSDLEQDRLAKEKLQ
EQQRDLEQERRAKEKLQEQQSDLEQERRAKEKLQEQQSDLEQERRAKEKLQEQQSDLEQERRAKEKLQEQ
QSDLEQERLAKEKLQEQQRDLEQERRAKEKLQEQQSDLEQERRAKEKLQEQQSDLEQERLANEKLQEQQR
DLEQERRAKEKLQEQQSDLEQERRAKEKLQEQQSDLEQERRAKEKLQEQQSDLEQERLAKEKLQEQQRDL
EQERLAKEKLQEQQRDLEQRKADTKKNLERKKEHGDVLAEDLYGRLEIPAIELPSENERGYYIPHQSSLP
QDNRGNSRDSKEISIIEKTNRESITTNVEGRRDIHKGHLEEKKDGSIKPEQKEDKSADIQNHTLETVNIS
EQERLAKEKLQEQQRDLEQRKADTKKNLERKKEHGDVLAEDLYGRLEIPAIELPSENERGYYIPHQSSLP
QDNRGNSRDSKEISIIEKTNRESITTNVEGRRDIHKGHLEEKKDGSIKPEQKEDKSADIQNHTLETVNIS
DVNDFQISKYEDEISAEYDDSLIDEEEDDEDLDEFKPIVQYDNFQDEENIGIYKELEDLIEKNENLDDLD
EGIEKSSEELSEEKIKKGKKYEKTKDNNFKPNDKSLYDEHIHHYKNDKQVNKEKEKFIKSLFHIFDGDNE
ILQIVDELSEDITKYFMKL
Derivative codon optimized for Lm expression (aa 28-154,
minimized LSA1 repeat sequence, 1630-1909 of WT sequence) (475 aa total):
(SEQ ID NO: 23)
NSEKDEIIKSNLRSGSSNSRNRINEEKHEKKHVLSHNSYEKTKNNENNKFFDKDKELTMS
NVKNVSQTNFKSLLRNLGVSENIFLKENKLNKEGKLIEHIINDDDDKKKYIKGQDENRQE
DLEEKAAEQQSDLEQERLAKEKLQEQQSDLEQERLAKEKLQERLAKEKLQEQQRDLEQER
LAKEKLQEQQRDLEQRKADTKKNLERKKEHGDVLAEDLYGRLEIPAIELPSENERGYYIP
HQSSLPQDNRGNSRDSKEISIIEKTNRESITTNVEGRRDIHKGHLEEKKDGSIKPEQKED
KSADIQNHTLETNVISDVNDFQISKYEDEISAEYDDSLIDEEEDDEDLDEFKPIVQYDNF
QDEENIGIYKELEDLIEKNENLDDLDEGIEKSSEELSEEKIKKGKKYEKTKDNNFKPNDK
SLYDEHIKKYKNDKQVNKEKEKFIKSLFHIFDGDNEILQIVDELSEDITKYFMKL
LSA1 sequence for Lm vaccine strains (1-475 of synthetic sequence):
(SEQ ID NO: 13)
NSEKDEIIKSNLRSGSSNSRNRINEEKHEKKHVLSHNSYEKTKNNENNKFFDKDKELTMS
NVKNVSQTNFKSLLRNLGVSENIFLKENKLNKEGKLIEHIINDDDDKKKYIKGQDENRQE
DLEEKAAEQQSDLEQERLAKEKLQEQQSDLEQERLAKEKLQERLAKEKLQEQQRDLEQER
LAKEKLQEQQRDLEQRKADTKKNLERKKEHGDVLAEDLYGRLEIPAIELPSENERGYYIP
HQSSLPQDNRGNSRDSKEISIIEKTNRESITTNVEGRRDIHKGHLEEKKDGSIKPEQKED
KSADIQNHTLETVNISDVNDFQISKYEDEISAEYDDSLIDEEEDDEDLDEFKPIVQYDNF
QDEENIGIYKELEDLIEKNENLDDLDEGIEKSSEELSEEKIKKGKKYEKTKDNNFKPNDK
SLYDEHIKKYKNDKQVNKEKEKFIKSLFHIFDGDNEILQIVDELSEDITKYFMKL
Wild type P. falciparum TRAP sequence (559 aa):
>gi|10048261|gb|AAG12328.1|AF249739_1 sporozite surface protein 2
[Plasmodium falciparum]
(SEQ ID NO: 24)
MNHLGNVKYLVIVFLIFFDLFLVNGRDVQNNIVDEIKYREEVCNDEVDLYLLMDCSGSIR
RHNWVNHAVPLAMKLIQQLNLNESAIHLYVNIFSNNAKEIIRLHSDASKNKEKALIIIRS
LLSTNLPYGRTNLSDALLQVRKHLNDRINRENANQLVVILTDGIPDSIQDSLKESRKLND
RGVKIAVFGIGQGINVAFNRFLVGCHPSDGKCNLYADSAWENVKNVIGPFMKAVCVEVEK
TASCGVWDEWSPCSVICGKGIRSRKREILHEGCTSELQEQCEEERCPPKREPLDVPDEPE
DDQPRPRGDNFAVEKPEENIIDNNPQEPSPNPEEGKGENPNGFDLDENPENPPNPDIPQQ
EPNIPEDSEKEVPSDVPKNPEDDREENFDIPKKPENKHDNQNNLPNDKSDRSIPYSPLPP
KVLDNERKQSDPQSQDNNGNRHVPNSEDRETRPHGRNNENRSYNRKYNDTPKHPEREEHE
KPDNNKKKGGSDNKYKIAGGIAGGLALLACAGLAYKFVVPGAATPYAGEPAPFDETLGEE
DKDLDEPEQFRLPEENEWN
Derivative codon optimized for Lm expression (aa 24-559) (536 aa total):
(SEQ ID NO: 25)
NGRDVQNNIVDEIKYREEVCNDEVDLYLLMDCSGSIRRHNWVNHAVPLAMKLIQQLNLNE
SAIHLYVNIFSNNAKEIIRLHSDASKNKEKALIIIRSLLSTNLPYGRTNLSDALLQVRKH
LNDRINRENANQLVVILIDGIPDSIQDSLKESRKLNDRGVKIAVFGIGQGINVAFNRFLV
GCHPSDGKCNLYADSAWENVKNVIGPFMKAVCVEVEKTASCGVWDEWSPCSVICGKGIRS
RKREILHEGCTSELQEQCEEERCPPKREPLDVPDEPEDDQPRPRGDNFAVEKPEENIIDN
NPQEPSPNPEEGKGENPNGFDLDENPENPPNPDIPQQEPNIPEDSEKEVPSDVPKNPEDD
REENFDIPKKPENKHDNQNNLPNDKSDRSIPYSPLPPKVLDNERKQSDPQSQDNNGNRHV
PNSEDRETRPHGRNNENRSYNRKYNDTPKHPEREEHEKPDNNKKKGGSDNKYKIAGGIAG
GLALLACAGLAYKFVVPGAATPYAGEPAPFDETLGEEDKDLDEPEQFRLPEENEWN
TRAP sequence for Lm vaccine strains (1-474 of synthetic sequence):
(SEQ ID NO: 17)
NGRDVQNNIVDEIKYREEVCNDEVDLYLLMDCSGSIRRHNWVNHAVPLAMKLIQQLNLNE
SAIHLYVNIFSNNAKEIIRLHSDASKNKEKALIIIRSLLSTNLPYGRTNLSDALLQVRKH
LNDRINRENANQLVVILIDGIPDSIQDSLKESRKLNDRGVKIAVFGIGQGINVAFNRFLV
GCHPSDGKCNLYADSAWENVKNVIGPFMKAVCVEVEKTASCGVWDEWSPCSVICGKGIRS
RKREILHEGCTSELQEQCEEERCPPKREPLDVPDEPEDDQPRPRGDNFAVEKPEENIIDN
NPQEPSPNPEEGKGENPNGFDLDENPENPPNPDIPQQEPNIPEDSEKEVPSDVPKNPEDD
REENFDIPKKPENKHDNQNNLPNDKSDRSIPYSPLPPKVLDNERKQSDPQSQDNNGNRHV
PNSEDRETRPHGRNNENRSYNRKYNDTPKHPEREEHEKPDNNKKKGGSDNKYKI

As noted, the antigen(s) used in the present invention may comprise sequences “derived from” one or more such wild-type sequences. By “derived from” as used herein is meant a polypeptide comprising one or more isolated epitopes from a specified wild-type polypeptide, or a peptide or polypeptide that is immunologically cross reactive with a specified wild-type polypeptide. In some embodiments, an antigen that is “derived from” a wild-type polypeptide comprises a partial sequence (“a fragment”) of the wild-type polypeptide. Thus, an “derived antigen” can refer to a polypeptide encoding an amino acid sequence comprising at least 8 amino acids, at least 12 amino acids, at least 20 amino acids, at least 30 amino acids, at least 50 amino acids, at least 75 amino acids, at least 100 amino acids, or at least 200 amino acids or more, obtained from a wild-type polypeptide.

The antigen can comprise a sequence encoding at least one MHC class I epitope and/or at least one MHC class II epitope obtained from an original (full-length) Plasmodium antigen. Publicly available algorithms can be used to select epitopes that bind to MHC class I and/or class II molecules. For example, the predictive algorithm “BIMAS” ranks potential HLA binding epitopes according to the predictive half-time disassociation of peptide/HLA complexes. The “SYFPEITHI” algorithm ranks peptides according to a score that accounts for the presence of primary and secondary HLA-binding anchor residues. Both computerized algorithms score candidate epitopes based on amino acid sequences within a given protein that have similar binding motifs to previously published HLA binding epitopes. Other algorithms can also be used to identify candidates for further biological testing.

The derivative of an antigen may also comprise an amino acid sequence which has at least about 80% sequence identity, at least about 85% sequence identity, at least about 90% sequence identity, at least about 95% sequence identity, or at least about 98% sequence identity to the portion of the wild-type polypeptide from which it is derived.

By “immunogenic” as that term is used herein is meant that the antigen is capable of eliciting an antigen-specific humoral or T-cell response (CD4+ and/or CD8+). Selection of one or more antigens or derivatives thereof for use in the vaccine compositions of the present invention may be performed in a variety of ways, including an assessment of the ability of a bacterium of choice to successfully express and secrete the recombinant antigen(s); and/or the ability of the recombinant antigen(s) to initiate an antigen specific CD4+ and/or CD8+ T cell response. As discussed hereinafter, in order to arrive at a final selection of antigen(s) for use with a particular bacterial delivery vehicle, these attributes of the recombinant antigen(s) are preferably combined with the ability of the complete vaccine platform (meaning the selected bacterial expression system for the selected antigen(s)) to initiate both the innate immune response as well as an antigen-specific T cell response against the recombinantly expressed antige(s). An initial determination of suitable antigens may be made by selecting antigen(s) or antigen fragment(s) that are successfully recombinantly expressed by the bacterial host of choice (e.g., Listeria), and that are immunogenic.

In certain embodiments, the antigens of the present invention are derived from a wild-type Plasmodium sequence by deleting at least one region of hydrophobicity that is 50% or greater compared to the peak hydrophobicity of Listeria ActA protein or a fragment thereof used as part of a fusion construct to express the antigen(s) of interest. Preferably, antigens are modified to have no region of hydrophobicity that exceeds 70% of the peak hydrophobicity of Listeria ActA-N100, more preferably, antigens are modified to have no region of hydrophobicity that exceeds 80% of the the peak hydrophobicity of Listeria ActA-N100; still more preferably, antigens are modified to have no region of hydrophobicity that exceeds 90% of the peak hydrophobicity of Listeria ActA-N100, and in certain embodiments, antigens are modified to have no region of hydrophobicity that exceeds the peak hydrophobicity of Listeria ActA-N100, in each case measured by the method of Kyte and Doolittle: “A Simple Method for Displaying the Hydropathic Character of a Protein”. J. Mol. Biol. 157(1982)105-132.

Direct detetection of expression of the recombinant antigen in the Western blot may be performed using an antibody that detects a Plasmodium-derived antigen sequence being recombinantly produced, or using an antibody that detects a non-Plasmodium-derived sequence (a “tag”) which is expressed with the Plasmodium-derived antigen as a fusion protein. In examples described hereinafter, the antigen(s) are expressed as fusions with an N-terminal portion of the Listeria ActA protein, and an anti-ActA antibody raised against a synthetic peptide (ATDSEDSSLNTDEWEEEK (SEQ ID NO:24)) corresponding to the mature N terminal 18 amino acids of ActA can be used to detect the expressed protein product.

Assays for testing the immunogenicity of antigens are described herein and are well known in the art. As an example, an antigen recombinantly produced by a bacterium of choice can be optionally constructed to contain the nucleotide sequence encoding an eight amino SIINFEKL (SEQ ID NO:25) peptide (also known as SL8 and ovalbumin₂₅₇₋₂₆₄), positioned in-frame at the carboxyl terminus of the antigen. Compositions such as the C-terminal SL8 epitope serve as a surrogate (i) to demonstrate that the recombinant antigen is being expressed in its entirety from N-terminal to C-terminal, and (ii) to demonstrate the ability of antigen presenting cells to present the recombinant antigen via the MHC class I pathway, using an in vitro antigen presentation assay. Such a presentation assay can be performed using the cloned C57BL/6-derived dendritic cell line DC2.4 together with the B3Z T cell hybridoma cell line as described hereinafter.

Alternatively, or in addition, immunogenicity may be tested using an ELISPOT assay as described hereinafter. ELISPOT assays were originally developed to enumerate B cells secreting antigen-specific antibodies, but have subsequently been adapted for various tasks, especially the identification and enumeration of cytokine-producing cells at the single cell level. Spleens may be harvested from animals inoculated with an appropriate bacterial vaccine, and the isolated splenocytes incubated overnight with or without peptides derived from the one or more Plasmodium antigens expressed by the bacterial vaccine. An immobilized antibody captures any secreted IFN-γ, thus permitting subsequent measurement of secreted IFN-γ, and assessment of the immune response to the vaccine.

3. Bacterial Expression Systems—the “Vaccine Platform”

Selection of a vaccine platform for delivery of the Plasmodium-derived antigens is another critical component for an effective vaccine. A number of bacterial species have been developed for use as vaccines and can be used in the present invention, including, but not limited to, Shigella flexneri, Escherichia coli, Listeria monocytogenes, Yersinia enterocolitica, Salmonella typhimurium, Salmonella typhi or mycobacterium species. This list is not meant to be limiting. See, e.g., WO04/006837; WO07/103225; and WO07/117371, each of which is hereby incorporated by reference in its entirety, including all tables, figures, and claims. The bacterial vector used in the vaccine composition may be a facultative, intracellular bacterial vector. The bacterium may be used to deliver a polypeptide described herein to antigen-presenting cells in the host organism. As described herein, L. monocytogenes provides a preferred vaccine platform for expression of the Plasmodium-derived antigen(s).

Both attenuated and commensal microorganisms have been successfully used as carriers for vaccine antigens, but bacterial carriers for the Plasmodium-derived antigens or derivatives thereof are optionally attenuated or killed but metabolically active (KBMA). The genetic background of the carrier strain used in the formulation, the type of mutation selected to achieve attenuation, and the intrinsic properties of the immunogen can be adjusted to optimize the extent and quality of the immune response elicited. The general factors to be considered to optimize the immune response stimulated by the bacterial carrier include: selection of the carrier; the specific background strain, the attenuating mutation and the level of attenuation; the stabilization of the attenuated phenotype and the establishment of the optimal dosage. Other antigen-related factors to consider include: intrinsic properties of the antigen; the expression system, antigen-display form and stabilization of the recombinant phenotype; co-expression of modulating molecules and vaccination schedules.

A preferred feature of the vaccine platform is the ability to initiate both the innate immune response as well as an antigen-specific T cell response against the recombinantly expressed Plasmodium-derived antigen(s). For example, L. monocytogenes expressing the Plasmodium-derived antigen(s) described herein induce intrahepatic Type 1 interferon (IFN-α/β) and a downstream cascade of chemokines and cytokines. In response to this intrahepatic immune stimulation, NK cells and antigen presenting cells (APCs) are recruited to the liver. These cells are activated to initiate a T cell response to eradicate Lm; simultaneously a T cell response against the Plasmodium-derived antigens expressed by the L. monocytogenes vaccine platform is also mounted. In certain embodiments, the vaccine platform of the present invention induces an increase at 24 hours following delivery of the vaccine platform to the subject in the serum concentration of one or more, and preferably all, cytokines and chemokines selected from the group consisting of IL-12p70, IFN-γ, IL-6, TNF α, and MCP-1; and induces a CD4+ and/or CD8+ antigen-specific T cell response against one or more Plasmodium-derived antigens expressed by the vaccine platform. In other embodiments, the vaccine platform of the present invention also induces the maturation of resident immature liver NK cells as demonstrated by the upregulation of activation markers such as DX5, CD11b, and CD43 in a mouse model system, or by NK cell-mediated cytolytic activity measured using ⁵¹Cr-labeled YAC-1 cells that were used as target cells.

In various embodiments, the vaccines and immunogenic compositions of the present invention can comprise Listeria monocytogenes configured to express the desired Plasmodium-derived antigen(s). The ability of L. monocytogenes to serve as a vaccine vector has been reviewed in Wesikirch, et al., Immunol. Rev. 158:159-169 (1997). A number of desirable features of the natural biology of L. monocytogenes make it an attractive platform for application to a malarial vaccine. The central rationale is that the intracellular lifecycle of L. monocytogenes enables effective stimulation of CD4+ and CD8+ T cell immunity, known to be deficient in malarial infection. Multiple pathogen associated molecular pattern (PAMP) receptors including TLRs (TLR2, TLR5, TLR9) and nucleotide-binding oligomerization domains (NOD) are triggered in response to interaction with L. monocytogenes macromolecules upon infection, resulting in the panactivation of innate immune effectors and release of Th-1 polarizing cytokines, exerting a profound impact on the development of a CD4+ and CD8+ T cell response against the Plasmodium-derived antigens.

Strains of L. monocytogenes have recently been developed as effective intracellular delivery vehicles of heterologous proteins providing delivery of antigens to the immune system to induce an immune response to clinical conditions that do not permit injection of the disease-causing agent, such as cancer and HIV. See, e.g., U.S. Pat. No. 6,051,237; Gunn et al., J. Immunol., 167:6471-6479 (2001); Liau, et al., Cancer Research, 62: 2287-2293 (2002); U.S. Pat. No. 6,099,848; WO 99/25376; WO 96/14087; and U.S. Pat. No. 5,830,702), each of which is hereby incorporated by reference in its entirety, including all tables, figures, and claims. A recombinant L. monocytogenes vaccine expressing an lymphocytic choriomeningitis virus (LCMV) antigen has also been shown to induce protective cell-mediated immunity to the antigen (Shen et al., Proc. Natl. Acad. Sci. USA, 92: 3987-3991 (1995).

Attenuated and killed but metabolically active forms of L. monocytogenes useful in immunogenic compositions have been produced. WO07/103225; and WO07/117371), each of which is hereby incorporated by reference in its entirety, including all tables, figures, and claims. The ActA protein of L. monocytogenes is sufficient to promote the actin recruitment and polymerization events responsible for intracellular movement. A human safety study has reported that oral administration of an actA/plcB-deleted attenuated form of L. monocytogenes caused no serious sequelae in adults (Angelakopoulos et al., Infection and Immunity, 70:3592-3601 (2002)). Other types of attenuated forms of L. monocytogenes have also been described (see, for example, WO 99/25376 and U.S. Pat. No. 6,099,848, which describe auxotrophic, attenuated strains of Listeria that express heterologous antigens).

In certain embodiments, the L. monocytogenes used in the vaccine compositions of the present invention is a live-attenuated strain which comprises an attenuating mutation in actA and/or inlB, and preferably a deletion of all or a portion of actA and inlB (referred to herein as “Lm ΔactA/ΔinlB”), and contains recombinant DNA encoding for the expression of the Plasmodium-derived antigen(s) of interest. These antigen(s) most preferably comprise one or more immunogenic sequences obtained or derived from one or both of the NS5B NS3 consensus sequence antigens. The Plasmodium-derived antigen(s) are preferably under the control of bacterial expression sequences and are stably integrated into the L. monocytogenes genome. Such a L. monocytogenes vaccine strain therefore employs no eukaryotic transcriptional or translational elements.

The invention also contemplates a Listeria attenuated in at least one regulatory factor, e.g., a promoter or a transcription factor. The following concerns promoters. ActA expression is regulated by two different promoters (Vazwuez-Boland, et al. (1992) Infect. Immun. 60:219-230). Together, InlA and InlB expression is regulated by five promoters (Lingnau, et al. (1995) Infect. Immun. 63:3896-3903). The transcription factor prfA is required for transcription of a number of L. monocytogenes genes, e.g., hly, plcA, ActA, mpl, prfA, and iap. PrfA's regulatory properties are mediated by, e.g., the PrfA-dependent promoter (PinlC) and the PrfA-box. The present invention, in certain embodiments, provides a nucleic acid encoding inactivated, mutated, or deleted in at least one of ActA promoter, inlB promoter, PrfA, PinlC, PrfA box, and the like (see, e.g., Lalic Mullthaler, et al. (2001) Mol. Microbiol. 42:111-120; Shetron-Rama, et al. (2003) Mol. Microbiol. 48:1537-1551; Luo, et al. (2004) Mol. Microbiol. 52:39-52). PrfA can be made constitutively active by a Gly145Ser mutation, Gly155Ser mutation, or Glu77Lys mutation (see, e.g., Mueller and Freitag (2005) Infect. Immun. 73:1917-1926; Wong and Freitag (2004) J. Bacteriol. 186:6265-6276; Ripio, et al. (1997) J. Bacteriol. 179:1533-1540).

Attenuation can be effected by, e.g., heat-treatment or chemical modification. Attenuation can also be effected by genetic modification of a nucleic acid that modulates, e.g., metabolism, extracellular growth, or intracellular growth, genetic modification of a nucleic acid encoding a virulence factor, such as listerial prfA, actA, listeriolysin (LLO), an adhesion mediating factor (e.g., an internalin such as inlA or inlB), mpl, phosphatidylcholine phospholipase C (PC-PLC), phosphatidylinositol-specific phospholipase C (PI PLC; plcA gene), any combination of the above, and the like. Attenuation can be assessed by comparing a biological function of an attenuated Listeria with the corresponding biological function shown by an appropriate parent Listeria.

The present invention, in other embodiments, provides a Listeria that is attenuated by treating with a nucleic acid targeting agent, such as a cross linking agent, a psoralen, a nitrogen mustard, cis platin, a bulky adduct, ultraviolet light, gamma irradiation, any combination thereof, and the like. Typically, the lesion produced by one molecule of cross linking agent involves cross linking of both strands of the double helix. The Listeria of the invention can also be attenuated by mutating at least one nucleic acid repair gene, e.g., uvrA, uvrB, uvrAB, uvrC, uvrD, uvrAB, phrA, and/or a gene mediating recombinational repair, e.g., recA. Moreover, the invention provides a Listeria attenuated by both a nucleic acid targeting agent and by mutating a nucleic acid repair gene. Additionally, the invention encompasses treating with a light sensitive nucleic acid targeting agent, such as a psoralen, and/or a light sensitive nucleic acid cross linking agent, such as psoralen, followed by exposure to ultraviolet light.

Attenuated Listeria useful in the present invention are described in, e.g., in U.S. Pat. Publ. Nos. 2004/0228877 and 2004/0197343, each of which is incorporated by reference herein in its entirety. Various assays for assessing whether a particular strain of Listeria has the desired attenuation are provided, e.g., in U.S. Pat. Publ. Nos. 2004/0228877, 2004/0197343, and 2005/0249748, each of which is incorporated by reference herein in its entirety.

In other embodiments, the L. monocytogenes used in the vaccine compositions of the present invention is a killed but metabolically active (KBMA) platform derived from Lm ΔactA/ΔinlB, and also is deleted of both uvrA and uvrB, genes encoding the DNA repair enzymes of the nucleotide excision repair (NER) pathway, and contains recombinant DNA encoding for the expression of the Plasmodium-derived antigen(s) of interest. These antigen(s) most preferably comprise one or more immunogenic sequences obtained or derived from one or more of CSP, Ce1TOS, LSA1, and/or TRAP. The Plasmodium-derived antigen(s) are preferably under the control of bacterial expression sequences and are stably integrated into the L. monocytogenes genome. The KBMA platform is exquisitely sensitive to photochemical inactivation by the combined treatment with the synthetic psoralen, S-59, and long-wave UV light. While killed, KBMA Lm vaccines can transiently express their gene products, allowing them to escape the phagolysosome and induce functional cellular immunity and protection against wild-typeWT Lm and vaccinia virus challenge.

In certain embodiments, an attenuated or KBMA L. monocytogenes vaccine strain comprise a constitutively active prfA gene (referred to herein as PrfA* mutants). PrfA is a transcription factor activated intracellularly which induces expression of virulence genes and encoded heterologous antigens (Ags) in appropriately engineered vaccine strains. As noted above, expression of the actA gene is responsive to PrfA, and the actA promoter is a PrfA responsive regulatory element. Inclusion of a prfA G155S allele can confer significant enhanced vaccine potency of live-attenuated or KBMA vaccines. Preferred PrfA mutants are described in U.S. Provisional patent application 61/054,454, entitled COMPOSITIONS COMPRISING PRFA* MUTANT LISTERIAAND METHODS OF USE THEREOF, filed May 19, 2008, which is hereby incorporated in its entirety including all tables, figures, and claims.

The sequence of L. monocytogenes PrfA, which includes a glycine at residue 155, is as follows (SEQ ID NO: 26):

| | |
|---|---|
| MNAQAEEFKK YLETNGIKPK QFHKKELIFN QWDPQEYCIF | 50 |
| LYDGITKLTS | |
| ISENGTIMNL QYYKGAFVIM SGFIDTETSV GYYNLEVISE | 100 |
| QATAYVIKIN | |
| ELKELLSKNL THFFYVFQTL QKQVSYSLAK FNDFSINGKL | 150 |
| GSICGQLLIL | |
| TYVYGKETPD GIKITLDNLT MQELGYSSGI AHSSAVSRII | 200 |
| SKLKQEKVIV | |
| YKNSCFYVQN LDYLKRYAPK LDEWFYLACP ATWGKLN | 237 |

The sequence of L. monocytogenes PrfA*, which includes a serine at residue 155, is as follows (SEQ ID NO: 27):

| | |
|---|---|
| MNAQAEEFKK YLETNGIKPK QFHKKELIFN QWDPQEYCIF | 50 |
| LYDGITKLTS | |
| ISENGTIMNL QYYKGAFVIM SGFIDTETSV GYYNLEVISE | 100 |
| QATAYVIKIN | |
| ELKELLSKNL THFFYVFQTL QKQVSYSLAK FNDFSINGKL | 150 |
| GSICGQLLIL | |
| TYVYGKETPD GIKITLDNLT MQELGYSSGI AHSSAVSRII | 200 |
| SKLKQEKVIV | |
| YKNSCFYVQN LDYLKRYAPK LDEWFYLACP ATWGKLN | 237 |

4. Antigenic Constructs

The antigenic construct expressed by the bacterial vaccine strain of the present invention comprises at a minimum a nucleic acid encoding a secretory sequence operable within the bacterial vaccine platform to support secretion, fused to the Plasmodium-derived antigen(s) to be expressed, wherein the resulting fusion protein is operably linked to regulatory sequences (e.g., a promoter) necessary for expression of the fusion protein by the bacterial vaccine platform. The present invention is not to be limited to polypeptide and peptide antigens that are secreted, but also embraces polypeptides and peptides that are not secreted or cannot be secreted from a Listeria or other bacterium. But preferably, the Plasmodium-derived antigen(s) are expressed in a soluble, secreted form by the bacterial vaccine strain when the strain is inoculated into a recipient.

Table 1 discloses a number of non-limiting examples of signal peptides for use in fusing with a fusion protein partner sequence such as a heterologous antigen. Signal peptides tend to contain three domains: a positively charged N-terminus (1-5 residues long); a central hydrophobic comain (7-15 residues long); and a neutral but polar C-terminal domain.

TABLE 1
Bacterial signal pathway. Signal peptides are
identified by the signal peptidase site.

| | | |
|---|---|---|
| Signal peptidase site | | |
| (cleavage site | | |
| represented by ′) | Gene | Genus/species |

SecA1 pathway

| | | |
|---|---|---|
| TEA′KD (SEQ ID NO: 28) | hly (LLO) | Listeria monocytogenes |
| VYA′DT (SEQ ID NO: 29) | Usp45 | Lactococcus lactis |
| IQA′EV (SEQ ID NO: 30) | pag (protec- | Bacillus anthracis |
| | tive antigen) | |

secA2 pathway

| | | |
|---|---|---|
| ASA′ST (SEQ ID NO: 31) | iap (invasion- | Listeria monocytogenes |
| | associated | |
| | protein) p60 | |
| VGA′EG (SEQ ID NO: 32) | NamA lmo2691 | Listeria monocytogenes |
| | (autolysin) | |
| AFA′ED (SEQ ID NO: 33) | * BA_0281 | Bacillus anthracis |
| | (NLP/P60 | |
| | Family) | |
| VQA′AE (SEQ ID NO: 34) | * atl | Staphylococcus aureus |
| | (autolysin) | |

Tat pathway

| | | |
|---|---|---|
| DKA′LT (SEQ ID NO: 35) | lmo0367 | Listeria monocytogenes |
| VGA′EG (SEQ ID NO: 36) | PhoD (alka- | Bacillus subtillis |
| | line phosphatase) | |

* Bacterial autolysins secreted by sec pathway (not determined whether secAl or secA2). Secretory sequences are encompassed by the indicated nucleic acids encoded by the Listeria EGD genome (GenBank Acc. No. NC_003210) at, e.g., nucleotides 45434-456936 (inlA); nucleotides 457021-457125 (inlB); nucleotides 1860200-1860295 (inlC); nucleotides 286219-287718 (inlE); nucleotides 205819-205893 (hly gene; LLO) (see also GenBank Acc. No. P13128); nucleotides 209470-209556 (ActA) (see also GenBank Acc. No. S20887).
The referenced nucleic acid sequences, and corresponding translated amino acid sequences, and the cited amino acid sequences, and the corresponding nucleic acid sequences associated with or cited in that reference, are incorporated by reference herein in their entirety.

In certain exemplary embodiments described hereinafter, the Plasmodium-derived sequence(s) may be expressed as a single polypeptide fused to an amino-terminal portion of the L. monocytogenes ActA protein which permits expression and secretion of a fusion protein from the bacterium within the vaccinated host. In these embodiments, the antigenic construct may be a polynucleotide comprising a promoter operably linked to a nucleic acid sequence encoding a fusion protein, wherein the fusion protein comprises (a) modified ActA and (b) one or more Plasmodium-derived epitopes to be expressed as a fusion protein following the modified ActA sequence.

By “modified ActA” is meant a contiguous portion of the L. monocytogenes ActA protein which comprises at least the ActA signal sequence, but does not comprise the entirety of the ActA sequence, or that has at least about 80% sequence identity, at least about 85% sequence identity, at least about 90% sequence identity, at least about 95% sequence identity, or at least about 98% sequence identity to such an ActA sequence. The ActA signal sequence is MGLNRFMRAMMVVFITANCITINPDIIFA (SEQ ID NO: 41). In some embodiments, the promoter is ActA promoter from WO07/103225; and WO07/117371, each of which is incorporated by reference in its entirety herein.

By way of example, the modified ActA may comprise at least the first 59 amino acids of ActA, or a sequence having at least about 80% sequence identity, at least about 85% sequence identity, at least about 90% sequence identity, at least about 95% sequence identity, or at least about 98% sequence identity to at least the first 59 amino acids of ActA. In some embodiments, the modified ActA comprises at least the first 100 amino acids of ActA, or a sequence having at least about 80% sequence identity, at least about 85% sequence identity, at least about 90% sequence identity, at least about 95% sequence identity, or at least about 98% sequence identity to the first 100 amino acids of ActA. In other words, in some embodiments, the modified ActA sequence corresponds to an N-terminal fragment of ActA (including the ActA signal sequence) that is truncated at residue 100 or thereafter.

ActA-N100 has the following sequence (SEQ ID NO:37):

| | |
|---|---|
| VGLNRFMRAM MVVFITANCI TINPDIIFAA TDSEDSSLNT | 50 |
| DEWEEEKTEE | |
| QPSEVNTGPR YETAREVSSR DIEELEKSNK VKNTNKADLI | 100 |
| AMLKAKAEKG | |

In this sequence, the first residue is depicted as a valine; the polypeptide is synthesized by Listeria with a methionine in this position. Thus, ActA-N100 may also have the following sequence (SEQ ID NO:38):

| | |
|---|---|
| MGLNRFMRAM MVVFITANCI TINPDIIFAA TDSEDSSLNT | 50 |
| DEWEEEKTEE | |
| QPSEVNTGPR YETAREVSSR DIEELEKSNK VKNTNKADLI | 100 |
| AMLKAKAEKG | |

ActA-N100 may also comprise one or more additional residues lying between the C-terminal residue of the modified ActA and the Plasmodium-derived antigen sequence. In the following sequences, ActA-N100 is extended by two residues added by inclusion of a BamH1 site:

(SEQ ID NO:39)

| | |
|---|---|
| VGLNRFMRAM MVVFITANCI TINPDIIFAA TDSEDSSLNT | 50 |
| DEWEEEKTEE | |
| QPSEVNTGPR YETAREVSSR DIEELEKSNK VKNTNKADLI | 100 |
| AMLKAKAEKG | |
| GS | |

which when synthesized with a first residue methionine has the sequence:

(SEQ ID NO: 40)

| | |
|---|---|
| MGLNRFMRAM MVVFITANCI TINPDIIFAA TDSEDSSLNT | 50 |
| DEWEEEKTEE | |
| QPSEVNTGPR YETAREVSSR DIEELEKSNK VKNTNKADLI | 100 |
| AMLKAKAEKG | |
| GS. | |

Exemplary constructs are described hereinafter and in WO07/103225, which is incorporated by reference herein. ANZ-100 (formerly known as CRS-100; BB-IND 12884 and clinicaltrials.gov identifier NCT00327652) consists of a L. monocytogenes ΔactA/ΔinlB platform without any exogenous antigen expression sequences. In the exemplary constructs described in WO07/103225, this platform has been engineered to express human Mesothelin as a fusion with ActA-N100. The mesothelin expression vaccine has been evaluated in subjects with advanced carcinoma with liver metastases using CRS-207 (BB-IND 13389 and clinicaltrials.gov identifier NCT00585845). The present invention contemplates modification of this vaccine by replacing the mesothelin sequences with Plasmodium-derived antigen sequence.

As sequences encoded by one organism are not necessarily codon optimized for optimal expression in a chosen vaccine platform bacterial strain, the present invention also provides nucleic acids that are altered by codon optimized for expressing by a bacterium such as L. monocytogenes.

In various embodiments, at least one percent of any non-optimal codons are changed to provide optimal codons, more normally at least five percent are changed, most normally at least ten percent are changed, often at least 20% are changed, more often at least 30% are changed, most often at least 40%, usually at least 50% are changed, more usually at least 60% are changed, most usually at least 70% are changed, optimally at least 80% are changed, more optimally at least 90% are changed, most optimally at least 95% are changed, and conventionally 100% of any non-optimal codons are codon-optimized for Listeria expression (Table 2).

TABLE 2
Optimized codons for expression in Listeria.

| Amino Acid | A | R | N | D | C | Q | E | G | H | I |
|---|---|---|---|---|---|---|---|---|---|---|
| Optimal | GCA | CGU | AAU | GAU | UGU | CAA | GAA | GGU | CAU | AUU |
| Listeria | | | | | | | | | | |
| codon | | | | | | | | | | |
| Amino Acid | L | K | M | F | P | S | T | W | Y | V |
| Optimal | UUA | AAA | AUG | UUU | CCA | AGU | ACA | UGG | UAU | GUU |
| Listeria | | | | | | | | | | |
| codon | | | | | | | | | | |

The invention supplies a number of listerial species and strains for making or engineering a vaccine platform of the present invention. The Listeria of the present invention is not to be limited by the species and strains disclosed in Table 3.

TABLE 3
Strains of Listeria suitable for use in the present invention,
e.g., as a vaccine or as a source of nucleic acids.

| | |
|---|---|
| L. monocytogenes 10403S wild type. | Bishop and Hinrichs (1987) J. Immunol. |
| | 139: 2005-2009; Lauer, et al. (2002) J. Bact. |
| | 184: 4177-4186. |
| L. monocytogenes DP-L4056 (phage cured). The | Lauer, et al. (2002) J. Bact. 184: 4177-4186. |
| prophage-cured 10403S strain is designated DP- | |
| L4056. | |
| L. monocytogenes DP-L4027, which is DP-L2161, | Lauer, et al. (2002) J. Bact. 184: 4177-4186; Jones |
| phage cured, deleted in hly gene. | and Portnoy (1994) Infect. Immunity 65: 5608- |
| | 5613. |
| L. monocytogenes DP-L4029, which is DP-L3078, | Lauer, et al. (2002) J. Bact. 184: 4177-4186; |
| phage cured, deleted in ActA. | Skoble, et al. (2000) J. Cell Biol. 150: 527-538. |
| L. monocytogenes DP-L4042 (delta PEST) | Brockstedt, et al. (2004) Proc. Natl. Acad. Sci. |
| | USA 101: 13832-13837; supporting information. |
| L. monocytogenes DP-L4097 (LLO-S44A). | Brockstedt, et al. (2004) Proc. Natl. Acad. Sci. |
| | USA 101: 13832-13837; supporting information. |
| L. monocytogenes DP-L4364 (delta lplA; | Brockstedt, et al. (2004) Proc. Natl. Acad. Sci. |
| lipoate protein ligase). | USA 101: 13832-13837; supporting information. |
| L. monocytogenes DP-L4405 (delta inlA). | Brockstedt, et al. (2004) Proc. Natl. Acad. Sci. |
| | USA 101: 13832-13837; supporting information. |
| L. monocytogenes DP-L4406 (delta inlB). | Brockstedt, et al. (2004) Proc. Natl. Acad. Sci. |
| | USA 101: 13832-13837; supporting information. |
| L. monocytogenes CS-L0001 (delta ActA-delta | Brockstedt, et al. (2004) Proc. Natl. Acad. Sci. |
| inlB). | USA 101: 13832-13837; supporting information. |
| L. monocytogenes CS-L0002 (delta ActA-delta | Brockstedt, et al. (2004) Proc. Natl. Acad. Sci. |
| lplA). | USA 101: 13832-13837; supporting information. |
| L. monocytogenes CS-L0003 (L461T-delta lplA). | Brockstedt, et al. (2004) Proc. Natl. Acad. Sci. |
| | USA 101: 13832-13837; supporting information. |
| L. monocytogenes DP-L4038 (delta ActA-LLO | Brockstedt, et al. (2004) Proc. Natl. Acad. Sci. |
| L461T). | USA 101: 13832-13837; supporting information. |
| L. monocytogenes DP-L4384 (S44A-LLO L461T). | Brockstedt, et al. (2004) Proc. Natl. Acad. Sci. |
| | USA 101: 13832-13837; supporting information. |
| L. monocytogenes. Mutation in lipoate protein | O'Riordan, et al. (2003) Science 302: 462-464. |
| ligase (LplA1). | |
| L. monocytogenes DP-L4017 (10403S hly (L461T) | U.S. Provisional Pat. application Ser. No. |
| point mutation in hemolysin gene. | 60/490,089 filed Jul. 24, 2003. |
| L. monocytogenes EGD. | GenBank Acc. No. AL591824. |
| L. monocytogenes EGD-e. | GenBank Acc. No. NC_003210. ATCC Acc. |
| | No. BAA-679. |
| L. monocytogenes strain EGD, complete genome, | GenBank Acc. No. AL591975 |
| segment 3/12 | |
| L. monocytogenes. | ATCC Nos. 13932; 15313; 19111-19120; 43248- |
| | 43251; 51772-51782. |
| L. monocytogenes DP-L4029 deleted in uvrAB. | U.S. Provisional Pat. application Ser. No. |
| | 60/541,515 filed Feb. 2, 2004; |
| | U.S. Provisional Pat. application Ser. No. |
| | 60/490,080 filed Jul. 24, 2003. |
| L. monocytogenes DP-L4029 deleted in uvrAB | U.S. Provisional Pat. application Ser. No. |
| treated with a psoralen. | 60/541,515 filed Feb. 2, 2004. |
| L. monocytogenes delta actA delta inlB delta | Brockstedt (2005) Nature Medicine and |
| uvrAB | KBMA patent |
| L. monocytogenes delta actA delta inlB delta | Brockstedt (2005) Nature Medicine and |
| uvrAB treated with psoralen | KBMA patent |
| L. monocytogenes delta artA delta inlB delta | Lauer et al, (2008) Infect. Immun. And |
| uvrAB prfA(G155S) | WO 2009/143085 |
| L. monocytogenes delta actA delta inlB delta | Lauer et al, (2008) Infect. Immun. And |
| uvrAB prfA(G155S) treated with psoralen | WO 2009/143085 |
| L. monocytogenes ActA-/inlB- double mutant. | Deposited with ATCC on Oct. 3, 2003. Acc. |
| | No. PTA-5562. |
| L. monocytogenes lplA mutant or hly mutant. | U.S. patent application No. 20040013690 of |
| | Portnoy, et al. |
| L. monocytogenes DAL/DAT double mutant. | U.S. patent application No. 20050048081 of |
| | Frankel and Portnoy. |
| L. monocytogenes str. 4b F2365. | GenBank Acc. No. NC_002973. |
| Listeria ivanovii | ATCC No. 49954 |
| Listeria innocua Clip11262. | GenBank Acc. No. NC_003212; AL592022. |
| Listeria innocua, a naturally occurring hemolytic | Johnson, et al. (2004) Appl. Environ. |
| strain containing the PrfA-regulated virulence gene | Microbiol. 70: 4256-4266. |
| cluster. | |
| Listeria seeligeri. | Howard, et al. (1992) Appl. Eviron. |
| | Microbiol. 58: 709-712. |
| Listeria innocua with L. monocytogenes | Johnson, et al. (2004) Appl. Environ. |
| pathogenicity island genes. | Microbiol. 70: 4256-4266. |
| Listeria innocua with L. monocytogenes internalin A | See, e.g., Lingnau, et al. (1995) Infection |
| gene, e.g., as a plasmid or as a genomic nucleic acid. | Immunity 63: 3896-3903; Gaillard, et al. (1991) |
| | Cell 65: 1127-1141). |

The present invention encompasses reagents and methods that comprise the above listerial strains, as well as these strains that are modified, e.g., by a plasmid and/or by genomic integration, to contain a nucleic acid encoding one of, or any combination of, the following genes: hly (LLO; listeriolysin); iap (p60); inlA; inlB; inlC; dal (alanine racemase); daaA (dat; D-amino acid aminotransferase); plcA; plcB; ActA; or any nucleic acid that mediates growth, spread, breakdown of a single walled vesicle, breakdown of a double walled vesicle, binding to a host cell, uptake by a host cell. The present invention is not to be limited by the particular strains disclosed above.

4. Therapeutic Compositions.

The vaccine compositions described herein can be administered to a host, either alone or in combination with a pharmaceutically acceptable excipient, in an amount sufficient to induce an appropriate immune response. The immune response can comprise, without limitation, specific immune response, non specific immune response, both specific and non specific response, innate response, primary immune response, adaptive immunity, secondary immune response, memory immune response, immune cell activation, immune cell proliferation, immune cell differentiation, and cytokine expression. The vaccines of the present invention can be stored, e.g., frozen, lyophilized, as a suspension, as a cell paste, or complexed with a solid matrix or gel matrix.

In certain embodiments, after the subject has been administered an effective dose of a vaccine containing the immunogenic Plasmodium-derived antigen polypeptides to prime the immune response, a second vaccine is administered. This is referred to in the art as a “prime-boost” regimen. In such a regimen, the compositions and methods of the present invention may be used as the “prime” delivery, as the “boost” delivery, or as both a “prime” and a “boost.”

As an example, a first vaccine comprised of killed but metabolically active Listeria that encodes and expresses the antigen polypeptide(s) may be delivered as the “prime,” and a second vaccine comprised of attenuated (live or killed but metabolically active) Listeria that encodes the antigen polypeptide(s) may be delivered as the “boost.” It should be understood, however, that each of the prime and boost need not utilize the methods and compositions of the present invention. Rather, the present invention contemplates the use of other vaccine modalities together with the bacterial vaccine methods and compositions of the present invention. The following are examples of suitable mixed prime-boost regimens: a DNA (e.g., plasmid) vaccine prime/bacterial vaccine boost; a viral vaccine prime/bacterial vaccine boost; a protein vaccine prime/bacterial vaccine boost; a DNA prime/bacterial vaccine boost plus protein vaccine boost; a bacterial vaccine prime/DNA vaccine boost; a bacterial vaccine prime/viral vaccine boost; a bacterial vaccine prime/protein vaccine boost; a bacterial vaccine prime/bacterial vaccine boost plus protein vaccine boost; etc. This list is not meant to be limiting

The prime vaccine and boost vaccine may be administered by the same route or by different routes. The term “different routes” encompasses, but is not limited to, different sites on the body, for example, a site that is oral, non-oral, enteral, parenteral, rectal, intranode (lymph node), intravenous, arterial, subcutaneous, intramuscular, intratumor, peritumor, infusion, mucosal, nasal, in the cerebrospinal space or cerebrospinal fluid, and so on, as well as by different modes, for example, oral, intravenous, and intramuscular.

An effective amount of a prime or boost vaccine may be given in one dose, but is not restricted to one dose. Thus, the administration can be two, three, four, five, six, seven, eight, nine, ten, eleven, twelve, thirteen, fourteen, fifteen, sixteen, seventeen, eighteen, nineteen, twenty, or more, administrations of the vaccine. Where there is more than one administration of a vaccine or vaccines in the present methods, the administrations can be spaced by time intervals of one minute, two minutes, three, four, five, six, seven, eight, nine, ten, or more minutes, by intervals of about one hour, two hours, three, four, five, six, seven, eight, nine, ten, 11, 12, 13, 14, 15, 16, 17, 18, 19, 20, 21, 22, 23, 24 hours, and so on. In the context of hours, the term “about” means plus or minus any time interval within 30 minutes. The administrations can also be spaced by time intervals of one day, two days, three days, four days, five days, six days, seven days, eight days, nine days, ten days, 11 days, 12 days, 13 days, 14 days, 15 days, 16 days, 17 days, 18 days, 19 days, 20 days, 21 days, and combinations thereof. The invention is not limited to dosing intervals that are spaced equally in time, but encompass doses at non-equal intervals, such as a priming schedule consisting of administration at 1 day, 4 days, 7 days, and 25 days, just to provide a non-limiting example.

In certain embodiments, administration of the boost vaccination can be initiated at about 5 days after the prime vaccination is initiated; about 10 days after the prime vaccination is initiated; about 15 days; about 20 days; about 25 days; about 30 days; about 35 days; about 40 days; about 45 days; about 50 days; about 55 days; about 60 days; about 65 days; about 70 days; about 75 days; about 80 days, about 6 months, and about 1 year after administration of the prime vaccination is initiated. Preferably one or both of the prime and boost vaccination comprises delivery of a composition of the present invention.

A “pharmaceutically acceptable excipient” or “diagnostically acceptable excipient” includes but is not limited to, sterile distilled water, saline, phosphate buffered solutions, amino acid based buffers, or bicarbonate buffered solutions. An excipient selected and the amount of excipient used will depend upon the mode of administration. Administration may be oral, intravenous, subcutaneous, dermal, intradermal, intramuscular, mucosal, parenteral, intraorgan, intralesional, intranasal, inhalation, intraocular, intramuscular, intravascular, intranodal, by scarification, rectal, intraperitoneal, or any one or combination of a variety of well-known routes of administration. The administration can comprise an injection, infusion, or a combination thereof.

Administration of the vaccine of the present invention by a non oral route can avoid tolerance. Methods are known in the art for administration intravenously, subcutaneously, intramuscularly, intraperitoneally, orally, mucosally, by way of the urinary tract, by way of a genital tract, by way of the gastrointestinal tract, or by inhalation.

An effective amount for a particular patient may vary depending on factors such as the condition being treated, the overall health of the patient, the route and dose of administration and the severity of side effects. Guidance for methods of treatment and diagnosis is available (see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK).

The vaccines of the present invention can be administered in a dose, or dosages, where each dose comprises at least 100 bacterial cells/kg body weight or more; in certain embodiments 1000 bacterial cells/kg body weight or more; normally at least 10,000 cells; more normally at least 100,000 cells; most normally at least 1 million cells; often at least 10 million cells; more often at least 100 million cells; typically at least 1 billion cells; usually at least 10 billion cells; conventionally at least 100 billion cells; and sometimes at least 1 trillion cells/kg body weight. The present invention provides the above doses where the units of bacterial administration is colony forming units (CFU), the equivalent of CFU prior to psoralen treatment, or where the units are number of bacterial cells.

The vaccines of the present invention can be administered in a dose, or dosages, where each dose comprises between 10⁷ and 10⁸ bacteria per 70 kg body weight (or per 1.7 square meters surface area; or per 1.5 kg liver weight); 2×10⁷ and 2×10⁸ bacteria per 70 kg body weight (or per 1.7 square meters surface area; or per 1.5 kg liver weight); 5×10⁷ and 5×10⁸ bacteria per 70 kg body weight (or per 1.7 square meters surface area; or per 1.5 kg liver weight); 10⁸ and 10⁹ bacteria per 70 kg body weight (or per 1.7 square meters surface area; or per 1.5 kg liver weight); between 2.0×10⁸ and 2.0×10⁹ bacteria per 70 kg (or per 1.7 square meters surface area, or per 1.5 kg liver weight); between 5.0×10⁸ to 5.0×10⁹ bacteria per 70 kg (or per 1.7 square meters surface area, or per 1.5 kg liver weight); between 10⁹ and 10¹⁰ bacteria per 70 kg (or per 1.7 square meters surface area, or per 1.5 kg liver weight); between 2×10⁹ and 2×10¹⁰ bacteria per 70 kg (or per 1.7 square meters surface area, or per 1.5 kg liver weight); between 5×10⁹ and 5×10¹⁰ bacteria per 70 kg (or per 1.7 square meters surface area, or per 1.5 kg liver weight); between 10¹¹ and 10¹² bacteria per 70 kg (or per 1.7 square meters surface area, or per 1.5 kg liver weight); between 2×10¹¹ and 2×10¹² bacteria per 70 kg (or per 1.7 square meters surface area, or per 1.5 kg liver weight); between 5×10¹¹ and 5×10¹² bacteria per 70 kg (or per 1.7 square meters surface area, or per 1.5 kg liver weight); between 10¹² and 10¹³ bacteria per 70 kg (or per 1.7 square meters surface area); between 2×10¹² and 2×10¹³ bacteria per 70 kg (or per 1.7 square meters surface area, or per 1.5 kg liver weight); between 5×10¹² and 5×10¹³ bacteria per 70 kg (or per 1.7 square meters surface area, or per 1.5 kg liver weight); between 10¹³ and 10¹⁴ bacteria per 70 kg (or per 1.7 square meters surface area, or per 1.5 kg liver weight); between 2×10¹³ and 2×10¹⁴ bacteria per 70 kg (or per 1.7 square meters surface area, or per 1.5 kg liver weight); 5×10¹³ and 5×10¹⁴ bacteria per 70 kg (or per 1.7 square meters surface area, or per 1.5 kg liver weight); between 10¹⁴ and 10¹⁵ bacteria per 70 kg (or per 1.7 square meters surface area, or per 1.5 kg liver weight); between 2×10¹⁴ and 2×10¹⁵ bacteria per 70 kg (or per 1.7 square meters surface area, or per 1.5 kg liver weight); and so on, wet weight.

Also provided is one or more of the above doses, where the dose is administered by way of one injection every day, one injection every two days, one injection every three days, one injection every four days, one injection every five days, one injection every six days, or one injection every seven days, where the injection schedule is maintained for, e.g., one day only, two days, three days, four days, five days, six days, seven days, two weeks, three weeks, four weeks, five weeks, or longer. The invention also embraces combinations of the above doses and schedules, e.g., a relatively large initial bacterialdose, followed by relatively small subsequent doses, or a relatively small initial dose followed by a large dose.

A dosing schedule of, for example, once/week, twice/week, three times/week, four times/week, five times/week, six times/week, seven times/week, once every two weeks, once every three weeks, once every four weeks, once every five weeks, and the like, is available for the invention. The dosing schedules encompass dosing for a total period of time of, for example, one week, two weeks, three weeks, four weeks, five weeks, six weeks, two months, three months, four months, five months, six months, seven months, eight months, nine months, ten months, eleven months, and twelve months.

Provided are cycles of the above dosing schedules. The cycle can be repeated about, e.g., every seven days; every 14 days; every 21 days; every 28 days; every 35 days; 42 days; every 49 days; every 56 days; every 63 days; every 70 days; and the like. An interval of non dosing can occur between a cycle, where the interval can be about, e.g., seven days; 14 days; 21 days; 28 days; 35 days; 42 days; 49 days; 56 days; 63 days; 70 days; and the like. In this context, the term “about” means plus or minus one day, plus or minus two days, plus or minus three days, plus or minus four days, plus or minus five days, plus or minus six days, or plus or minus seven days.

The present invention encompasses a method of administering Listeria that is oral. Also provided is a method of administering Listeria that is intravenous. Moreover, what is provided is a method of administering Listeria that is oral, intramuscular, intravenous, intradermal and/or subcutaneous. The invention supplies a Listeria bacterium, or culture or suspension of Listeria bacteria, prepared by growing in a medium that is meat based, or that contains polypeptides derived from a meat or animal product. Also supplied by the present invention is a Listeria bacterium, or culture or suspension of Listeria bacteria, prepared by growing in a medium that does not contain meat or animal products, prepared by growing on a medium that contains vegetable polypeptides, prepared by growing on a medium that is not based on yeast products, or prepared by growing on a medium that contains yeast polypeptides.

Methods for co-administration with an additional therapeutic agent are well known in the art (Hardman, et al. (eds.) (2001) Goodman and Gilman's The Pharmacological Basis of Therapeutics, 10th ed., McGraw-Hill, New York, N.Y.; Poole and Peterson (eds.) (2001) Pharmacotherapeutics for Advanced Practice:A Practical Approach, Lippincott, Williams & Wilkins, Phila., Pa.; Chabner and Longo (eds.) (2001) Cancer Chemotherapy and Biotherapy, Lippincott, Williams & Wilkins, Phila., Pa.).

The present invention provides reagents for administering in conjunction with a vaccine composition of the present invention. These reagents include other malarial therapeutics (including chloroquine, mefloquine, primaquine, proguanil, pyrimethamine, Fansidar (sulfadoxine-pyrimethamine)) and other immunotherapeutics. This list is not meant to be limiting. The reagents can be administered simultaneously with or independently (before or after) from the vaccine composition of the present invention. For example, the reagent can be administered immediately before (or after) the vaccine composition of the present invention, on the same day as, one day before (or after), one week before (or after), one month before (or after), or two months before (or after) the vaccine composition of the present invention, and the like.

Additional agents which are beneficial to raising a cytolytic T cell response may be used as well. Such agents are termed herein carriers. These include, without limitation, B7 costimulatory molecule, interleukin-2, interferon-γ, GM-CSF, CTLA-4 antagonists, OX-40/OX-40 ligand, CD40/CD40 ligand, sargramostim, levamisol, vaccinia virus, Bacille Calmette-Guerin (BCG), liposomes, alum, Freund's complete or incomplete adjuvant, detoxified endotoxins, mineral oils, surface active substances such as lipolecithin, pluronic polyols, polyanions, peptides, and oil or hydrocarbon emulsions. Carriers for inducing a T cell immune response which preferentially stimulate a cytolytic T cell response versus an antibody response are preferred, although those that stimulate both types of response can be used as well. In cases where the agent is a polypeptide, the polypeptide itself or a polynucleotide encoding the polypeptide can be administered. The carrier can be a cell, such as an antigen presenting cell (APC) or a dendritic cell. Antigen presenting cells include such cell types aas macrophages, dendritic cells and B cells. Other professional antigen-presenting cells include monocytes, marginal zone Kupffer cells, microglia, Langerhans' cells, interdigitating dendritic cells, follicular dendritic cells, and T cells. Facultative antigen-presenting cells can also be used. Examples of facultative antigen-presenting cells include astrocytes, follicular cells, endothelium and fibroblasts. The carrier can be a bacterial cell that is transformed to express the polypeptide or to deliver a polynucleoteide which is subsequently expressed in cells of the vaccinated individual. Adjuvants, such as aluminum hydroxide or aluminum phosphate, can be added to increase the ability of the vaccine to trigger, enhance, or prolong an immune response. Additional materials, such as cytokines, chemokines, and bacterial nucleic acid sequences, like CpG, a toll-like receptor (TLR) 9 agonist as well as additional agonists for TLR 2, TLR 4, TLR 5, TLR 7, TLR 8, TLR9, including lipoprotein, LPS, monophosphoryl lipid A, lipoteichoic acid, imiquimod, resiquimod, and other like immune modulators used separately or in combination with the described compositions are also potential adjuvants. Other representative examples of adjuvants include the synthetic adjuvant QS-21 comprising a homogeneous saponin purified from the bark of Quillaja saponaria and Corynebacterium parvum (McCune et al., Cancer, 1979; 43:1619). It will be understood that the adjuvant is subject to optimization. In other words, the skilled artisan can engage in routine experimentation to determine the best adjuvant to use.

An effective amount of a therapeutic agent is one that will decrease or ameliorate the symptoms normally by at least 10%, more normally by at least 20%, most normally by at least 30%, typically by at least 40%, more typically by at least 50%, most typically by at least 60%, often by at least 70%, more often by at least 80%, and most often by at least 90%, conventionally by at least 95%, more conventionally by at least 99%, and most conventionally by at least 99.9%.

The reagents and methods of the present invention provide a vaccine comprising only one vaccination; or comprising a first vaccination; or comprising at least one booster vaccination; at least two booster vaccinations; or at least three booster vaccinations. Guidance in parameters for booster vaccinations is available. See, e.g., Marth (1997) Biologicals 25:199-203; Ramsay, et al. (1997) Immunol. Cell Biol. 75:382-388; Gherardi, et al. (2001) Histol. Histopathol. 16:655-667; Leroux-Roels, et al. (2001) ActA Clin. Belg. 56:209-219; Greiner, et al. (2002) Cancer Res. 62:6944-6951; Smith, et al. (2003) J. Med. Virol. 70:Supp1.1:S38-541; Sepulveda-Amor, et al. (2002) Vaccine 20:2790-2795).

Formulations of therapeutic agents may be prepared for storage by mixing with physiologically acceptable carriers, excipients, or stabilizers in the form of, e.g., lyophilized powders, slurries, aqueous solutions or suspensions (see, e.g., Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.).

### EXAMPLES

The following examples serve to illustrate the present invention. These examples are in no way intended to limit the scope of the invention.

### Example 1

### Bacterial Strains and Antigen Selection

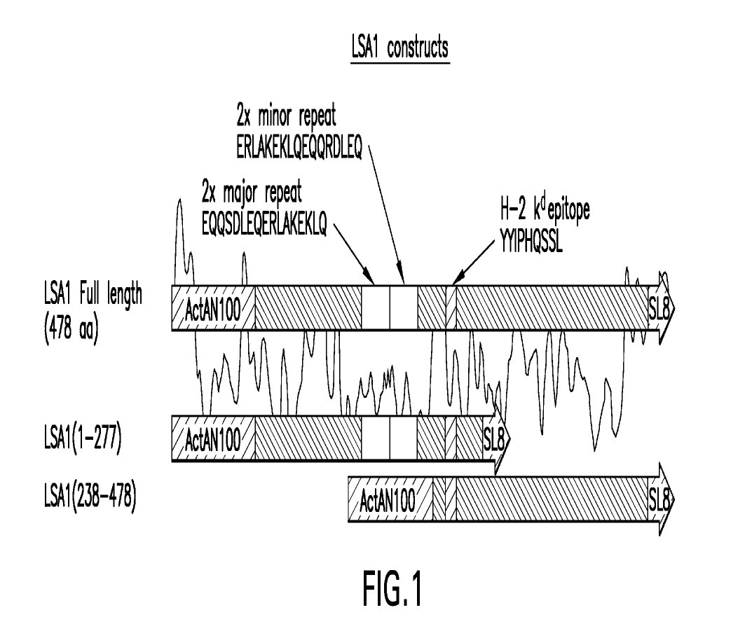

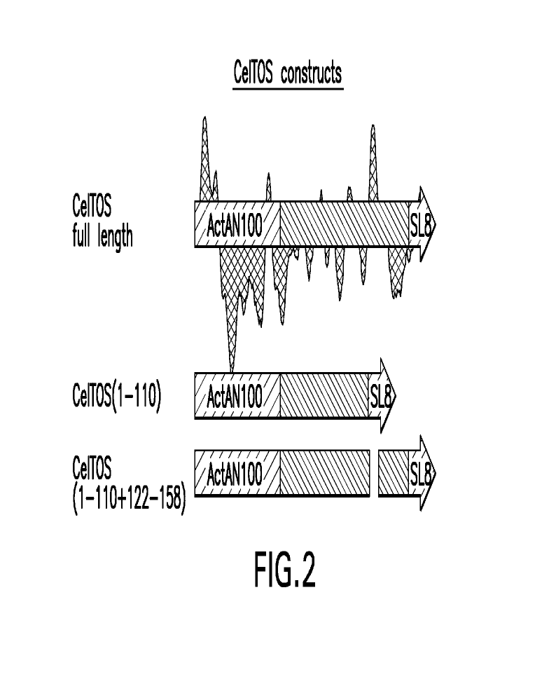

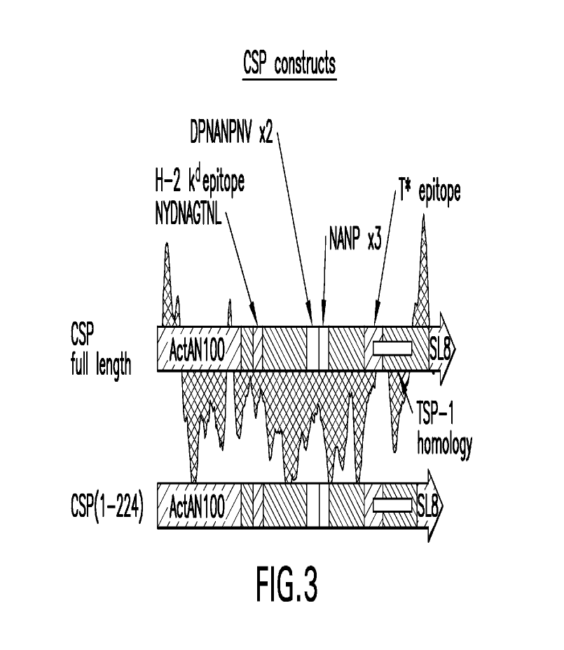

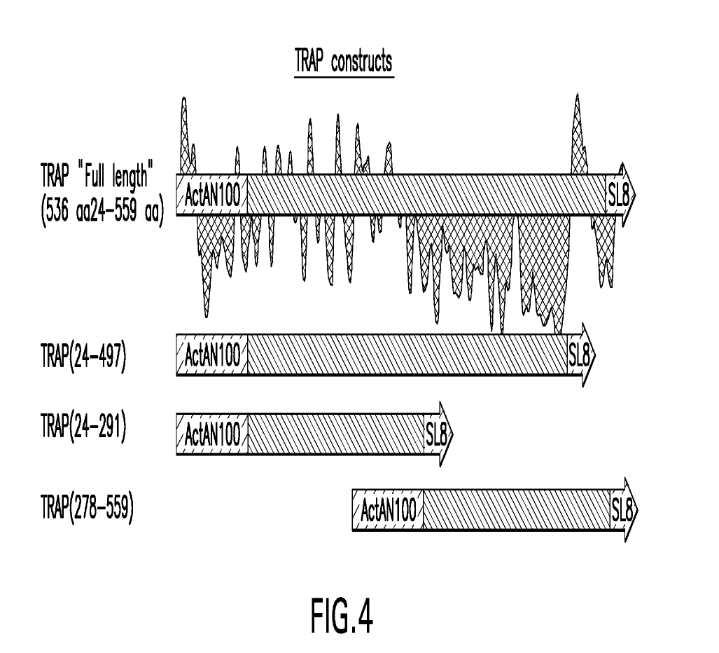

Lm vaccine strains were constructed in two strain backgrounds, live-attenuated (Lm11, aka Lm ΔactA/ΔinlB) and KBMA PrfA* (Lm677, aka Lm ΔactA/ΔinlB/ΔuvrAB/prfA G155S). Expression cassettes for the pre-erythrocytic stage P. falciparum (“Pf”) antigens CSP, LSA-1, and Ce1TOS and TRAP were analyzed for expression and secretion from Lm. The Kyte-Doolittle hydropathy plot is a widely applied scale for delineating hydrophobic character of a protein. Hydrophobicity is calculated from solvation enthalpy for an individual amino acid residue and summing the values over a sliding window of 5 to 7 amino acids. Regions with values above 0 are hydrophobic in character. An initial Kyte-Doolittle evaluation of P. falciparum antigens was used to identify regions which are less than or equal to the peak hydrophobic value obtained from ActA-N100. Values greater than this can indicate a polypeptide sequence which does not express well in Listeria. Expression cassettes were designed according to predicted hydrophobicity of antigen relative to the ActA signal sequence, and in certain constructs amino acid stretches exhibiting hydrophobicity that was 50% of the signal sequence or greater were removed (FIGS. 1-4). Malaria antigens were then synthesized with optimal codons for expression in Lm, a low G+C content organism, and repeat units in LSA-1 and Pf-CSP were minimized to conserve B and T cell epitopes, and antigen coding sequences were synthesized (DNA2.0, Menlo Park, Calif.) using optimal Listeria monocytogenes codons.

The expression cassettes were cloned as BamHI-SpeI fragments downstream from the actA promoter and in-frame with the 100 amino terminal acids of ActA (”ActA-N100″) and tagged at the carboxy terminus with SIINFEKL (SL8), a surrogate T-cell epitope that facilitates evaluation of expression and secretion of encoded heterologous antigens. The constructs were cloned into either pPL1 or a derivative of the pPL2 integration vector and stably integrated at the comK or tRNA^{Arg} locus of the bacterial chromosome respectively. CSP and Ce1TOS fusion constructs were cloned in-frame with each other (using the same strategy outlined above) by PCR that introduced new restriction sites at the 5′ (SpeI) and 3′ (MfeI) ends of the coding sequences. All molecular constructs were confirmed by DNA sequencing.

An exemplary cassette used for expression and secretion of all malaria antigens is depicted below, and contained the following domains: KpnI (ggtacc (SEQ ID NO: 1) shown below in lowercase, underlined)—actA promoter (lowercase, no underline)—ActA-N100 (uppercase, no underline)—gatccactagtcaattg (SEQ ID NO: 2) (linker sequence for in-frame cloning BamHI-SpeI-MfeI; lowercase, double underline)—SIINFEKL (SEQ ID NO: 3) T Cell tag (uppercase, underlined 87 nucleotides)—EagI (cggccg (SEQ ID NO: 4) lowercase bold):

(SEQ ID NO: 5)
ggtaccgggaagcagttggggttaactgattaacaaatgttagagaa
aaattaattctccaagtgatattcttaaaataattcatgaatatttt
ttcttatattagctaattaagaagataattaactgctaatccaattt
ttaacggaataaattagtgaaaatgaaggccgaattttccttgttct
aaaaaggttgtattagcgtatcacgaggagggagtataaGTGGGATT
AAATAGATTTATGCGTGCGATGATGGTAGTTTTCATTACTGCCAACT
GCATTACGATTAACCCCGACATAATATTTGCAGCGACAGATAGCGAA
GATTCCAGTCTAAACACAGATGAATGGGAAGAAGAAAAAACAGAAGA
GCAGCCAAGCGAGGTAAATACGGGACCAAGATACGAAACTGCACGTG
AAGTAAGTTCACGTGATATTGAGGAACTAGAAAAATCGAATAAAGTG
AAAAATACGAACAAAGCAGACCTAATAGCAATGTTGAAAGCAAAAGC
tagtcaattgGGTGACGGTAGTATTAAACTTAGC
AAAGTATTACAATTAGAAAGTATTATTAATTTTGAAAAATTAGCTGA
TGGTTCAGTTAAATAAgcggccg.

The following Plasmodium falciparum gene sequences (uppercase), optimized as discussed above, were used for expression of malarial antigens (BamH1 and SpeI restriction sites shown in lowercase at the 5′ and 3′ ends, respectively)

>Pf CSP synthetic gene (SEQ ID NO: 6):
ggatccCAAGAATATCAGTGTTATGGAAGTAGTAGCAATACTCGCGTTTTGAATGAACTA
AATTATGATAACGCAGGTACAAACTTATACAATGAATTAGAAATGAATTATTACGGTAAA
CAAGAAAATTGGTATTCGCTAAAGAAGAATAGTCGCTCATTAGGCGAGAACGATGATGGT
AATAACGAAGATAATGAGAAATTACGAAAACCTAAACATAAGAAACTTAAACAGCCGGCA
GATGGAAATCCAGACCCAAATGCAAATCCAAATGTTGATCCAAATGCGAATCCGAATGTA
AATGCTAACCCGAACGCTAATCCTAACGCAAATCCTAATAAAAATAATCAAGGAAATGGC
CAAGGACATAATATGCCAAATGATCCTAATCGTAATGTCGATGAAAATGCTAACGCTAAT
TCGGCAGTTAAAAACAATAATAACGAGGAACCAAGTGACAAACATATTAAAGAATATCTA
AACAAAATTCAAAATAGTTTATCAACGGAATGGTCGCCATGCAGTGTTACGTGTGGCAAT
GGCATACAAGTGCGCATTAAACCTGGTTCAGCGAATAAACCGAAAGACGAATTAGATTAT
GCAAATGATATTGAGAAAAAGATTTGTAAAATGGAAAAATGTAGTTCAGTCTTCAATGTA
GTGAATAGCTCAATAGGCTTAATTATGGTTCTTAGCTTCCTTTTTCTAAACactagt
Corresponding amino acid sequence
(SEQ ID NO: 7)
QEYQCYGSSSNTRVLNELNYDNAGTNLYNELEMNYYGKQENWYSLKKNSRSLGENDDGNN
EDNEKLRKPKHKKLKQPADGNPDPNANPNVDPNANPNVNANPNANPNANPNKNNQGNGQG
HNMPNDPNRNVDENANANSAVKNNNNEEPSDKHIKEYLNKIQNSLSTEWSPCSVTCGNGI
QVRIKPGSANKPKDELDYANDIEKKICKMEKCSSVFNVVNSSIGLIMVLSFLFLN
>Pf CSP(1-224)
(SEQ ID NO: 8)
ggatccCAAGAATATCAGTGTTATGGAAGTAGTAGCAATACTCGCGTTTTGAATGAACTA
AATTATGATAACGCAGGTACAAACTTATACAATGAATTAGAAATGAATTATTACGGTAAA
CAAGAAAATTGGTATTCGCTAAAGAAGAATAGTCGCTCATTAGGCGAGAACGATGATGGT
AATAACGAAGATAATGAGAAATTACGAAAACCTAAACATAAGAAACTTAAACAGCCGGCA
GATGGAAATCCAGACCCAAATGCAAATCCAAATGTTGATCCAAATGCGAATCCGAATGTA
AATGCTAACCCGAACGCTAATCCTAACGCAAATCCTAATAAAAATAATCAAGGAAATGGC
CAAGGACATAATATGCCAAATGATCCTAATCGTAATGTCGATGAAAATGCTAACGCTAAT
TCGGCAGTTAAAAACAATAATAACGAGGAACCAAGTGACAAACATATTAAAGAATATCTA
AACAAAATTCAAAATAGTTTATCAACGGAATGGTCGCCATGCAGTGTTACGTGTGGCAAT
GGCATACAAGTGCGCATTAAACCTGGTTCAGCGAATAAACCGAAAGACGAATTAGATTAT
GCAAATGATATTGAGAAAAAGATTTGTAAAATGGAAAAATGTAGTTCAGTCTTCAATGTA
GTGAATAGCTCAATAGGCactagt
Corresponding amino acid sequence
(SEQ ID NO: 9)
QEYQCYGSSSNTRVLNELNYDNAGTNLYNELEMNYYGKQENWYSLKKNSRSLGENDDGNN
EDNEKLRKPKHKKLKQPADGNPDPNANPNVDPNANPNVNANPNANPNANPNKNNQGNGQG
HNMPNDPNRNVDENANANSAVKNNNNEEPSDKHIKEYLNKIQNSLSTEWSPCSVTCGNGI
QVRIKPGSANKPKDELDYANDIEKKICKMEKCSSVFNVVNSSIG
>Pf Ce1TOS(1-158) (full length synthetic gene) (SEQ ID NO: 10):
ggatccTTCCGAGGTAATAACGGACATAATTCATCGTCTTCCTTATATAACGGGAGCCAA
TTTATAGAACAACTTAATAACAGTTTTACAAGTGCATTTTTGGAGTCACAGAGTATGAAT
AAAATCGGTGATGATCTAGCAGAAACAATCTCAAACGAATTAGTCAGTGTTCTTCAAAAA
AACTCACCAACATTTCTTGAATCGTCCTTCGACATCAAAAGTGAAGTAAAGAAACATGCG
AAAAGTATGCTTAAAGAGCTTATTAAAGTGGGCTTGCCATCGTTTGAAAACCTAGTAGCG
GAGAATGTAAAACCTCCTAAGGTCGATCCGGCGACCTATGGTATCATCGTGCCAGTTTTA
ACATCTTTGTTTAACAAAGTAGAAACTGCTGTAGGAGCTAAAGTATCGGATGAAATTTGG
AACTATAATTCGCCGGATGTTAGCGAGTCTGAAGAATCGCTAAGTGATGATTTCTTCGAC
actagt
Corresponding amino acid sequence
(SEQ ID NO: 11)
FRGNNGHNSSSSLYNGSQFIEQLNNSFTSAFLESQSMNKIGDDLAETISNELVSVLQKNS
PTFLESSFDIKSEVKKHAKSMLKELIKVGLPSFENLVAENVKPPKVDPATYGIIVPVLTS
LFNKVETAVGAKVSDEIWNYNSPDVSESEESLSDDFFD
>Pf LSA1(1-478) (full length synthetic gene) (SEQ ID NO: 12):
ggatccATGGGTACAAACAGTGAAAAAGATGAGATAATCAAAAGCAATTTACGATCTGGT
TCGTCTAACAGTCGTAACCGTATCAATGAAGAAAAACATGAAAAGAAACACGTATTATCG
CATAATAGCTATGAGAAAACCAAAAACAATGAGAATAATAAATTTTTTGATAAAGACAAG
GAGTTAACAATGTCCAATGTAAAGAACGTATCCCAAACGAATTTCAAATCACTTTTACGT
AACTTAGGTGTGTCCGAAAATATCTTCTTAAAAGAGAACAAATTGAATAAAGAGGGTAAA
CTAATTGAACACATTATTAATGATGATGACGACAAAAAGAAATATATCAAAGGCCAAGAC
GAGAATCGTCAAGAAGATCTTGAAGAAAAGGCGGCAGAACAACAAAGTGATCTTGAACAG
GAAAGACTTGCTAAAGAGAAATTGCAAGAACAACAGTCTGATTTAGAGCAAGAGCGTTTA
GCGAAAGAAAAATTACAAGAACGACTAGCAAAAGAAAAACTACAAGAGCAACAACGCGAT
TTGGAACAGGAACGTTTGGCAAAAGAGAAACTTCAAGAACAGCAACGCGATCTTGAACAA
CGAAAAGCAGATACCAAGAAGAATTTAGAACGCAAGAAAGAACACGGGGACGTTCTTGCC
GAAGATTTATATGGGCGATTAGAAATCCCAGCCATCGAATTACCATCTGAAAATGAACGA
GGCTATTATATCCCACATCAATCAAGCCTTCCTCAGGATAACAGAGGTAATAGCAGAGAT
TCTAAAGAAATTTCAATTATAGAGAAAACGAATAGAGAAAGTATCACTACAAACGTAGAA
GGACGCCGTGATATTCATAAAGGACATTTGGAAGAGAAGAAAGATGGGTCTATCAAACCG
GAACAGAAGGAAGATAAATCCGCTGACATTCAAAATCACACTCTTGAAACAGTTAACATT
AGCGACGTGAACGATTTTCAAATTTCTAAATATGAAGATGAAATTAGCGCTGAATATGAT
GATTCGCTTATTGACGAAGAAGAAGATGATGAAGACCTTGATGAATTTAAACCGATTGTT
CAATATGATAATTTTCAAGATGAAGAGAATATTGGAATCTATAAGGAATTAGAAGATTTA
ATCGAGAAAAATGAAAATTTAGATGATCTTGACGAAGGTATTGAAAAATCCTCTGAAGAA
CTTTCCGAAGAGAAAATTAAGAAAGGTAAAAAGTACGAGAAAACTAAAGACAACAATTTC
AAACCAAATGATAAAAGCCTTTATGACGAGCATATTAAAAAGTATAAAAACGATAAACAA
GTCAATAAAGAAAAAGAGAAGTTTATCAAATCTCTATTTCACATTTTTGACGGTGACAAT
GAAATCCTTCAAATTGTAGATGAATTGTCCGAAGATATCACAAAGTATTTTATGAAATTA
actagt
Corresponding amino acid sequence
(SEQ ID NO: 13)
MGTNSEKDEIIKSNLRSGSSNSRNRINEEKHEKKHVLSHNSYEKTKNNENNKFFDKDKEL
TMSNVKNVSQTNEKSLLRNLGVSENIFLKENKLNKEGKLIEHIINDDDDKKKYIKGQDEN
RQEDLEEKAAEQQSDLEQERLAKEKLQEQQSDLEQERLAKEKLQERLAKEKLQEQQRDLE
QERLAKEKLQEQQRDLEQRKADTKKNLERKKEHGDVLAEDLYGRLEIPAIELPSENERGY
YIPHQSSLPQDNRGNSRDSKEISIIEKTNRESITTNVEGRRDIHKGHLEEKKDGSIKPEQ
KEDKSADIQNHTLETVNISDVNDFQISKYEDEISAEYDDSLIDEEEDDEDLDEFKPIVQY
DNFQDEENIGIYKELEDLIEKNENLDDLDEGIEKSSEELSEEKIKKGKKYEKTKDNNFKP
NDKSLYDEHIKKYKNDKQVNKEKEKFIKSLFHIFDGDNEILQIVDELSEDITKYFMKL
>Pf TRAP synthetic gene (SEQ ID NO: 14):
ggatccAATGGTAGAGATGTACAGAACAATATCGTAGATGAGATCAAATACCGCGAAGAA
GTTTGCAATGATGAAGTTGATCTTTACTTGTTAATGGATTGTTCAGGTTCAAT CGTCGT
CATAACTGGGTCAATCACGCGGTTCCTTTGGCTATGAAACTTATTCAACAACTAAACCTA
AATGAATCTGCGATTCACTTGTATGTTAACATATTCTCGAACAATGCGAAAGAAATCATT
CGTTTACATTCGGATGCAAGCAAGAATAAAGAAAAAGCGTTGATAATCATACGAAGCTTA
CTAAGCACTAATCTTCCGTATGGCCGAACAAACTTATCTGATGCATTACTTCAGGTTAGA
AAACATTTGAATGATCGCATTAACCGTGAAAATGCAAATCAGTTGGTTGTGATTCTAACT
GATGGGATTCCTGATAGCATTCAAGATAGTCTTAAAGAATCACGAAAACTAAATGACCGT
GGTGTGAAAATCGCAGTTTTTGGAATTGGACAAGGCATCAATGTTGCTTTCAATCGATTC
TTAGTCGGGTGTCATCCATCCGACGGAAAGTGCAATTTGTATGCTGATTCTGCGTGGGAG
AATGTGAAAAACGTTATTGGACCATTCATGAAAGCCGTATGTGTTGAAGTAGAAAAGACA
GCTAGTTGCGGTGTGTGGGACGAATGGTCACCATGTAGTGTGACATGTGGCAAAGGCACA
CGCTCTCGCAAACGTGAAATACTTCACGAAGGATGCACCAGTGAATTACAAGAACAATGT
GAAGAAGAACGTTGTCCGCCAAAACGTGAACCACTAGATGTACCTGATGAACCAGAAGAT
GACCAACCGCGTCCGCGTGGTGACAACTTTGCTGTTGAGAAACCTGAAGAGAATATCATT
GACAATAACCCACAAGAGCCATCCCCAAACCCAGAGGAAGGTAAAGGGGAAAATCCAAAT
GGTTTCGACTTAGATGAAAATCCAGAAAATCCACCAAATCCGGATATTCCACAACAAGAA
CCAAACATTCCAGAAGATTCTGAAAAAGAAGTACCTAGTGATGTACCAAAGAATCCGGAG
GACGATAGAGAAGAAAACTTTGATATTCCTAAGAAACCGGAAAACAAACACGATAATCAA
AACAATCTTCCAAACGACAAATCAGATAGATCCATTCCTTATAGTCCTTTACCACCAAAA
GTACTTGATAATGAACGCAAACAATCGGACCCACAATCTCAAGACAACAATGGGAATCGT
CATGTGCCAAATAGCGAAGATAGAGAAACTAGACCTCATGGTCGTAACAATGAGAATCGA
TCATACAATCGCAAATACAATGATACGCCAAAACATCCAGAAAGAGAAGAACATGAAAAA
CCGGATAACAATAAGAAAAAGGGAGGTAGTGACAACAAGTATAAGATTGCAGGTGGCATT
GCAGGCGGATTAGCATTACTTGCTTGCGCAGGCTTAGCCTACAAATTCGTAGTCCCGGGT
GCAGCTACGCCTTATGCCGGAGAGCCAGCTCCGTTTGATGAAACATTAGGAGAAGAAGAT
AAGGATTTAGATGAGCCTGAGCAATTCAGATTACCTGAAGAAAATGAATGGAATcaattg
Corresponding amino acid sequence
(SEQ ID NO: 15)
NGRDVQNNIVDEIKYREEVCNDEVDLYLLMDCSGSIRRHNWVNHAVPLAMKLIQQLNLNE
SAIHLYVNIFSNNAKEIIRLHSDASKNKEKALIIIRSLLSTNLPYGRTNLSDALLQVRKH
LNDRINRENANQLVVILTDGIPDSIQDSLKESRKLNDRGVKIAVFGIGQGINVAENRELV
GCHPSDGKCNLYADSAWENVKNVIGPFMKAVCVEVEKTASCGVWDEWSPCSVTCGKGTRS
RKREILHEGCTSELQEQCEEERCPPKREPLDVPDEPEDDQPRPRGDNFAVEKPEENIIDN
NPQEPSPNPEEGKGENPNGFDLDENPENPPNPDIPQQEPNIPEDSEKEVPSDVPKNPEDD
REENFDIPKKPENKHDNQNNLPNDKSDRSIPYSPLPPKVLDNERKQSDPQSQDNNGNRHV
PNSEDRETRPHGRNNENRSYNRKYNDTPKHPEREEHEKPDNNKKKGGSDNKYKIAGGIAG
GLALLACAGLAYKFVVPGAATPYAGEPAPFDETLGEEDKDLDEPEQFRLPEENEWN
>Pf TRAP(24-497)
(SEQ ID NO: 16)
ggatccAATGGTAGAGATGTACAGAACAATATCGTAGATGAGATCAAATACCGCGAAGAA
GTTTGCAATGATGAAGTTGATCTTTACTTGTTAATGGATTGTTCAGGTTCAATTCGTCGT
CATAACTGGGTCAATCACGCGGTTCCTTTGGCTATGAAACTTATTCAACAACTAAACCTA
AATGAATCTGCGATTCACTTGTATGTTAACATATTCTCGAACAATGCGAAAGAAATCATT
CGTTTACATTCGGATGCAAGCAAGAATAAAGAAAAAGCGTTGATAATCATACGAAGCTTA
CTAAGCACTAATCTTCCGTATGGCCGAACAAACTTATCTGATGCATTACTTCAGGTTAGA
AAACATTTGAATGATCGCATTAACCGTGAAAATGCAAATCAGTTGGTTGTGATTCTAACT
GATGGGATTCCTGATAGCATTCAAGATAGTCTTAAAGAATCACGAAAACTAAATGACCGT
GGTGTGAAAATCGCAGTTTTTGGAATTGGACAAGGCATCAATGTTGCTTTCAATCGATTC
TTAGTCGGGTGTCATCCATCCGACGGAAAGTGCAATTTGTATGCTGATTCTGCGTGGGAG
AATGTGAAAAACGTTATTGGACCATTCATGAAAGCCGTATGTGTTGAAGTAGAAAAGACA
GCTAGTTGCGGTGTGTGGGACGAATGGTCACCATGTAGTGTGACATGTGGCAAAGGCACA
CGCTCTCGCAAACGTGAAATACTTCACGAAGGATGCACCAGTGAATTACAAGAACAATGT
GAAGAAGAACGTTGTCCGCCAAAACGTGAACCACTAGATGTACCTGATGAACCAGAAGAT
GACCAACCGCGTCCGCGTGGTGACAACTTTGCTGTTGAGAAACCTGAAGAGAATATCATT
GACAATAACCCACAAGAGCCATCCCCAAACCCAGAGGAAGGTAAAGGGGAAAATCCAAAT
GGTTTCGACTTAGATGAAAATCCAGAAAATCCACCAAATCCGGATATTCCACAACAAGAA
CCAAACATTCCAGAAGATTCTGAAAAAGAAGTACCTAGTGATGTACCAAAGAATCCGGAG
GACGATAGAGAAGAAAACTTTGATATTCCTAAGAAACCGGAAAACAAACACGATAATCAA
AACAATCTTCCAAACGACAAATCAGATAGATCCATTCCTTATAGTCCTTTACCACCAAAA
GTACTTGATAATGAACGCAAACAATCGGACCCACAATCTCAAGACAACAATGGGAATCGT
CATGTGCCAAATAGCGAAGATAGAGAAACTAGACCTCATGGTCGTAACAATGAGAATCGA
TCATACAATCGCAAATACAATGATACGCCAAAACATCCAGAAAGAGAAGAACATGAAAAA
CCGGATAACAATAAGAAAAAGGGAGGTAGTGACAACAAGTATAAGATTcaattg
Corresponding amino acid sequence
(SEQ ID NO: 17)
NGRDVQNNIVDEIKYREEVCNDEVDLYLLMDCSGSIRRHNWVNHAVPLAMKLIQQLNLNE
SAIHLYVNIFSNNAKEIIRLHSDASKNKEKALIIIRSLLSTNLPYGRTNLSDALLQVRKH
LNDRINRENANQLVVILTDGIPDSIQDSLKESRKLNDRGVKIAVFGIGQGINVAFNRFLV
GCHPSDGKCNLYADSAWENVKNVIGPFMKAVCVEVEKTASCGVWDEWSPCSVTCGKGTRS
RKREILHEGCTSELQEQCEEERCPPKREPLDVPDEPEDDQPRPRGDNFAVEKPEENIIDN
NPQEPSPNPEEGKGENPNGFDLDENPENPPNPDIPQQEPNIPEDSEKEVPSDVPKNPEDD
REENFDIPKKPENKHDNQNNLPNDKSDRSIPYSPLPPKVLDNERKQSDPQSQDNNGNRHV
PNSEDRETRPHGRNNENRSYNRKYNDTPKHPEREEHEKPDNNKKKGGSDNKYKI

Construct IDs:

| Lm11 | Lm677 | Construct |
|---|---|---|
| BH2200 | BH2214 | Pf-LSA1 FL (residues 1-478; SEQ ID NO: 13) |
| BH2228 | BH2230 | Pf-LSA1(1-277) |
| BH2212 | BH2226 | Pf-LSA1(236-478) |
| BH2202 | BH2216 | Pf-CelTOS 1-158 (residues 1-158; SEQ ID NO: 11) |
| BH2232 | BH2233 | Pf-CelTOS(1-110) |
| BH2245 | BH2246 | Pf-CelTOS(1-110 + 122-158) |
| BH2204 | BH2218 | Pf-CSP FL (residues 1-235; SEQ ID NO: 7) |
| BH2210 | BH2224 | Pf-CSP(1-224) (SEQ ID NO: 9) |
| BH2500 | BH2510 | Pf-TRAP FL (residues 24-559; SEQ ID NO: 15) |
| BH2526 | BH2538 | Pf-TRAP 24-497 (SEQ ID NO: 17) |
| BH2528 | BH2540 | Pf-TRAP 24-291 |
| BH2530 | BH2542 | Pf-TRAP278-559 |

| Strain | Construct at tRNA^{Arg} | Construct at comK |
|---|---|---|
| Lm11 | none | none |
| BH137 | Postive control (OVA) | none |
| BH2200 | ActAN100-LSAl-SL8 | none |
| BH2358 | none | ActAN100-LSA1-SL8 |
| BH2202 | ActAN100-CelTOS-SL8 | none |
| BH2360 | none | ActAN100-CelTOS-SL8 |
| BH2210 | ActAN100-CSP-SL8 | none |
| BH2362 | none | ActAN100-CSP-SL8 |
| BH2364 | ActAN100-CelTOS-SL8 | ActAN100-LSA1-SL8 |
| BH2366 | ActAN100-CelTOS-SL8 | ActAN100-CelTOS-SL8 |
| BH2368 | ActAN100-CelTOS-SL8 | ActAN100-CSP-SL8 |
| BH2370 | ActAN100-LSAl-SL8 | ActAN100-CSP-SL8 |

### Example 2

### In Vitro Cell Culture

J774, P815, and EL-4 cells were cultured in T cell media (RPMI media (Invitrogen, Carlsbad, Calif.) supplemented with 10% FBS (Hyclone, Logan, Utah), 5e4 I.U..5e4 μg penicillin/streptomycin (Mediatech, Manassas, Va.), 1 × non-essential amino acids (Mediatech, Manassas, Va.), 2 mM L-glutamine (Mediatech, Manassas, Va.), HEPES buffer (Invitrogen, Carlsbad, Calif.), 1 mM sodium pyruvate (Sigma, St. Louis, Mo.), and 50 μM β-mercaptoethanol (Sigma, St. Louis, Mo.)). DC2.4 and B3Z hybridoma were cultured in T cell media without penicillin/streptomycin.

### Example 3

### Preparation of Peptides

Peptides for OVA₂₅₇₋₂₆₄ (SIINFEKL, SL8), p60₂₁₇₋₂₂₅ (KYGVSQDI), LLO₉₁₋₉₉ (GYKDGNEYI), and LLO₁₉₀₋₂₀₁ (NEKYAQAYPNVS) were synthesized by Invitrogen (Carlsbad, Calif.). Peptides for LSA-1₁₆₇₁₋₁₆₇₉ (YYIPHQSSL), Pf CSP₃₉₋₄₇ (NYDNAGTNL), Pb CSP₂₅₂₋₂₆₀ (SYIPSAEKI), and HPV16 E7₄₉₋₅₇ (RAHYNIVTF) were synthesized by Synthetic Biomolecules (San Diego, Calif.). Ce1TOS peptide library consisting of 15-mer peptides that overlap by 11 amino acids and span the sequence of Ce1TOS was synthesized by JPT Peptide Technology (Berlin, Germany). Ce1TOS peptide library includes peptides #25 (VAENVKPPKVDPATY), #26 (VKPPKVDPATYGIIV), #34 (VSDEIWNYNSPDVSE), and #35 (IWNYNSPDVSESEES).

### Example 4

### Immunizations

6-12 week old female C57BL/6 and Balb/c mice were obtained from Charles River Laboratories (Wilmington, Mass.). Studies were performed under animal protocols approved by the Aduro (and Anza) Institutional Animal Care and Use Committee. Live-attenuated bacteria were prepared for immunization from overnight cultures grown in yeast extract media. Bacteria were diluted in Hank's balanced salt solution (HBSS) for injection. Live-attentuated bacteria were administered i.v. into tail vein in 200 μL volume. Injection stocks of live-attenuated bacteria were plated to confirm colony forming units (CFU).

### Example 5

### Assessment of Antigen Expression and Immune Response

a. Western Blots

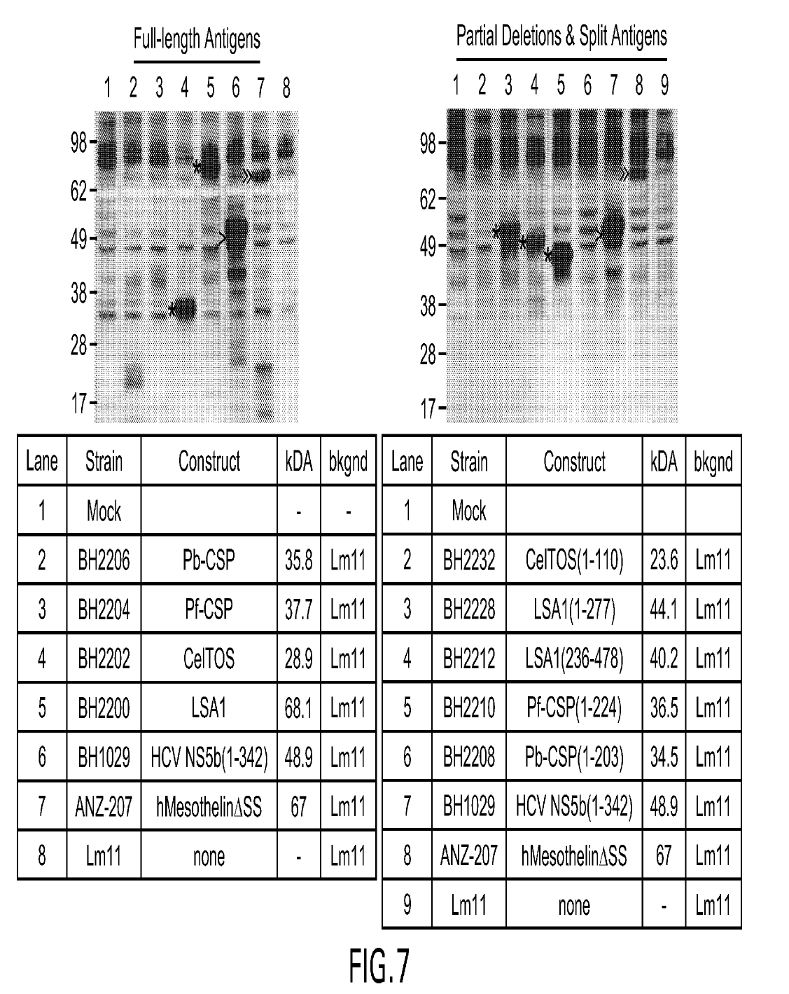

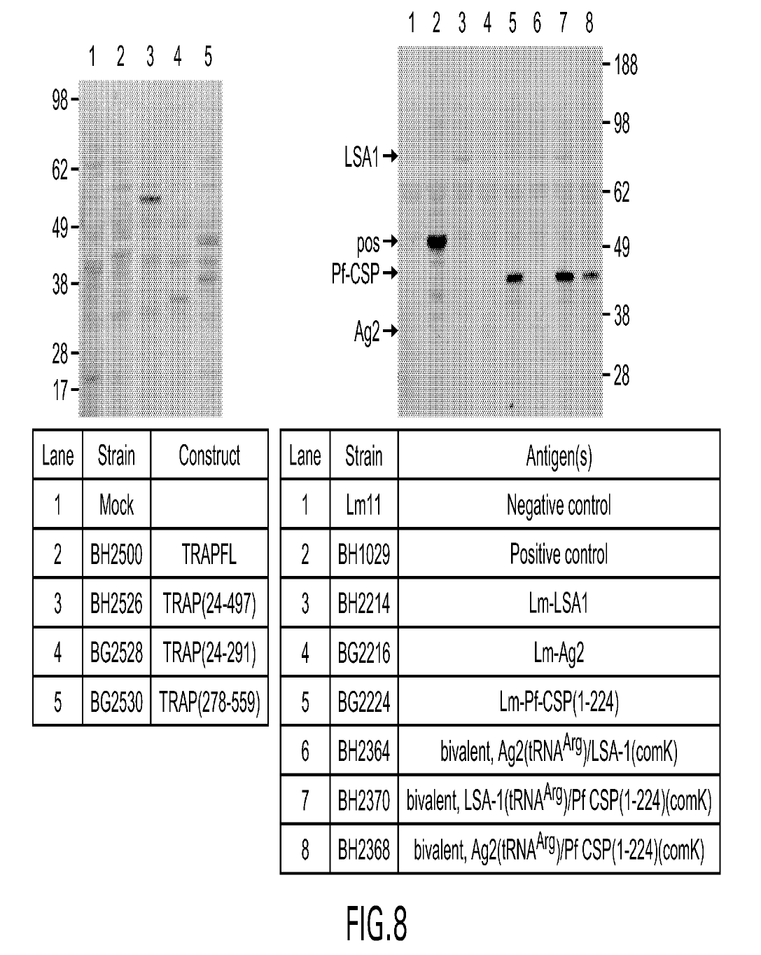

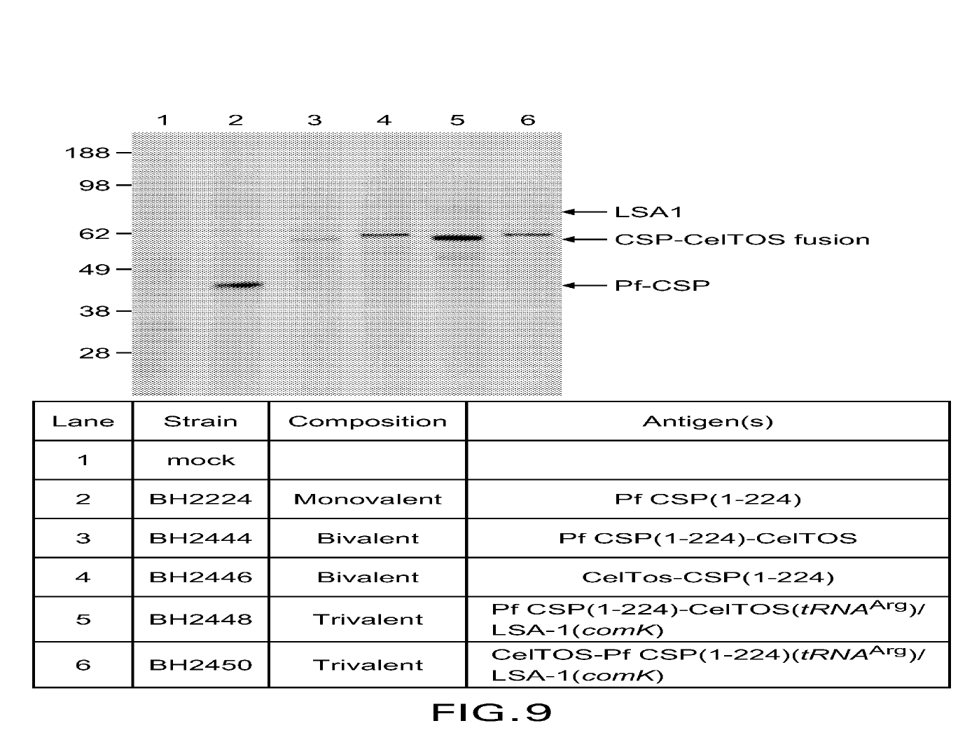

Western blots from broth culture were performed on equivalent amounts of TCA-precipitated supernatants of bacterial cultures grown in yeast extract media to an OD₆₀₀ of 0.7 (late log). For western blots from Lm infected DC2.4 cells were inoculated with a multiplicity of infection (MOI) of 10 for 1 hour, the cells were washed 3× with PBS and DMEM media supplemented with 50 μg/mL gentamycin. Cells were harvested at 7 hours post infection. Cells were lysed with SDS sample buffer, collected and run on 4-12% polyacrylamide gels and transferred to nitrocellulose membranes for western blot analysis. All western blots utilized a polyclonal antibody raised against the mature N-terminus of the ActA protein and were normalized to p60 expression (an unrelated Lm protein) with an anti-p60 monoclonal antibody. Antigen detection was visualized either by enhanced chemiluminescence (ECL) or visualized and quantitated with the Licor Odyssey IR detection system. Results for the Pf antigen constructs are depicted in FIGS. 7-9.

b. B3Z Assay

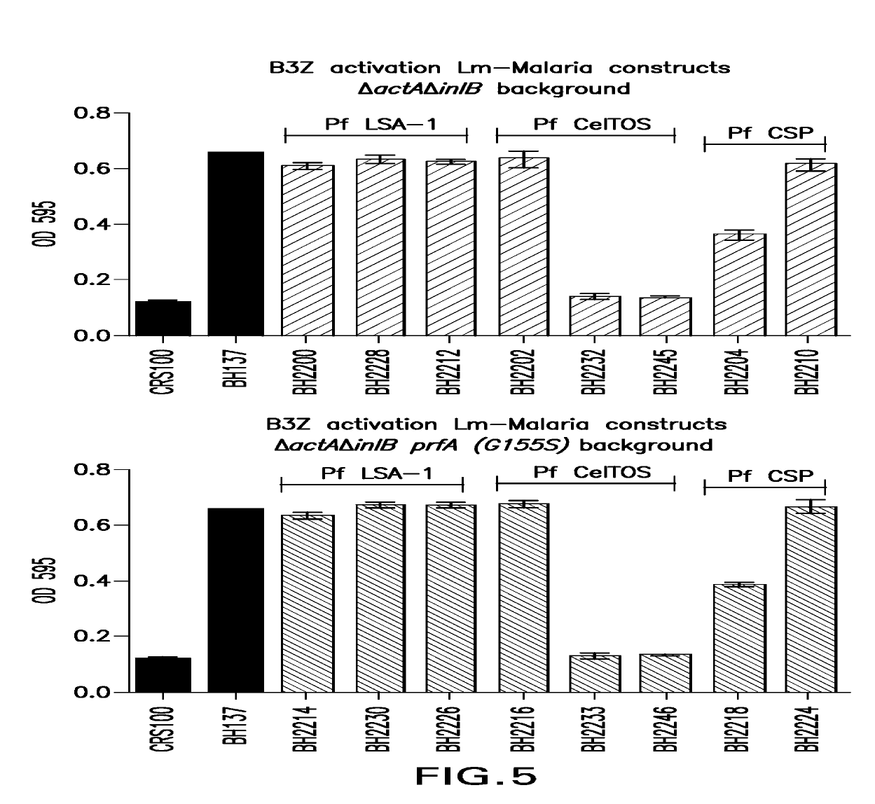

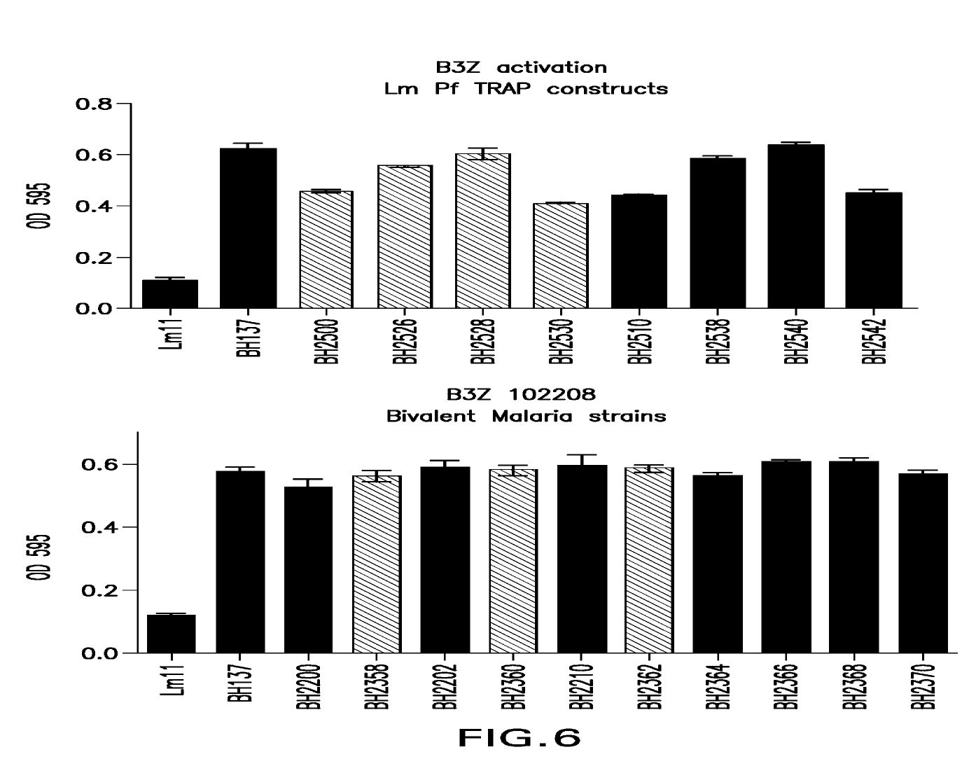

DC2.4 cells were infected with various malaria vaccine strains, and then incubated with the OVA₂₅₇₋₂₆₄-specific T cell hybridoma, B3Z. Presentation of SIINFEKL epitope on H-2 K^{b} class I molecules was assessed by measuring β-galactosidase expression using a chromogenic substrate. Results for the Pf antigen constructs are depicted in FIGS. 5 and 6.

c. Reagents for Flow Cytometry

CD4 FITC or Alexa 700 (L3T4, clone GK1.5), CD8 APC-Alexa 750 (Ly-2, clone 53-6.7), TNF PE or PE-Cy7 (clone MP6-XT22), IFN-γ APC (clone XMG1.2), IL-2-PE (clone JES6-5H4), and CCR7-biotin (clone 4B 12) were purchased from eBioscience (San Diego, Calif.). CD8a PerCP (clone 53-6.7) was purchased from BD Biosciences (San Jose, Calif.). PE-Texas red streptavidin conjugate and GrVid were purchased from Invitrogen (San Diego, Calif.).

d. Intracellular Staining of Antigen-Specific T Cells

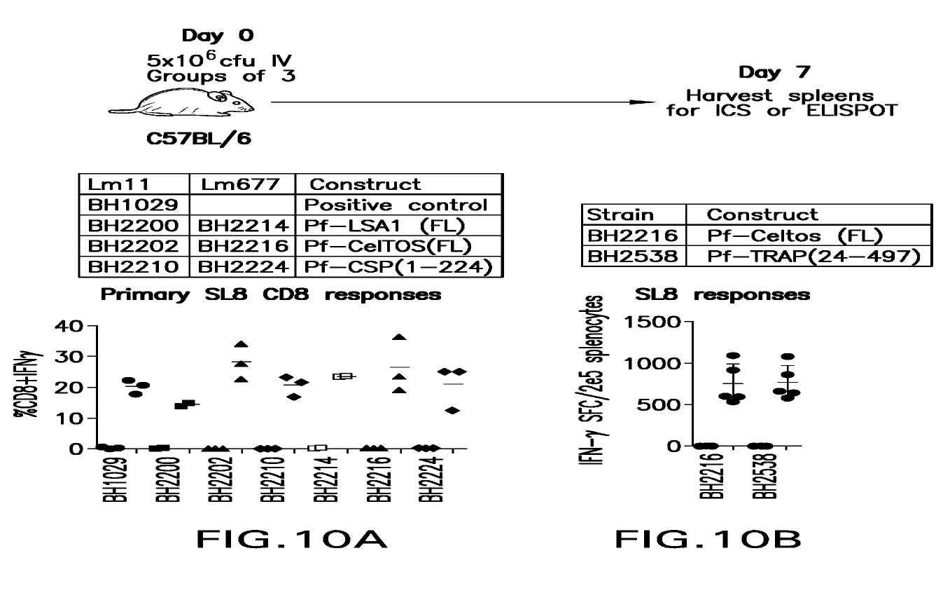

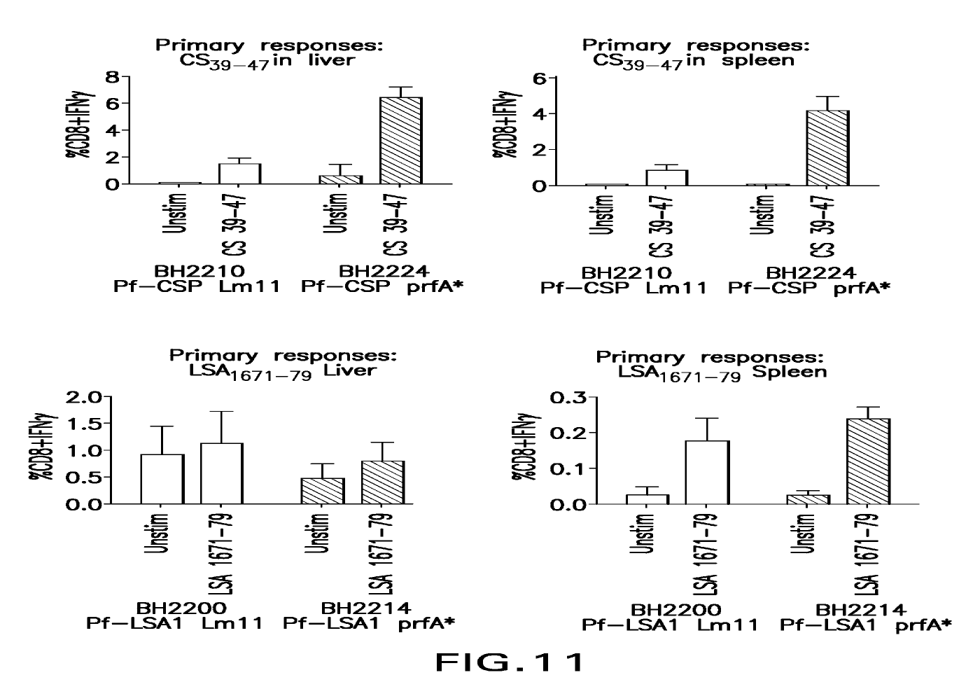

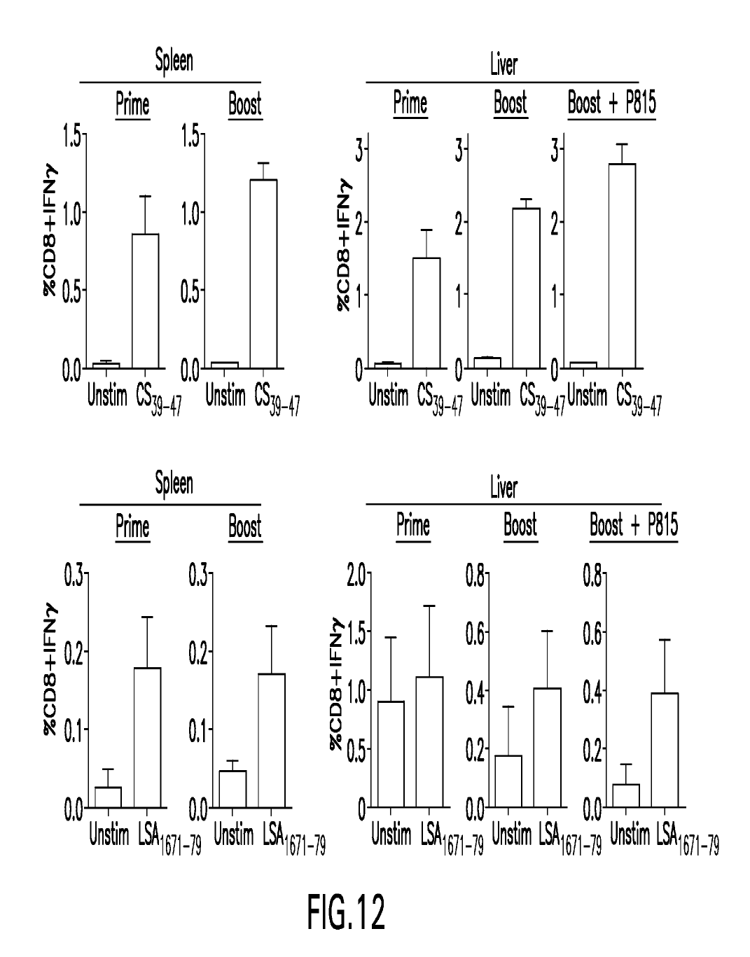

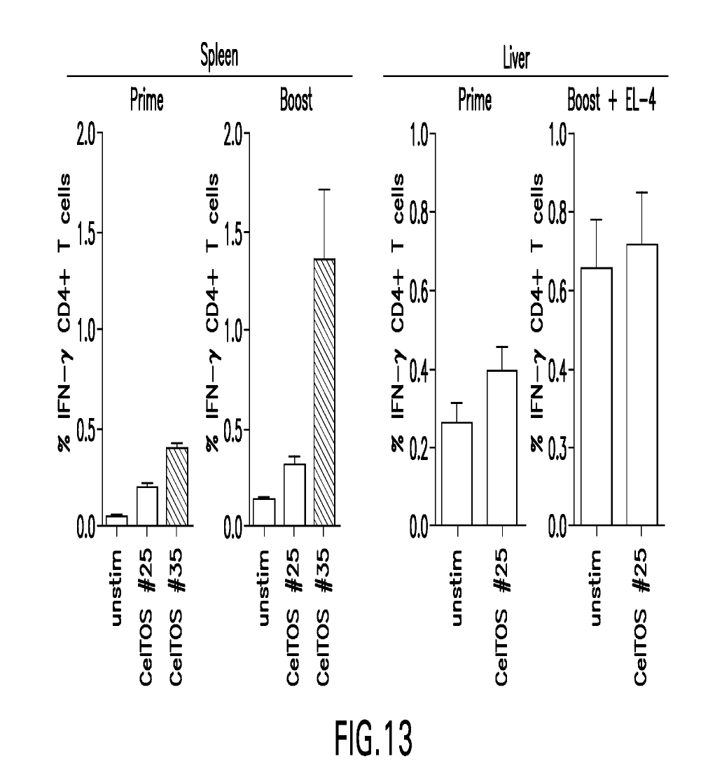

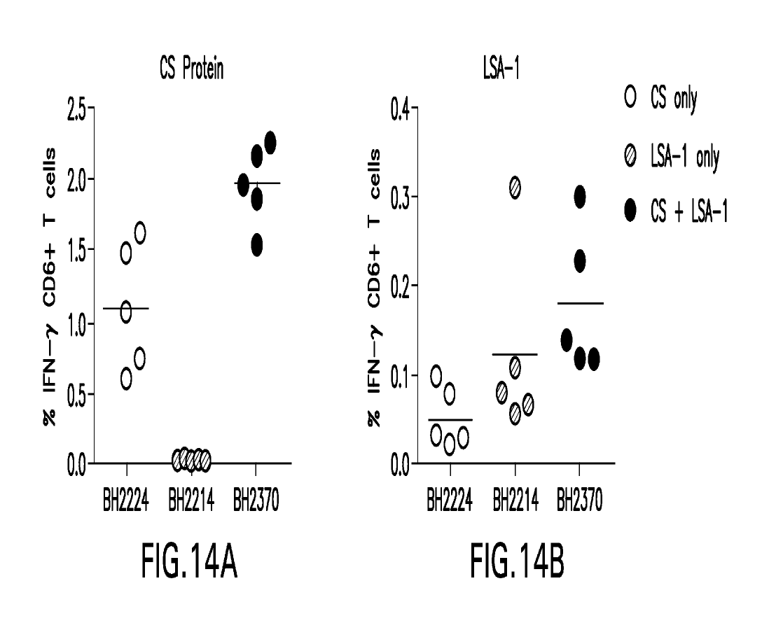

Splenocytes and lymphocytes, isolated from liver or peripheral blood using Percoll (Sigma, St. Louis, Mo.) or Lympholyte-Mammal (Cedarlane Labs, Burlington, N.C.) respectively, were incubated with the appropriate peptides at 1 μM for five hours in presence of brefeldin A (BD Biosciences, San Jose, Calif.). Equal numbers of P815 or EL-4 cells were incubated with lymphocytes from liver and blood. Stimulated cells were surface stained for CD4 and CD8, then fixed and permeabilized using the cytofix/cytoperm kit (BD Biosciences, San Jose, Calif.). Cells were then stained for IFN-γ, TNF-α and/or IL-2. Samples were acquired using a FACSCanto flow cytometer (BD Biosciences). Data were gated to include exclusively CD4+ or CD8+ events, then the percentage of these cells expressing IFN-γ, TNF-α, or IL-2 determined. Data was analyzed using FlowJo software (Treestar, Ashland, Oreg.). Results are depicted in FIGS. 10-15.

e. ELISPOT Assay

ELISPOT assays were performed using a murine IFN-γ ELISPOT Spot pair (BD Biosciences, San Diego, Calif.) and PVDF membrane 96-well plate (Millipore, Billerica, Mass.). 2×10⁵ splenocytes or 1×10⁵ lymphocytes from liver or blood were incubated in each well with the appropriate peptide overnight at 37° C. and developed using alkaline phosphatase detection reagents (Invitrogen, San Diego, Calif.). An equal number of antigen presenting cells, either P815 or EL-4 cells, were included with blood and liver lymphocytes. Plates were scanned and quantified using Immunospot plate reader and software (CTL Ltd, Cleveland, Ohio).

### Example 6

### Results

As can be seen from the data presented herein, monovalent (meaning expressing a single Plasmodium antigen sequence) Listeria based vaccine strains encoding pre-erythrocytic P. falciparum antigens CSP, Ce1TOS, LSA1, or TRAP express and secrete malaria antigens within infected antigen presenting cells. Malaria antigens expressed and secreted from Listeria monocytogenes within an infected APC are processed and presented in context of MHC class I molecules (B3Z data). Monovalent Listeria based vaccine strains encoding pre-erythrocytic P. falciparum antigens CSP, Ce1TOS, LSA1, or TRAP also induce malaria-antigen specific immunity in mice that can be detected in spleen, blood and liver.

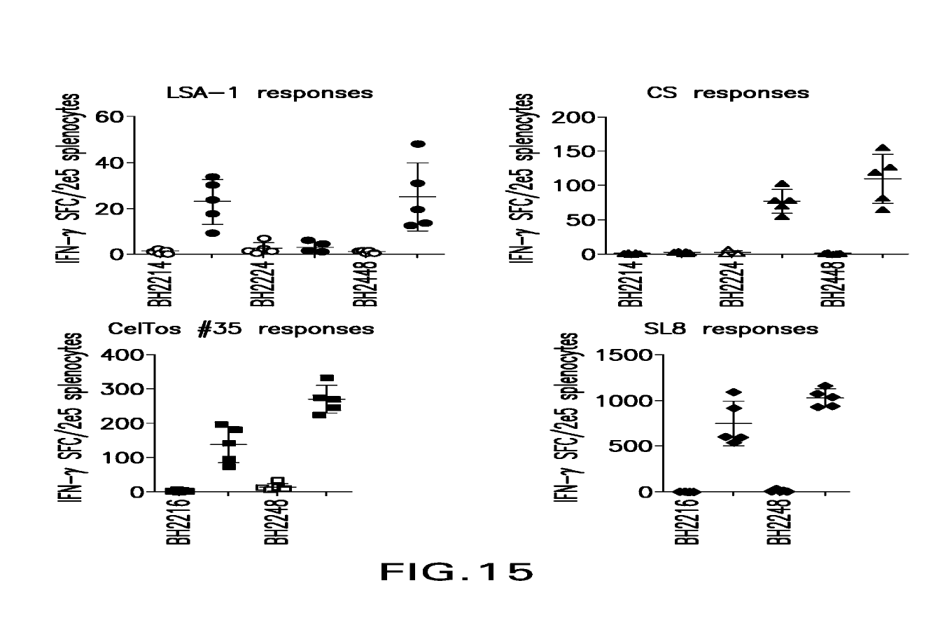

Multiple (two or three) malaria antigens can be expressed and secreted within infected APCs from the same Listeria strain (refereed to herein as bi- and trivalent strains). Expression is comparable to the respective monovalent strains. Bivalent Listeria vaccine strains with antigens either expressed from two Listeria loci or as fusion proteins from one locus induce potent multi-antigen T-cell responses. The magnitude of the immune response is comparable to the respective monovalent strains (FIG. 15.)

Trivalent Listeria vaccine strains induce potent antigen specific T cell responses to each of Ce1TOS, LSA1, and CSP and make a promising prophylactic vaccine for the prevention of malaria.

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims.

We claim:

1. A method of inducing a T-cell response to a Plasmodium antigen in a subject, said method comprising:
administering to said subject a composition comprising a bacterium which expresses one or more immunogenic Plasmodium-derived antigen polypeptides, the amino acid sequence of which comprise
a polypeptide sequence derived from wild-type Plasmodium LSA1, Ce1TOS, CSP, and/or TRAP sequences, wherein said amino acid sequences are derived by (i) codon optimization of the wild-type sequence for expression in said bacterium, (ii) deletion of at least one hydrophobic region present in the wild-type sequence, and/or (iii) in the case of LSA1 and CSP, minimization of repeat units present in the wild-type sequence
under conditions selected to induce said T cell response in said subject.

2. The method of claim 1 wherein said immunogenic Plasmodium-derived antigen polypeptide(s) comprise one or more amino acid sequences selected from the group consisting of SEQ ID NOS: 7, 9, 11, 13, 15, and 17; or modifications or fragments thereof sharing at least 90% identity with at least 30 amino acids from these sequences.

3. The method of claim 1 wherein said immunogenic Plasmodium-derived antigen polypeptide(s) comprise amino acid sequences derived from at least two of the wild-type Plasmodium LSA1, Ce1TOS, CSP, and TRAP sequences.

4. The method of claim 1 wherein said immunogenic Plasmodium-derived antigen polypeptide(s) comprise amino acid sequences derived from at least three of the wild-type Plasmodium LSA1, Ce1TOS, CSP, and TRAP sequences.

5. The method of claim 1 wherein said immunogenic Plasmodium-derived antigen polypeptide(s) comprise amino acid sequences derived from one or more of Plasmodium falciparum LSA1, Ce1TOS, CSP, and TRAP sequences.

6. The method of claim 1, wherein the bacterium is Listeria monocytogenes comprising a nucleic acid sequence encoding said one or more immunogenic Plasmodium-derived antigen polypeptides integrated into the genome of said bacterium.

7. The method of claim 6, wherein the bacterium is an actA deletion mutant or an actA insertion mutant, an inlB deletion mutant or an inlB insertion mutant or a ΔactA/ΔinlB mutant comprising both an actA deletion or an actA insertion and an inlB deletion or an inlB insertion.

8. The method of claim 6, wherein a polynucleotide encoding one or more of said immunogenic Plasmodium-derived antigen polypeptide(s) has been integrated into a virulence gene of said bacterium, and the integration of the polynucleotide disrupts expression of the virulence gene or disrupts a coding sequence of the virulence gene.

9. The method of claim 8, wherein the virulence gene is actA or inlB.

10. The method of claim 6, wherein the bacterium is an attenuated Listeria monocytogenes.

11. The method of claim 10, wherein the bacterium is Lm ΔactA/ΔinlB.

12. The method of claim 8, wherein the bacterium further comprises a genetic mutation that attentuates the ability of the bacterium to repair nucleic acid.

13. The method of claim 12, wherein the genetic mutation is in one or more genes selected from phrB, uvrA, uvrB, uvrC, uvrD and recA.

14. The method of claim 10, wherein the bacterium is a Listeria monocytogenes prfA mutant, the genome of which encodes a prfA protein which is constitutively active.

15. The method of claim 6, wherein the bacterium is a killed but metabolically active Listeria monocytogenes.

16. The method of claim 15, wherein the bacterium is a Listeria monocytogenes prfA mutant, the genome of which encodes a prfA protein which is constitutively active.

17. The method of claim 6, wherein the nucleic acid sequence is codon optimized for expression by Listeria monocytogenes.

18. The method of claim 6, wherein said conditions selected to induce said T cell response in said subject comprise administering said Listeria monocytogenes by one or more routes of administration selected from the group consisting of orally, intramuscularly, intravenously, intradermally, and subcutaneously to said subject.

19. The method of claim 1, wherein said immunogenic Plasmodium-derived antigen polypeptide(s) are expressed as a fusion protein comprising a secretory signal sequence.

20. The method of claim 19, wherein the secretory signal sequence is a Listeria monocytogenes ActA signal sequence.

21. The method of claim 20, wherein said immunogenic Plasmodium-derived antigen polypeptide(s) are expressed as a fusion protein comprising an in frame ActA-N100 sequence selected from the group consisting of SEQ ID NO: 37, SEQ ID NO: 38, and SEQ ID NO: 39, or an amino acid sequence having at least 90% sequence identity to said ActA-N100 sequence.

22. The method of claim 1, wherein said method comprises administering a Listeria monocytogenes expressing a fusion protein comprising:
an ActA-N100 sequence selected from the group consisting of SEQ ID NO: 37, SEQ ID NO: 38, SEQ ID NO: 39, SEQ ID NO: 40 or an amino acid sequence having at least 90% sequence identity to said ActA-N100 sequence; and one or more of:
a Plasmodium-derived amino acid comprising the sequence of SEQ ID NO: 7, or a modification or fragment thereof sharing at least 90% identity with at least 30 amino acids thereof,
a Plasmodium-derived amino acid comprising the sequence of SEQ ID NO: 9, or a modification or fragment thereof sharing at least 90% identity with at least 30 amino acids thereof,
a Plasmodium-derived amino acid comprising the sequence of SEQ ID NO: 11, or a modification or fragment thereof sharing at least 90% identity with at least 30 amino acids thereof,
a Plasmodium-derived amino acid comprising the sequence of SEQ ID NO: 13, or a modification or fragment thereof sharing at least 90% identity with at least 30 amino acids thereof,
a Plasmodium-derived amino acid comprising the sequence of SEQ ID NO: 15, or a modification or fragment thereof sharing at least 90% identity with at least 30 amino acids thereof, and
a Plasmodium-derived amino acid comprising the sequence of SEQ ID NO: 17, or a modification or fragment thereof sharing at least 90% identity with at least 30 amino acids thereof,
wherein said fusion protein is expressed from a nucleic acid sequence operably linked to a Listeria monocytogenes ActA promoter.

23. The method of claim 1, wherein said subject has a malaria infection.

24. The method of claim 1, wherein said subject does not have a malaria infection and is being treated prophylactically.

25. The method of claim 1, wherein said composition, when delivered to said subject, induces an increase in the serum concentration of one or more proteins selected from the group consisting of IL-12p70, IFN-γ, IL-6, TNF α, and MCP-1 at 24 hours following said delivery; and induces a CD4+ and/or CD8+ antigen-specific T cell response against one or more of said immunogenic Plasmodium-derived antigen polypeptide(s).

26. The method of claim 1, wherein deletion of at least one hydrophobic region present in the wild-type sequence comprises deletion of the signal sequence present in the wild-type sequence.

27. A composition comprising:
a bacterium which expresses one or more immunogenic Plasmodium-derived antigen polypeptides, the amino acid sequence of which comprise
a polypeptide sequence derived from wild-type Plasmodium LSA1, Ce1TOS, CSP, and/or TRAP sequences, wherein said amino acid sequences are derived by (i) codon optimization of the wild-type sequence for expression in said bacterium, (ii) deletion of at least one hydrophobic region present in the wild-type sequence, and/or (iii) in the case of LSA1 and CSP, minimization of repeat units present in the wild-type sequence.

28. The composition of claim 27 wherein said immunogenic Plasmodium-derived antigen polypeptide(s) comprise one or more amino acid sequences selected from the group consisting of SEQ ID NOS: 7, 9, 11, 13, 15, and 17; or modifications or fragments thereof sharing at least 90% identity with at least 30 amino acids from these sequences.

29. The composition of claim 27 wherein said immunogenic Plasmodium-derived antigen polypeptide(s) comprise amino acid sequences derived from at least two of the wild-type Plasmodium LSA1, Ce1TOS, CSP, and TRAP sequences.

30. The composition of claim 27 wherein said immunogenic Plasmodium-derived antigen polypeptide(s) comprise amino acid sequences derived from at least three of the wild-type Plasmodium LSA1, Ce1TOS, CSP, and TRAP sequences.

31. The composition of claim 27, wherein the bacterium is Listeria monocytogenes comprising said nucleic acid sequence integrated into the genome of said bacterium.

32. The composition of claim 31, wherein the bacterium is an actA deletion mutant or an actA insertion mutant, an inlB deletion mutant or an inlB insertion mutant or a ΔactA/ΔinlB mutant comprising both an actA deletion or an actA insertion and an inlB deletion or an inlB insertion.

33. The composition of claim 31, wherein a polynucleotide encoding one or more of said immunogenic Plasmodium-derived antigen polypeptide(s) has been integrated into a virulence gene of said bacterium, and the integration of the polynucleotide disrupts expression of the virulence gene or disrupts a coding sequence of the virulence gene.

34. The composition of claim 33, wherein the virulence gene is actA or inlB.

35. The composition of claim 31 wherein the bacterium is an attenuated Listeria monocytogenes.

36. The composition of claim 35, wherein the bacterium is Lm ΔactA/ΔinlB.

37. The composition of claim 33, wherein the bacterium further comprises a genetic mutation that attentuates the ability of the bacterium to repair nucleic acid.

38. The composition of claim 37, wherein the genetic mutation is in one or more genes selected from phrB, uvrA, uvrB, uvrC, uvrD and recA.

39. The composition of claim 35, wherein the bacterium is a Listeria monocytogenes prfA mutant, the genome of which encodes a prfA protein which is constitutively active.

40. The composition of claim 36, wherein the bacterium is a killed but metabolically active Listeria monocytogenes.

41. The composition of claim 31, wherein the bacterium is a Listeria monocytogenes prfA mutant, the genome of which encodes a prfA protein which is constitutively active.

42. The composition of claim 31, wherein the nucleic acid sequence is codon optimized for expression by Listeria monocytogenes.

43. The composition of claim 27, wherein said composition further comprises a pharmaceutically acceptable excipient.

44. The composition of claim 27, wherein said nucleic acid molecule encodes said immunogenic said immunogenic Plasmodium-derived antigen polypeptide(s) as a fusion protein comprising a secretory signal sequence.

45. The composition of claim 44, wherein the secretory signal sequence is a Listeria monocytogenes ActA signal sequence.

46. The composition of claim 45, wherein said nucleic acid molecule encodes said immunogenic Plasmodium-derived antigen polypeptide(s) as a fusion protein comprising an in frame ActA-N100 sequence selected from the group consisting of SEQ ID NO: 37, SEQ ID NO: 38, and SEQ ID NO: 39, or an amino acid sequence having at least 90% sequence identity to said ActA-N 100 sequence.

47. The composition of claim 27, wherein said composition comprises a Listeria monocytogenes which comprises a nucleic acid molecule, the sequence of which encodes a fusion protein comprising:
an ActA-N100 sequence selected from the group consisting of SEQ ID NO: 37, SEQ ID NO: 38, SEQ ID NO: 39, SEQ ID NO: 40 or an amino acid sequence having at least 90% sequence identity to said ActA-N100 sequence; and one or more of:
a Plasmodium-derived amino acid comprising the sequence of SEQ ID NO: 7, or a modification or fragment thereof sharing at least 90% identity with at least 30 amino acids thereof,
a Plasmodium-derived amino acid comprising the sequence of SEQ ID NO: 9, or a modification or fragment thereof sharing at least 90% identity with at least 30 amino acids thereof,
a Plasmodium-derived amino acid comprising the sequence of SEQ ID NO: 11, or a modification or fragment thereof sharing at least 90% identity with at least 30 amino acids thereof,
a Plasmodium-derived amino acid comprising the sequence of SEQ ID NO: 13, or a modification or fragment thereof sharing at least 90% identity with at least 30 amino acids thereof,
a Plasmodium-derived amino acid comprising the sequence of SEQ ID NO: 15, or a modification or fragment thereof sharing at least 90% identity with at least 30 amino acids thereof, and
a Plasmodium-derived amino acid comprising the sequence of SEQ ID NO: 17, or a modification or fragment thereof sharing at least 90% identity with at least 30 amino acids thereof,
wherein said nucleic acid molecule encoding said fusion protein is operably linked to a Listeria monocytogenes ActA promoter.

48. The composition of claim 31, wherein said immunogenic Plasmodium-derived antigen polypeptide(s) comprise one or more contiguous Plasmodium-derived amino acid sequences having no region of hydrophobicity that exceeds the peak hydrophobicity of Listeria ActA-N100.

49. The composition of claim 27, wherein deletion of at least one hydrophobic region present in the wild-type sequence comprises deletion of the signal sequence present in the wild-type sequence.

50-53. (canceled)
